(19) United States
(12) Patent Application Publication
(10) Pub. No.: US 2010/0197591 A1
(43) Pub. Date: Aug. 5, 2010

(54) 4-AMINO-7,8-DIHYDROPYRIDO[4,3-d]PYRIMIDIN-5(6H)-ONE DERIVATIVES

(75) Inventors: Gary E. Aspnes, Rockville, RI (US); Robert L. Dow, Groton, CT (US); Michael J. Munchhof, Salem, CT (US)

Correspondence Address: PFIZER INC.;PATENT DEPARTMENT, Bld 114 M/S 9114, EASTERN POINT ROAD, GROTON, CT, 06340, US

(73) Assignee: pfizer Inc

(21) Appl. No.: 12/697,715

(22) Filed: Feb. 1, 2010

Related U.S. Application Data
(60) Provisional application No. 61/149,863, filed on Feb. 4, 2009.
(60) Provisional application No. 61/150,028, filed on Feb. 5, 2009.
(60) Provisional application No. 61/285,408, filed on Dec. 10, 2009.

(51) Int. Cl. A61K 31/519; C07D 471/04; A61P 31/04; A61P 31/10; A61K 31/536; A61K 31/55; A61K 38/16; A61K 31/4985; A61P 3/10

(52) U.S. Cl. 514/12; 544/279; 514/264.11; 514/230.5; 514/217.01; 514/250; 514/249

(57) ABSTRACT

The invention provides compounds of the general Formula (I)

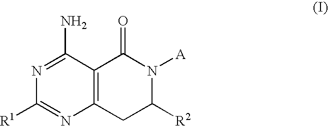

where R¹, R², and A are defined herein, as well as the preparation, compositions and uses thereof.

This application claims priority from U.S. Provisional Application No. 61/149,863, filed on Feb. 4, 2009; U.S. Provisional Application No. 61/150,028, filed on Feb. 5, 2009 and U.S. Provisional Application No. 61/285,408, filed on Dec. 10, 2009.

### FIELD OF THE INVENTION

The invention relates to 4-amino-7,8-dihydropyrido[4,3-d]pyrimidin-5(6 h)-one derivatives, as well as pharmaceutical compositions and uses thereof.

### BACKGROUND

It is estimated that somewhere between 34 and 61 million people in the US are obese and, in much of the developing world, incidence is increasing by about 1% per year. Obesity increases the likelihood of death from all causes by 20%, and more specifically, death from coronary artery disease and stroke are increased by 25% and 10%, respectively. Key priorities of anti-obesity treatments are to reduce food intake and/or hyperlipidemia. Since the latter has been suggested to provoke insulin resistance, molecules developed to prevent the accumulation of triglyceride would not only reduce obesity but they would also have the additional effect of reducing insulin resistance, a primary factor contributing to the development of diabetes. The therapeutic activity of leptin agonists has come under scrutiny through their potential to reduce food intake and, also, to reverse insulin resistance; however, their potential may be compromised by leptin-resistance, a characteristic of obesity. Acyl coenzyme A:diacylglycerol acyltransferase 1 (DGAT-1) is one of two known DGAT enzymes that catalyze the final step in mammalian triglyceride synthesis and an enzyme that is tightly implicated in both the development of obesity and insulin resistance. DGAT-1 deficient mice are resistant to diet-induced obesity through a mechanism involving increased energy expenditure. US researchers have now shown that these mice have decreased levels of tissue triglycerides, as well as increased sensitivity to insulin and to leptin. Importantly, DGAT-1 deficiency protects against insulin resistance and obesity in agouti yellow mice, a model of severe leptin resistance. Thus, DGAT-1 may represent a useful target for the treatment of insulin and leptin resistance and hence human obesity and diabetes. Chen, H. C., et al., J Clin Invest, 109(8), 1049-55 (2002).

Although studies show that DGAT-1 inhibition is useful for treating obesity and diabetes, there remains a need for DGAT-1 inhibitors that have efficacy for the treatment of metabolic disorders (e.g., obesity, Type 2 diabetes, and insulin resistance syndrome (also referred to as “metabolic syndrome”)).

### SUMMARY

The invention includes compounds of Formula (I)

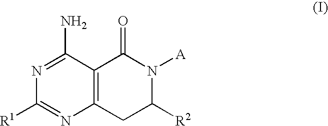

wherein

R¹ is hydrogen, (C₁-C₂)alkyl, or (C₁-C₂)alkoxy;

R² is hydrogen or (C₁-C₂)alkyl;

A is a group of formulae (1A), (1B), (1C) or (1D),

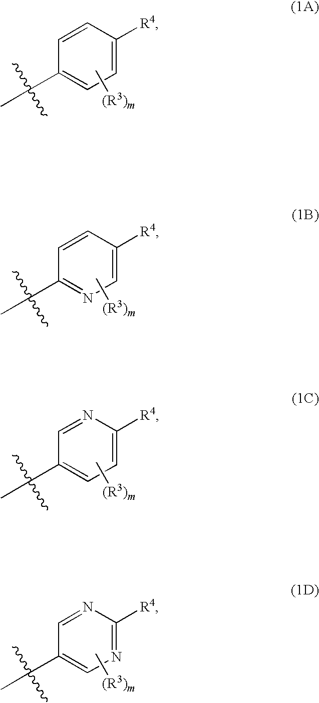

where each R³ is independently halogen, OH, (C₁-C₄)alkyl, cyano, (C₃-C₆)cycloalkyl or (C₁-C₄)alkoxy; and m is 0, 1, 2 or 3;

R⁴ is hydrogen, halogen, or a chemical moiety selected from the group consisting of:
(i) taken together with R³ to form a 5- to 6-membered carbocyclic fused ring, a 5- to 6-membered heterocyclic fused ring containing 1 to 2 heteroatoms each independently selected from O, N or S, or a 5- to 6-membered heteroaryl fused ring containing 1 to 2 heteroatoms each independently selected form O, N or S wherein the carbocyclic, heterocyclic and heteroaryl fused rings are optionally substituted with one to four substituents selected from the group consisting of (C₁-C₄)alkyl, (C₁-C₄)alkoxy, (C₁-C₄)haloalkyl, (C₁-C₄)haloalkoxy, -hydroxyl, halogen, cyano, oxo, —NH₂, —NH((C₁-C₄)alkyl), —N((C₁-C₄)alkyl)₂, —C(O)—OH, —C(O)—(C₁-C₄)alkoxy, —C(O)—NH₂, —C(O)—NH((C₁-C₄)alkyl), and —C(O)—N((C₁-C₄)alkyl)₂;
(ii) (C₁-C₆)alkyl optionally substituted with one or more substitutents selected from the group consisting of hydroxy, cyano, (C₁-C₆)alkoxy, halo-substituted (C₁-C₆)alkoxy, halogen, —NH₂, NH, oxo —S(C₁-C₄)alkyl, —SO(C₁-C₄)alkyl, —SO₂(C₁-C₄)alkyl, —O—SO₂(C₁-C₄)alkyl, —(CH₂)ₚC(O)—N(R^{6a})(R^{6b}), —(CH₂)ₚNH—C(O)(C₁-C₄)alkyl), —(CH₂)ₚNH—C(O)(C₁-C₄)alkoxy, —(CH₂)ₚC(O)—O(R^{6c}), —(CH₂)ₚOH, a 3- to 6-membered carbocyclic group, an aryl group, and a 5- to 6-membered heteroaryl group containing 1 to 4 heteroatoms each independently selected from O, S, and N, wherein the carbocyclic, aryl and heteroaryl groups are optionally substituted with (C₁-C₄)alkyl, —(CH₂)ₚC(O)—N(R^{6a})(R^{6b}), —(CH₂)ₚNH—C(O)(C₁-C₄)alkyl), —(CH₂)ₚC(O)—O(R^{6c}), —(CH₂)ₚNH—C(O)(C₁-C₄)alkoxy, or —(CH₂)ₚOH;
(iii) (C₁-C₆)alkoxy optionally substituted with one or more substitutents selected from the group consisting of hydroxy, cyano, (C₁-C₄)alkyl, halo-substituted (C₁-C₄)alkyl, halo-substituted (C₁-C₄)alkoxy, halogen, —NH₂, oxo or a 3- to 6-membered cycloalkyl group;
(iv) —S(C₁-C₄)alkyl, —SO(C₁-C₄)alkyl, or —SO₂(C₁-C₄)alkyl, or
(v) (CH₂)ₒ—C(O)—OR⁵, (CH₂)ₒ—C(O)—(C₁-C₄)alkoxy-R⁷, (CH₂)ₒ-C(O)—N(R⁵)(R⁶), or (CH₂)ₒ—C(O)—R⁵,
(vi) 3- to 6-membered carbocyclic ring or a 3- to 6-membered heterocyclic ring containing 1 to 2 heteroatoms each independently selected from O, N or S, wherein the carbocyclic and heterocyclic rings are optionally substituted with one to four substituents selected from the group consisting of —(CH₂)ₙC(O)—O(R⁵), —(CH₂)ₙOH, (C₁-C₄)alkoxy, —(CH₂)ₙC(O)—N(R⁵)(R⁶), —(CH₂)ₙOH, (C₁-C₄)alkyl, (C₁-C₄)haloalkyl, (C₁-C₄)haloalkoxy, hydroxyl, halogen, cyano, oxo, a 5- to 6-membered heteroaryl containing 1 to 3 heteroatoms each independently selected from O, N and S, and —N(R⁵)(R⁶), wherein said heteroaryl is optionally substituted with 1 to 3 substituents each independently selected from OH, halogen, or (C₁-C₄)alkyl;

n is 0 or 1;

o is 0, 1, or 2;

p is 0, 1, or 2;

R⁵, R⁶, R^{6a}, R^{6b}, and R^{6c} are each independently H or (C₁-C₄)alkyl; and

R⁷ is H, (C₁-C₄)alkyl, (C₃-C₆)cycloalkyl, or aryl;

or a pharmaceutically acceptable salt thereof.

Compounds of Formula (I) also include compounds wherein

R¹ is hydrogen, (C₁-C₂)alkyl, or (C₁-C₂)alkoxy;

R² is hydrogen or (C₁-C₂)alkyl;

A is a group of formulae (1A), (1B), (1C) or (1D),

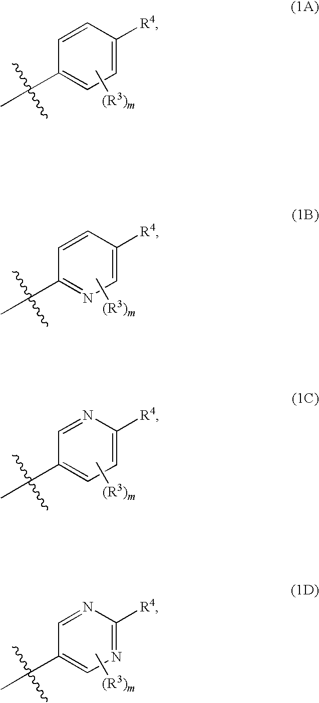

where R³ is (C₁-C₄)alkyl, (C₁-C₄)alkoxy, halo-substituted (C₁-C₄)alkyl, halo-substituted (C₁-C₄)alkoxy, halogen, or hydroxyl, or R³ is taken together with R⁴ to form a 5- to 6-membered carbocyclic fused ring or a 5- to 6-membered heterocyclic fused ring containing 1 to 2 heteroatoms selected from O, S or N;

m is 0 or 1;

R⁴ is hydrogen, halogen, or a chemical moiety selected from the group consisting of:
(i) taken together with R³ to form a 5- to 6-membered carbocyclic fused ring, a 5- to 6-membered heterocyclic fused ring containing 1 to 2 heteroatoms each independently selected from O, N or S, or a 5- to 6-membered heteroaryl fused ring containing 1 to 2 heteroatoms each independently selected form O, N or S;
(ii) (C₁-C₆)alkyl optionally substituted with hydroxy, cyano, (C₁-C₄)alkoxy, halo-substituted (C₁-C₄)alkyl, halo-substituted (C₁-C₄)alkoxy, or a 3- to 6-membered cycloalkyl group;
(iii) (C₁-C₆)alkoxy optionally substituted with a 3- to 6-membered cycloalkyl group;
(iv) —S(C₁-C₄)alkyl, —SO(C₁-C₄)alkyl, or —SO₂(C₁-C₄)alkyl;
(v) halo-substituted (C₁-C₄)alkyl;
(vi) halo-substituted (C₁-C₄)alkoxy;
(vii) 3- to 5-membered carbocyclic ring optionally substituted with —(CH₂)ₙC(O)—O(R⁶), —(CH₂)ₙOH, (C₁-C₄)alkoxy, cyano, or 1 to 2 halogens, where n is 0 or 1, and R⁵ is H or (C₁-C₄)alkyl,
(viii) —C(CH₃)₂—R⁶, where R⁶ is hydroxy, cyano, (C₁-C₆)alkoxy, halo-substituted (C₁-C₆)alkoxy, halogen, —NH₂, NH, oxo —S(C₁-C₄)alkyl, —SO(C₁-C₄)alkyl, —SO₂(C₁-C₄)alkyl, —O—SO₂(C₁-C₄)alkyl, —(CH₂)ₚC(O)—N(R^{6a})(R^{6b}), —(CH₂)ₚNH—C(O)(C₁-C₄)alkyl), —(CH₂)ₚNH—C(O)(C₁-C₄)alkoxy, —(CH₂)ₚC(O)—O(R^{6c}), —(CH₂)ₚOH, a 5- to 6-membered heteroaryl containing 1 to 4 heteroatoms each independently selected from oxygen, nitrogen or sulfur and optionally substituted with (C₁-C₄)alkyl, —(CH₂)ₚC(O)—N(R^{6a})(R^{6b}), —(CH₂)ₚNH—C(O)(C₁-C₄)alkyl), —(CH₂)ₚC(O)—O(R^{6c}), or —(CH₂)ₚOH—, where R^{6a}, R^{6b}, and R^{6c} are each independently selected from hydrogen or (C₁-C₄)alkyl and p is 0, 1, or 2;
(ix) —(CH₂)_{q}—C(OH)(R⁷)(R⁸), where q is 0 or 1 and R⁷ and R⁸ are each independently hydrogen, (C₁-C₄)alkyl, or a halo-substituted (C₁-C₄)alkyl;
(x) —(CH₂)ᵣ—C(O)—R⁹, where R⁹ is —NR^{9a}R^{9b} or —OR^{9c}, where r is 0 or 1, and R^{9a}, R^{9b} and R^{9c} are each independently selected form hydrogen or (C₁-C₄)alkyl; and
(xi) a group of formula (1E)

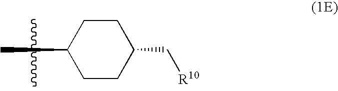

wherein R¹⁰ is
   (a) cyano;
   (b) —C(O)—N(R⁵)(R⁶);
   (c) —C(O)O(R⁵);
   (d) —(CH₂)ₙOH where n is 0, 1, or 2;
   (e) a 5- to 6-membered heteroaryl containing 1 to 3 heteroatoms each independently selected from oxygen, nitrogen or sulfur, wherein said heteroaryl is optionally substituted with 1 to 3 substituents each independently selected from OH, halogen, or (C₁-C₄)alkyl;
      or a pharmaceutically acceptable salt thereof.

Another aspect of the invention is a pharmaceutical composition that comprises (1) a compound of the invention, and (2) a pharmaceutically acceptable excipient, diluent, or carrier. The composition may comprise a therapeutically effective amount of a compound of the invention. The composition may also contain at least one additional pharmaceutical agent. Such agents include anti-obesity agents and/or anti-diabetic agents.

In yet another aspect of the invention, a method for treating a disease, disorder, or condition modulated by DGAT-1 inhibition in animals is provided that includes the step of administering to an animal, such as a human, in need of such treatment a therapeutically effective amount of a compound of the invention (or a pharmaceutical composition thereof). Diseases, conditions, and/or disorders mediated by DGAT-1 inhibition include, e.g., obesity (including weight control or weight maintenance), Type 2 diabetes, diabetic nephropathy, insulin resistance syndrome, hyperglycemia, hyperinsulinemia, hyperlipidemia, impaired glucose tolerance, hypertension, and reducing the level of blood glucose.

Compounds of the invention may be administered in combination with other pharmaceutical agents (in particular, anti-obesity and anti-diabetic agents described herein below). The combination therapy may be administered as (a) a single pharmaceutical composition which comprises a compound of the invention, at least one additional pharmaceutical agent described herein and a pharmaceutically acceptable excipient, diluent, or carrier; or (b) two separate pharmaceutical compositions comprising (i) a first composition comprising a compound of the invention and a pharmaceutically acceptable excipient, diluent, or carrier, and (ii) a second composition comprising at least one additional pharmaceutical agent described herein and a pharmaceutically acceptable excipient, diluent, or carrier. The pharmaceutical compositions may be administered simultaneously or sequentially and in any order.

It is to be understood that both the foregoing summary and the following detailed description and attendant claims are exemplary and explanatory only and are not restrictive of the invention, as claimed.

### DETAILED DESCRIPTION

The invention may be understood even more readily by reference to the following detailed description of exemplary embodiments of the invention and the examples included therein.

It is to be understood that this invention is not limited to specific synthetic methods of making that may of course vary. It is also to be understood that the terminology used herein is for the purpose of describing particular embodiments only and is not intended to be limiting. The plural and singular should be treated as interchangeable, other than the indication of number.

The headings within this document are only being utilized to expedite its review by the reader. They should not be construed as limiting the invention or claims in any manner.

In this specification and in the claims that follow, reference will be made to a number of terms that shall be defined to have the following meanings:

As used herein in the specification, “a” or “an” may mean one or more. As used herein in the claim(s), when used in conjunction with the word “comprising”, the words “a” or “an” may mean one or more than one. As used herein “another” may mean at least a second or more.

The term “about” refers to a relative term denoting an approximation of plus or minus 10% of the nominal value it refers, in one embodiment, to plus or minus 5%, in another embodiment, to plus or minus 2%. For the field of this disclosure, this level of approximation is appropriate unless the value is specifically stated require a tighter range.

As used herein, the term “alkyl” refers to a hydrocarbon radical of the general formula CₙH₂ₙ₊₁. The alkane radical may be straight or branched. For example, the term “(C₁-C₆)alkyl” refers to a monovalent, straight, or branched aliphatic group containing 1 to 6 carbon atoms (e.g., methyl, ethyl, n-propyl, i-propyl, n-butyl, i-butyl, s-butyl, t-butyl, n-pentyl, 1-methylbutyl, 2-methylbutyl, 3-methylbutyl, neopentyl, 3,3-dimethylpropyl, hexyl, 2-methylpentyl, and the like). Similarly, the alkyl portion (i.e., alkyl moiety) of an alkoxy group has the same definition as above. “Halo-substituted alkyl” or “halo-subsituted alkoxy” refers to an alkyl or alkoxy group substituted with one or more halogen atoms (e.g., fluoromethyl, difluoromethyl, trifluoromethyl, perfluoroethyl, 1,1-difluoroethyl and the like).

The term “cycloalkyl” refers to nonaromatic rings that are fully hydrogenated and may exist as a single ring, bicyclic ring or a spiral ring. Unless specified otherwise, the carbocyclic ring is generally a 3- to 6-membered ring. For example, cycloalkyl include groups such as cyclopropyl, cyclobutyl, cyclopentyl, cyclohexyl, cyclohexenyl, and the like.

“Halogen” or “halo” refers to refers to a chlorine, fluorine, iodine, or bromine atom.

The phrase “therapeutically effective amount” means an amount of a compound of the invention that (i) treats or prevents the particular disease, condition, or disorder, (ii) attenuates, ameliorates, or eliminates one or more symptoms of the particular disease, condition, or disorder, or (iii) prevents or delays the onset of one or more symptoms of the particular disease, condition, or disorder described herein.

The term “animal” refers to humans (male or female), companion animals (e.g., dogs, cats and horses), food-source animals, zoo animals, marine animals, birds and other similar animal species. “Edible animals” refers to food-source animals such as cows, pigs, sheep and poultry.

The phrase “pharmaceutically acceptable” indicates that the substance or composition must be compatible chemically and/or toxicologically, with the other ingredients comprising a formulation, and/or the mammal being treated therewith.

The terms “treating”, “treat”, or “treatment” embrace both preventative, i.e., prophylactic, and palliative treatment.

The terms “modulated” or “modulating”, or “modulate(s)”, as used herein, unless otherwise indicated, refers to the inhibition of the diacylglycerol O-acyltransferase 1 (DGAT-1) enzyme with compounds of the invention.

The terms “mediated” or “mediating” or “mediate(s)”, as used herein, unless otherwise indicated, refers to the treatment or prevention the particular disease, condition, or disorder, (ii) attenuation, amelioration, or elimination of one or more symptoms of the particular disease, condition, or disorder, or (iii) prevention or delay of the onset of one or more symptoms of the particular disease, condition, or disorder described herein, by inhibiting the DGAT-1 enzyme.

The terms “compounds (or compound) of the present application (or invention)” or simply “compounds” or “compound” (unless specifically identified otherwise) refer to compounds described herein and pharmaceutically acceptable salts thereof, encompassed within this application, such as compounds encompassed within general formulas and intermediates of the compounds as well as salts, all stereoisomers (including diastereoisomers and enantiomers), tautomers, conformational isomers, and isotopically labeled compounds. Hydrates and solvates of the compounds of the invention are considered to be part of the invention, wherein the compound is in association with water or solvent, respectively.

The term “salt” and “pharmaceutically acceptable salt” refers to inorganic and organic salts of a compound. These salts can be prepared in situ during the final isolation and purification of a compound, or by separately reacting the present compound with a suitable organic or inorganic acid or base and isolating the salt thus formed. Representative salts include the hydrobromide, hydrochloride, hydroiodide, sulfate, bisulfate, nitrate, acetate, trifluoroacetate, oxalate, besylate, palmitiate, pamoate, malonate, stearate, laurate, malate, borate, benzoate, lactate, phosphate, hexafluorophosphate, benzene sulfonate, tosylate, formate, citrate, maleate, fumarate, succinate, tartrate, naphthylate, mesylate, glucoheptonate, lactobionate, and laurylsulphonate salts, and the like. These may include cations based on the alkali and alkaline earth metals, such as sodium, lithium, potassium, calcium, magnesium, and the like, as well as non-toxic ammonium, quaternary ammonium, and amine cations including, but not limited to, ammonium, tetramethylammonium, tetraethylammonium, methylamine, dimethylamine, trimethylamine, triethylamine, ethylamine, and the like. See, e.g., Berge, et al., J. Pharm. Sci., 66, 1-19 (1977).

In one embodiment of the invention, R¹ is hydrogen, methoxy, or methyl; R² is hydrogen or methyl; R³ is halogen or (C₁-C₄)alkyl; and m is 0 or 1;

or a pharmaceutically acceptable salt thereof.
In another embodiment of the invention, A is a group of formula (1A),

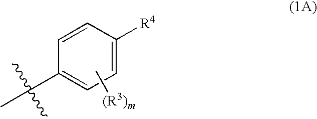

where R⁴ is (C₁-C₆)alkyl optionally substituted with one or more substitutents selected from the group consisting of hydroxy, cyano, (C₁-C₆)alkoxy, halo-substituted (C₁-C₆)alkoxy, halogen, —NH₂, NH, oxo —S(C₁-C₄)alkyl, —SO(C₁-C₄)alkyl, —SO₂(C₁-C₄)alkyl, —O—SO₂(C₁-C₄)alkyl, —(CH₂)ₚC(O)—N(R^{6a})(R^{6b}), —(CH₂)ₚNH—C(O)(C₁-C₄)alkyl), —(CH₂)ₚNH—C(O)(C₁-C₄)alkoxy, —(CH₂)ₚC(O)—O(R^{6c}), —(CH₂)ₚOH, a 3- to 6-membered carbocyclic group, an aryl group, and a 5- to 6-membered heteroaryl group containing 1 to 4 heteroatoms each independently selected from O, S, and N, wherein the carbocyclic, aryl and heteroaryl groups are optionally substituted with (C₁-C₄)alkyl, —(CH₂)ₚC(O)—N(R^{6a})(R^{6b}), —(CH₂)ₚNH—C(O)(C₁-C₄)alkyl), —(CH₂)ₚC(O)—O(R^{6c}), —(CH₂)ₚNH—C(O)(C₁-C₄)alkoxy, or —(CH₂)ₚOH;
or a pharmaceutically acceptable salt thereof.
In another embodiment of the invention R⁴ is (C₁-C₆)alkyl optionally substituted with one or more substitutents selected from the group consisting of hydroxy, cyano, (C₁-C₆)alkoxy, halo-substituted (C₁-C₆)alkoxy, halogen, —NH₂, NH, oxo, —(CH₂)ₚC(O)—N(R^{6a})(R^{6b}), and —(CH₂)ₚC(O)—O(R^{6c});
or a pharmaceutically acceptable salt thereof.
In another embodiment of the invention, wherein A is a group of formula (1A),

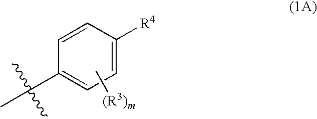

where R⁴ is a 3- to 6-membered carbocyclic ring or a 3- to 6-membered heterocyclic ring containing 1 to 2 heteroatoms each independently selected from O, N or S, wherein the carbocyclic and heterocyclic rings are optionally substituted with one to four substituents selected from the group consisting of —(CH₂)ₙC(O)—O(R⁵), —(CH₂)ₙOH, (C₁-C₄)alkoxy, —(CH₂)ₙC(O)—N(R⁵)(R⁶), —(CH₂)ₙOH, (C₁-C₄)alkyl, (C₁-C₄)haloalkyl, (C₁-C₄)haloalkoxy, hydroxyl, halogen, cyano, oxo, a 5- to 6-membered heteroaryl containing 1 to 3 heteroatoms each independently selected from O, N and S, and —N(R⁵)(R⁶), wherein said heteroaryl is optionally substituted with 1 to 3 substituents each independently selected from OH, halogen, or (C₁-C₄)alkyl;
or a pharmaceutically acceptable salt thereof.

In another embodiment of the invention, R⁴ is a 3- to 6-membered carbocyclic ring optionally substituted with one or two substituents selected from the group consisting of —(CH₂)ₙC(O)—O(R⁵), —(CH₂)ₙOH, (C₁-C₄)alkoxy, —(CH₂)ₙC(O)—N(R⁵)(R⁶), (C₁-C₄)alkyl, (C₁-C₄)haloalkyl, (C₁-C₄)haloalkoxy, hydroxyl, halogen, cyano, and oxo;

or a pharmaceutically acceptable salt thereof.

Another embodiment of the invention is directed at a pharmaceutical composition comprising (i) a compound of any one of the preceding claims; and (ii) a pharmaceutically acceptable excipient, diluent, or carrier.

In another embodiment, the compound or said pharmaceutically acceptable salt thereof is present in a therapeutically effective amount.

In yet another embodiment of the invention. The composition further comprises at least one additional pharmaceutical agent selected from the group consisting of an anti-obesity agent and an anti-diabetic agent..

In yet another embodiment of the invention, said anti-obesity agent is selected from the group consisting of dirlotapide, mitratapide, implitapide, R56918 (CAS No. 403987), CAS No. 913541-47-6, lorcaserin, cetilistat, PYY₃₋₃₆, naltrexone, oleoyl-estrone, obinepitide, pramlintide, tesofensine, leptin, liraglutide, bromocriptine, orlistat, exenatide, AOD-9604 (CAS No. 221231-10-3) and sibutramine; and said anti-diabetic agent is selected from the group consisting of metformin, acetohexamide, chlorpropamide, diabinese, glibenclamide, glipizide, glyburide, glimepiride, gliclazide, glipentide, gliquidone, glisolamide, tolazamide, tolbutamide, tendamistat, trestatin, acarbose, adiposine, camiglibose, emiglitate, miglitol, voglibose, pradimicin-Q, salbostatin, balaglitazone, ciglitazone, darglitazone, englitazone, isaglitazone, pioglitazone, rosiglitazone, troglitazone, exendin-3, exendin-4, trodusquemine, reservatrol, hyrtiosal extract, sitagliptin, vildagliptin, alogliptin and saxagliptin.

Another embodiment of the invention is directed at a method for treating or delaying the progression or onset of Type 2 diabetes and diabetes-related disorders in animals comprising the step of administering to an animal in need of such treatment a therapeutically effective amount of a compound of the invention.

In another embodiment, the method for treating or delaying the progression or onset of Type 2 diabetes and diabetes-related disorders in animals comprises the step of administering to an animal in need of such treatment a pharmaceutical composition of the invention.

In another embodiment includes a method for treating a disease, condition or disorder modulated by the inhibition of DGAT-1 in animals comprising the step of administering to an animal in need of such treatment two separate pharmaceutical compositions comprising
(i) a first composition comprising a compound of the invention, and a pharmaceutically acceptable excipient, diluent, or carrier; and
(ii) a second composition comprising at least one additional pharmaceutical agent selected from the group consisting of an anti-obesity agent and an anti-diabetic agent, and a pharmaceutically acceptable excipient, diluent, or carrier;
   wherein said disease, condition or disorder modulated by the inhibition of DGAT-1 is selected from the group consisting of obesity, obesity-related disorders, Type 2 diabetes, and diabetes-related disorders; wherein said first composition and said second composition are administered simultaneously or sequentially and in any order.

Another embodiment of the invention includes the use of a compound of the invention or a pharmaceutically acceptable salt thereof in the manufacture of a medicament for treating a disease, condition or disorder that is modulated by the inhibition of DGAT-1.

The invention also includes solvates and hydrates of the compounds of the invention. The term “solvate” refers to a molecular complex of a compound of this invention (including pharmaceutically acceptable salts thereof) with one or more solvent molecules. Such solvent molecules are those commonly used in the pharmaceutical art, which are known to be innocuous to the recipient, e.g., water, ethanol, ethylene glycol, and the like, The term “hydrate” refers to the complex where the solvent molecule is water. The solvates and/or hydrates may exist in crystalline form. Other solvents may be used as intermediate solvates in the preparation of more desirable solvates, such as methanol, methyl t-butyl ether, ethyl acetate, methyl acetate, (S)-propylene glycol, (R)-propylene glycol, 1,4-butyne-diol, and the like.

The compounds of the invention may contain asymmetric or chiral centers, and, therefore, exist in different stereoisomeric forms. Unless specified otherwise, it is intended that all stereoisomeric forms of the compounds of the invention as well as mixtures thereof, including racemic mixtures, form part of the invention. In addition, the invention embraces all geometric and positional isomers. For example, if a compound of the invention incorporates a double bond or a fused ring, both the cis- and trans- forms, as well as mixtures, are embraced within the scope of the invention.

Diastereomeric mixtures can be separated into their individual diastereoisomers on the basis of their physical chemical differences by methods well known to those skilled in the art, such as by chromatography and/or fractional crystallization. Enantiomers can be separated by converting the enantiomeric mixture into a diastereomeric mixture by reaction with an appropriate optically active compound (e.g., chiral auxiliary such as a chiral alcohol or Mosher's acid chloride), separating the diastereoisomers and converting (e.g., hydrolyzing) the individual diastereoisomers to the corresponding pure enantiomers. Also, some of the compounds of the invention may be atropisomers (e.g., substituted biaryls) and are considered as part of this invention. Enantiomers can also be separated by use of a chiral HPLC column. Alternatively, the specific stereoisomers may be synthesized by using an optically active starting material, by asymmetric synthesis using optically active reagents, substrates, catalysts or solvents, or by converting one stereoisomer into the other by asymmetric transformation.

It is also possible that the intermediates and compounds of the invention may exist in different tautomeric forms, and all such forms are embraced within the scope of the invention. The term “tautomer” or “tautomeric form” refers to structural isomers of different energies which are interconvertible via a low energy barrier. For example, proton tautomers (also known as prototropic tautomers) include interconversions via migration of a proton, such as keto-enol and imine-enamine isomerizations. A specific example of a proton tautomer is the imidazole moiety where the proton may migrate between the two ring nitrogens. Valence tautomers include interconversions by reorganization of some of the bonding electrons.

Certain compounds of the invention may exist in different stable conformational forms which may be separable. Torsional asymmetry due to restricted rotation about an asymmetric single bond, for example, because of steric hindrance or ring strain, may permit separation of different conformers.

The invention also embraces isotopically-labeled compounds of the invention which are identical to those recited herein, but for the fact that one or more atoms are replaced by an atom having an atomic mass or mass number different from the atomic mass or mass number usually found in nature. Examples of isotopes that can be incorporated into compounds of the invention include isotopes of hydrogen, carbon, nitrogen, oxygen, phosphorus, sulfur, fluorine, iodine, and chlorine, such as ²H, ³H, ¹¹C, ¹³C, ¹⁴C, ¹³N, ¹⁵N, ¹⁵O, ¹⁷O, ¹⁸O, ³¹P, ³²P, ³⁵S, ¹⁸F, ¹²³I, ¹²⁵I and ³⁶Cl, respectively.

Certain isotopically-labeled compounds of the invention (e.g., those labeled with ³H and ¹⁴C) are useful in compound and/or substrate tissue distribution assays. Tritiated (i.e., ³H) and carbon-14 (i.e., ¹⁴C) isotopes may be used for their ease of preparation and detectability. Further, substitution with heavier isotopes such as deuterium (i.e., ²H) may afford certain therapeutic advantages resulting from greater metabolic stability (e.g., increased in vivo half-life or reduced dosage requirements) and hence may be used in some circumstances. Positron emitting isotopes such as ¹⁵O, ¹³N, ¹¹C, and ¹⁸F are useful for positron emission tomography (PET) studies to examine substrate occupancy. Isotopically labeled compounds of the invention can generally be prepared by following procedures analogous to those disclosed in the Schemes and/or in the Examples herein below, by substituting an isotopically labeled reagent for a non-isotopically labeled reagent.

Certain compounds of the invention may exist in more than one crystal form (generally referred to as “polymorphs”). Polymorphs may be prepared by crystallization under various conditions, for example, using different solvents or different solvent mixtures for recrystallization; crystallization at different temperatures; and/or various modes of cooling, ranging from very fast to very slow cooling during crystallization. Polymorphs may also be obtained by heating or melting the compound of the invention followed by gradual or fast cooling. The presence of polymorphs may be determined by solid probe NMR spectroscopy, IR spectroscopy, differential scanning calorimetry, powder X-ray diffraction or such other techniques.

In general, compounds of this invention may be prepared by methods that include processes known in the chemical arts, particularly in light of the description contained herein in combination with the knowledge of the skilled artisan. Although other reagents, starting materials, intermediate compounds or methods can be used in practice or testing, generalized methods for the preparation of the compounds of Formula I are illustrated by the following descriptions, Preparations, and reaction Schemes. Other preparation methods are described in the experimental section. The methods disclosed herein, including those outlined in the Schemes, Preparations, and Examples are for intended for illustrative purposes and are not to be construed in any manner as limitations thereon.

The starting materials are generally available from commercial sources such as Aldrich Chemicals (Milwaukee, Wis.) or are readily prepared using methods well known to those skilled in the art (e.g., prepared by methods generally described in Louis F. Fieser and Mary Fieser, Reagents for Organic Synthesis, v. 1-19, Wiley, New York (1967-1999 ed.), or Beilsteins Handbuch der organischen Chemie, 4, Aufl. ed. Springer-Verlag, Berlin, including supplements (also available via the Beilstein online database)).

Those skilled in the art will appreciate that other synthetic routes may be used to synthesize the inventive compounds. Although specific starting materials and reagents are depicted in the schemes and discussed below, other starting materials and reagents can be easily substituted to provide a variety of derivatives and/or reaction conditions. In addition, many of the compounds prepared by the methods described below can be further modified in light of this disclosure using conventional chemistry well known to those skilled in the art.

Compounds of the invention may be synthesized by synthetic routes that include processes analogous to those well-known in the chemical arts, particularly in light of the description contained herein. The starting materials are generally available from commercial sources such as Aldrich Chemicals (Milwaukee, Wis.) or are readily prepared using methods well known to those skilled in the art (e.g., prepared by methods generally described in Louis F. Fieser and Mary Fieser, Reagents for Organic Synthesis, v. 1-19, Wiley, New York (1967-1999 ed.), or Beilsteins Handbuch der organischen Chemie, 4, Aufl. ed. Springer-Verlag, Berlin, including supplements (also available via the Beilstein online database)).

For illustrative purposes, the reaction schemes depicted below provide potential routes for synthesizing the compounds of the invention as well as key intermediates. For a more detailed description of the individual reaction steps, see the Examples section below. Those skilled in the art will appreciate that other synthetic routes may be used to synthesize the inventive compounds. Although specific starting materials and reagents are depicted in the schemes and discussed below, other starting materials and reagents can be easily substituted to provide a variety of derivatives and/or reaction conditions. In addition, many of the compounds prepared by the methods described below can be further modified in light of this disclosure using conventional chemistry well known to those skilled in the art.

In the preparation of compounds of the invention, protection of remote functionality (e.g., primary or secondary amine) of intermediates may be necessary. The need for such protection will vary depending on the nature of the remote functionality and the conditions of the preparation methods. Suitable amino-protecting groups (NH-Pg) include acetyl, trifluoroacetyl, t-butoxycarbonyl (BOC), benzyloxycarbonyl (CBz) and 9-fluorenylmethyleneoxycarbonyl (Fmoc). The need for such protection is readily determined by one skilled in the art. For a general description of protecting groups and their use, see T. W. Greene, Protective Groups in Organic Synthesis, John Wiley & Sons, New York, 1991.

Scheme I outlines some general procedures that one could use to provide compounds of Formula (I).

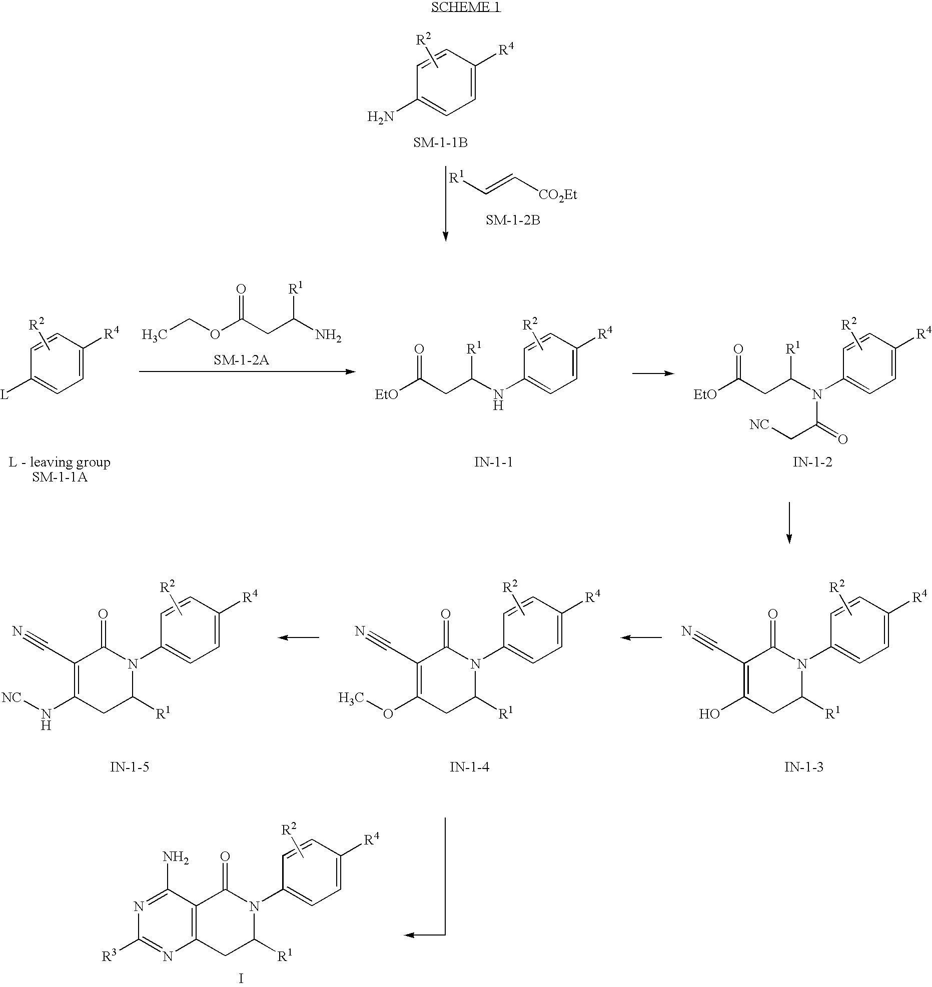

Desired starting materials (SM-1-1A, SM-1-1B, SM-1-2A and SM-1-2B) may be purchased from commercial sources or made using procedures known in the art. Starting materials (SM-1-2A and SM-1-2B) where R¹ is not H may be made or purchased as racemic mixtures or, if desired, as single enantiomers.

Aryl ester (IN-1-1) can be prepared by coupling together the desired starting materials (SM-1-1A (where L is a leaving group such as halogen, triflate, tosylate, etc.), SM-1-1B) at elevated temperatures (e.g., about 80° C. to about 130° C.) in the presence of a Palladium (or copper) catalyst, a weak base (e.g., cesium carbonate), and 2-dicyclohexyl phosphino-2′,4′,6′-triisopropylbiphenyl (X-PHOS) in an inert environment. Alternatively, aryl amine (SM-1-1B) and aryl ester (IN-1-1) may be coupled in the presence of a base, such as triethyl amine (TEA) in an appropriate solvent such as ethanol to afford the aryl ester (IN-1-1). Preferably, the reaction is conducted at elevated temperatures. Cyanoacetic acid can then be added to the aryl amine via an amide coupling reaction using procedures well known in the art. For example, cyanoacetic acid may be added in the presence of an activator such as N-N′-diisopropylcarbodiimide (DIC) or (2-(7-aza-1H-benzotriazole-1-yl)-1,1,3,3-tetramethyluronium hexafluorophosphate) (HATU) and a mild base, such as 4-dimethylaminopyridine (DMAP) in an appropriate solvent such as N-N-dimethyl formamide (DIMETHYLFORMAMIDE) to provide the corresponding cyanoamide intermediate (IN-1-2). Formation of the lactam (IN-1-3) can be achieved by treatment with base, such as 1,8-diazabicycloundec-7-ene (DBU), in methanol. Preferably, this reaction is conducted at elevated temperatures. Methylation of the lactam intermediate can be accomplished via the addition of oxalyl chloride in the presence of dichloromethane (DCM) and dimethylsulfoxide (DMSO) at low temperature, followed by the addition of methanol. The resulting methoxy lactam intermediate (IN-1-4) may then be reacted with cyanamide in the presence of sodium methoxide and methanol to provide the corresponding aminonitrile intermediate (IN-1-5). Cyclization is affected via treatment with a strong mineral acid, e.g., sulfuric acid, in an alcohol solvent, e.g., methanol (MeOH), preferably at elevated temperatures, to form the desired aminopyrimidine of Formula (I).

Alternatively, the methoxy lactam (IN-1-4) may be treated with the desired amidine in the presence of a base, e.g. diisopropylethyl amine (DIPEA) in an appropriate solvent, e.g. methanol, to provide the corresponding aminopyrimidine of Formula (I).

Scheme II outlines the general procedures one could use to provide compounds of the general Formula (II).

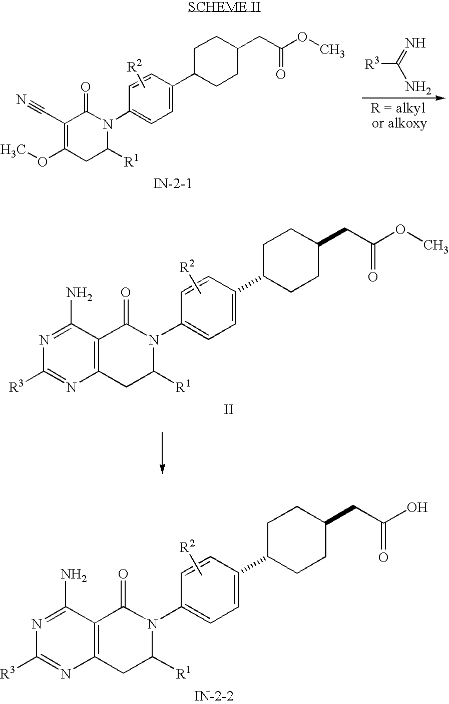

In Scheme II, treatment of a methyoxy lactam intermediate (IN-2-1) with a substituted amidine in the presence of base, e.g. diisopropylethyl amine (DIPEA) in an appropriate solvent, e.g. methanol, affords the corresponding aminopyrimidine of the general Formula II. Compounds of Formula II can be hydrolyzed using a strong base such as potassium hydroxide in the presence of water and the appropriate solvent or mixture of solvents, e.g. tetrahydrofuran (THF) and methanol (MeOH) to afford a carboxylic acid (IN-2-2), which in turn may be converted to other compounds of the invention as shown below in Scheme III.

Scheme III outlines the general procedure used to prepare compounds of the general Formulae (III), (IV) and (V).

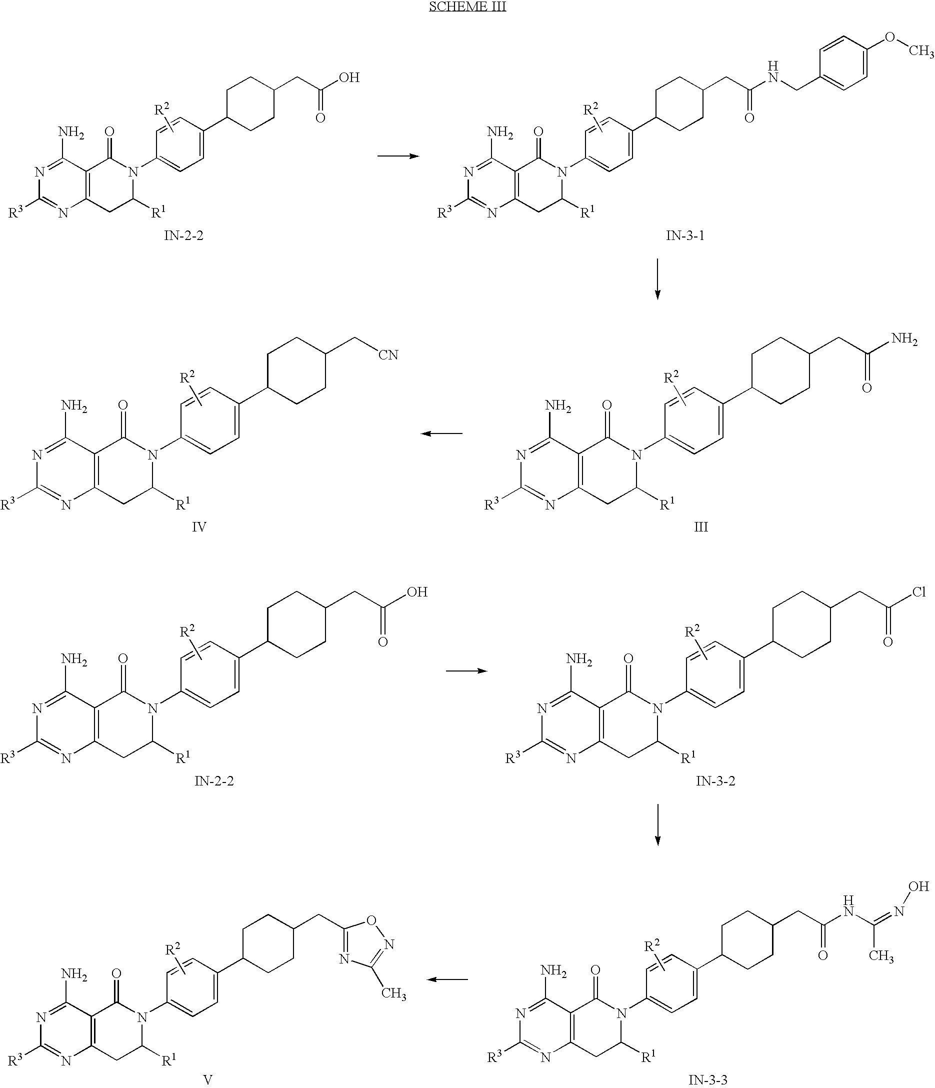

Compounds of Formulae (III), (IV), and (V) may be generally derived from intermediate compounds (IN-2-2). As shown in Scheme III, the aminopyrimidine intermediate can be coupled with (4-methoxyphenyl)methanamine using a coupling reagent such as benzotriazole-1-yl-oxy-tris-(dimethylamino)-phosphonium hexafluorophosphate (BOP) in an appropriate solvent such as N-N-dimethyl formamide to provide the corresponding amide intermediate (IN-3-1). Treatment with trifluoroacetic acid (TFA), preferably at elevated temperatures, affords compounds of the general Formula (III). Alternatively, compounds of Formula (III) may be prepared via the acid halide (IN-3-2) whereby the acid intermediate (IN-2-2) is treated with oxalyl chloride in an appropriate solvent or mixture of solvents, preferably at low temperature, followed by the addition of ammonia in dioxane (shown in Scheme IV).

Compounds of the general Formula (III) can be converted to compounds of the general Formula (IV) by treatment with phosphorus oxychloride (POCl₃). Preferably, this reaction is conducted at elevated temperatures.

As also shown in Scheme III, acid intermediates (IN-2-2) can be converted to the corresponding acid halide (IN-3-2) using thionyl chloride (SOCl₂) and catalytic N-N-dimethyl formamide (DMF). Treatment of the acid halide with hydroxyacetamidine provides the corresponding hydroxyiminoacetamide intermediate (IN-3-3) which is then heated in the presence of dimethyl amine (DMA) to give compounds of the general Formula (V). Preferably this reaction is conducted at high temperatures, such as between 100° C. to 140° C.

Scheme IV outlines a general procedure for the preparation of compounds of the general Formula VI.

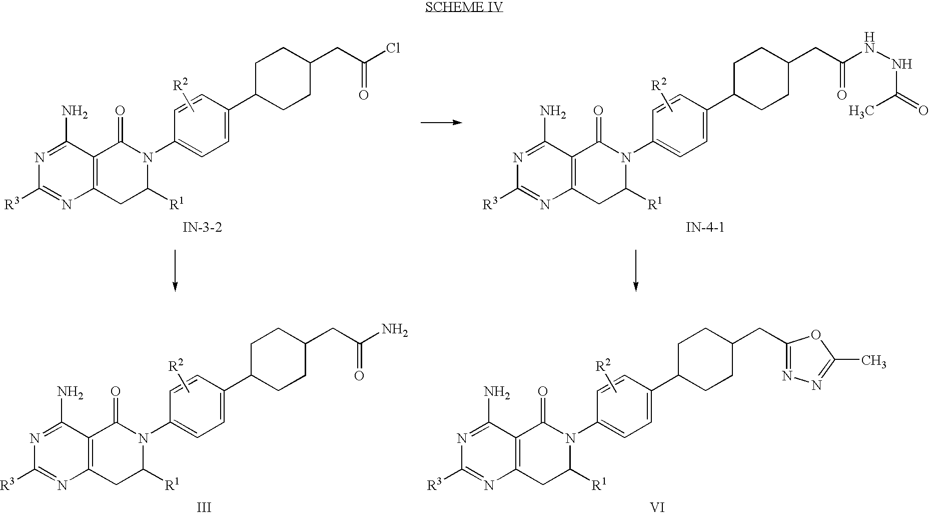

Compounds of the general Formula (VI) may be generally derived from intermediate compounds (IN-3-2). As shown in Scheme IV, treatment of the acid chloride with acetohydrazide provides the corresponding intermediate (IN-4-1). Cyclization via the addition of triphenyl phosphine (PPh₃), iodine (I₂) in the presence of a base such as triethyl amine (NEt₃) in an appropriate solvent, such as dichloromethane (DCM) provides compounds of the general Formula (VI).

Compounds of the invention are useful for treating diseases, conditions and/or disorders modulated by the inhibition of the DGAT-1 enzyme; therefore, another embodiment of the invention is a pharmaceutical composition comprising a therapeutically effective amount of a compound of the invention and a pharmaceutically acceptable excipient, diluent or carrier. The compounds of the invention (including the compositions and processes used therein) may also be used in the manufacture of a medicament for the therapeutic applications described herein.

A typical formulation is prepared by mixing a compound of the invention and a carrier, diluent or excipient. Suitable carriers, diluents and excipients are well known to those skilled in the art and include materials such as carbohydrates, waxes, water soluble and/or swellable polymers, hydrophilic or hydrophobic materials, gelatin, oils, solvents, water, and the like. The particular carrier, diluent or excipient used will depend upon the means and purpose for which the compound of the invention is being applied. Solvents are generally selected based on solvents recognized by persons skilled in the art as safe (GRAS) to be administered to a mammal. In general, safe solvents are non-toxic aqueous solvents such as water and other non-toxic solvents that are soluble or miscible in water. Suitable aqueous solvents include water, ethanol, propylene glycol, polyethylene glycols (e.g., PEG400, PEG300), etc. and mixtures thereof. The formulations may also include one or more buffers, stabilizing agents, surfactants, wetting agents, lubricating agents, emulsifiers, suspending agents, preservatives, antioxidants, opaquing agents, glidants, processing aids, colorants, sweeteners, perfuming agents, flavoring agents and other known additives to provide an elegant presentation of the drug (i.e., a compound of the invention or pharmaceutical composition thereof) or aid in the manufacturing of the pharmaceutical product (i.e., medicament).

The formulations may be prepared using conventional dissolution and mixing procedures. For example, the bulk drug substance (i.e., compound of the invention or stabilized form of the compound (e.g., complex with a cyclodextrin derivative or other known complexation agent)) is dissolved in a suitable solvent in the presence of one or more of the excipients described above. The compound of the invention is typically formulated into pharmaceutical dosage forms to provide an easily controllable dosage of the drug and to give the patient an elegant and easily handleable product.

The pharmaceutical composition (or formulation) for application may be packaged in a variety of ways depending upon the method used for administering the drug. Generally, an article for distribution includes a container having deposited therein the pharmaceutical formulation in an appropriate form. Suitable containers are well-known to those skilled in the art and include materials such as bottles (plastic and glass), sachets, ampoules, plastic bags, metal cylinders, and the like. The container may also include a tamper-proof assemblage to prevent indiscreet access to the contents of the package. In addition, the container has deposited thereon a label that describes the contents of the container. The label may also include appropriate warnings.

The invention further provides a method of treating diseases, conditions and/or disorders modulated by the inhibition of the DGAT-1 enzyme in an animal that includes administering to an animal in need of such treatment a therapeutically effective amount of a compound of the invention or a pharmaceutical composition comprising an effective amount of a compound of the invention and a pharmaceutically acceptable excipient, diluent, or carrier. The method is particularly useful for treating diseases, conditions and/or disorders that benefit from the inhibition of DGAT-1.

One aspect of the invention is the treatment of obesity, and obesity-related disorders (e.g., overweight, weight gain, or weight maintenance).

Obesity and overweight are generally defined by body mass index (BMI), which is correlated with total body fat and estimates the relative risk of disease. BMI is calculated by weight in kilograms divided by height in meters squared (kg/m²). Overweight is typically defined as a BMI of 25-29.9 kg/m², and obesity is typically defined as a BMI of 30 kg/m². See, e.g., National Heart, Lung, and Blood Institute, Clinical Guidelines on the Identification, Evaluation, and Treatment of Overweight and Obesity in Adults, The Evidence Report, Washington, D.C.: U.S. Department of Health and Human Services, NIH publication no. 98-4083 (1998).

Another aspect of the invention is for the treatment or delaying the progression or onset of diabetes or diabetes-related disorders including Type 1 (insulin-dependent diabetes mellitus, also referred to as “IDDM”) and Type 2 (noninsulin-dependent diabetes mellitus, also referred to as “NIDDM”) diabetes, impaired glucose tolerance, insulin resistance, hyperglycemia, and diabetic complications (such as atherosclerosis, coronary heart disease, stroke, peripheral vascular disease, nephropathy, hypertension, neuropathy, and retinopathy).

Yet another aspect of the invention is the treatment of diabetes- or obesity-related co-morbidities, such as metabolic syndrome. Metabolic syndrome includes diseases, conditions or disorders such as dyslipidemia, hypertension, insulin resistance, diabetes (e.g., Type 2 diabetes), weight gain, coronary artery disease and heart failure. For more detailed information on Metabolic Syndrome, see, e.g., Zimmet, P.Z., et al., “The Metabolic Syndrome: Perhaps an Etiologic Mystery but Far From a Myth —Where Does the International Diabetes Federation Stand?,” Diabetes & Endocrinology, 7(2), (2005); and Alberti, K. G., et al., “The Metabolic Syndrome —A New Worldwide Definition,” Lancet, 366, 1059-62 (2005). Administration of the compounds of the invention may provide a statistically significant (p<0.05) reduction in at least one cardiovascular disease risk factor, such as lowering of plasma leptin, C-reactive protein (CRP) and/or cholesterol, as compared to a vehicle control containing no drug. The administration of compounds of the invention may also provide a statistically significant (p<0.05) reduction in glucose serum levels.

In yet another aspect of the invention, the condition treated is impaired glucose tolerance, hyperglycemia, diabetic complications such as sugar cataracts, diabetic neuropathy, diabetic nephropathy, diabetic retinopathy and diabetic cardiomyopathy, anorexia nervosa, bulimia, cachexia, hyperuricemia, hyperinsulinemia, hypercholesterolemia, hyperlipidemia, dyslipidemia, mixed dyslipidemia, hypertriglyceridemia, nonalcoholic fatty liver disease, atherosclerosis, arteriosclerosis, acute heart failure, congestive heart failure, coronary artery disease, cardiomyopathy, myocardial infarction, angina pectoris, hypertension, hypotension, stroke, ischemia, ischemic reperfusion injury, aneurysm, restenosis, vascular stenosis, solid tumors, skin cancer, melanoma, lymphoma, breast cancer, lung cancer, colorectal cancer, stomach cancer, esophageal cancer, pancreatic cancer, prostate cancer, kidney cancer, liver cancer, bladder cancer, cervical cancer, uterine cancer, testicular cancer and ovarian cancer.

The invention also relates to therapeutic methods for treating the above described conditions in a mammal, including a human, wherein a compound of this invention is administered as part of an appropriate dosage regimen designed to obtain the benefits of the therapy. The appropriate dosage regimen, the amount of each dose administered and the intervals between doses of the compound will depend upon the compound of this invention being used, the type of pharmaceutical compositions being used, the characteristics of the subject being treated and the severity of the conditions.

The invention also provides pharmaceutical compositions which comprise a therapeutically effective amount of a compound, or a pharmaceutically acceptable salt thereof, in admixture with at least one pharmaceutically acceptable excipient. The compositions include those in a form adapted for oral, topical or parenteral use and can be used for the treatment of diabetes and related conditions as described above.

The composition can be formulated for administration by any route known in the art, such as subdermal, inhalation, oral, topical, parenteral, etc. The compositions may be in any form known in the art, including but not limited to tablets, capsules, powders, granules, lozenges, or liquid preparations, such as oral or sterile parenteral solutions or suspensions.

Tablets and capsules for oral administration may be in unit dose presentation form, and may contain conventional excipients such as binding agents, for example syrup, acacia, gelatin, sorbitol, tragacanth, or polyvinylpyrollidone; fillers, for example lactose, sugar, maize-starch, calcium phosphate, sorbitol or glycine; tabletting lubricants, for example magnesium stearate, talc, polyethylene glycol or silica; disintegrants, for example potato starch; or acceptable wetting agents such as sodium lauryl sulphate. The tablets may be coated according to methods well known in normal pharmaceutical practice.

Oral liquid preparations may be in the form of, for example, aqueous or oily suspensions, solutions, emulsions, syrups or elixirs, or may be presented as a dry product for reconstitution with water or other suitable vehicle before use. Such liquid preparations may contain conventional additives, such as suspending agents, for example sorbitol, methyl cellulose, glucose syrup, gelatin, hydroxyethyl cellulose, carboxymethyl cellulose, aluminium stearate gel or hydrogenated edible fats, emulsifying agents, for example lecithin, sorbitan monooleate, or acacia; non-aqueous vehicles (which may include edible oils), for example almond oil, oily esters such as glycerin, propylene glycol, or ethyl alcohol; preservatives, for example methyl or propyl p-hydroxybenzoate or sorbic acid, and, if desired, conventional flavoring or coloring agents.

For parenteral administration, fluid unit dosage forms are prepared utilizing the compound and a sterile vehicle, water being preferred. The compound, depending on the vehicle and concentration used, can be either suspended or dissolved in the vehicle or other suitable solvent. In preparing solutions, the compound can be dissolved in water for injection and filter sterilized before filling into a suitable vial or ampoule and sealing. Advantageously, agents such as local anesthetics, preservatives and buffering agents etc. can be dissolved in the vehicle. To enhance the stability, the composition can be frozen after filling into the vial and the water removed under vacuum. The dry lyophilized powder is then sealed in the vial and an accompanying vial of water for injection may be supplied to reconstitute the liquid prior to use. Parenteral suspensions are prepared in substantially the same manner except that the compound is suspended in the vehicle instead of being dissolved and sterilization cannot be accomplished by filtration. The compound can be sterilized by exposure to ethylene oxide before suspending in the sterile vehicle. Advantageously, a surfactant or wetting agent is included in the composition to facilitate uniform distribution of the compound.

The compositions may contain, for example, from about 0.1% to about 99 by weight, of the active material, depending on the method of administration. Where the compositions comprise dosage units, each unit will contain, for example, from about 0.1 to 900 mg of the active ingredient, more typically from 1 mg to 250 mg, or 0.01 mg/kg/day to 30 mg/kg/day, such as 0.01 mg/kg/day to 5 mg/kg/day of active compound in single or divided doses.

Compounds of the invention can be formulated for administration in any convenient way for use in human or veterinary medicine, by analogy with other anti-diabetic agents. Such methods are known in the art and have been summarized above. For a more detailed discussion regarding the preparation of such formulations; the reader's attention is directed to Reminaton's Pharmaceutical Sciences, 21^{st} Edition, by University of the Sciences in Philadelphia.

It is also noted that the compounds of the invention can be used in sustained release, controlled release, and delayed release formulations, which forms are also well known to one of ordinary skill in the art.

The compounds of this invention may also be used in conjunction with other pharmaceutical agents for the treatment of the diseases, conditions and/or disorders described herein. Therefore, methods of treatment that include administering compounds of the invention in combination with other pharmaceutical agents are also provided. Suitable pharmaceutical agents that may be used in combination with the compounds of the invention include anti-obesity agents (including appetite suppressants), anti-diabetic agents, anti-hyperglycemic agents, lipid lowering agents, and anti-hypertensive agents.

Suitable anti-diabetic agents include an acetyl-CoA carboxylase-2 (ACC-2) inhibitor, a phosphodiesterase (PDE)-10 inhibitor, a sulfonylurea (e.g., acetohexamide, chlorpropamide, diabinese, glibenclamide, glipizide, glyburide, glimepiride, gliclazide, glipentide, gliquidone, glisolamide, tolazamide, and tolbutamide), a meglitinide, an α-amylase inhibitor (e.g., tendamistat, trestatin and AL-3688), an α-glucoside hydrolase inhibitor (e.g., acarbose), an α-glucosidase inhibitor (e.g., adiposine, camiglibose, emiglitate, miglitol, voglibose, pradimicin-Q, and salbostatin), a PPARγ agonist (e.g., balaglitazone, ciglitazone, darglitazone, englitazone, isaglitazone, pioglitazone, rosiglitazone and troglitazone), a PPAR α/γ agonist (e.g., CLX-0940, GW-1536, GW-1929, GW-2433, KRP-297, L-796449, LR-90, MK-0767 and SB-219994), a biguanide (e.g., metformin), a glucagon-like peptide 1 (GLP-1) agonist (e.g., exendin-3 and exendin-4), a protein tyrosine phosphatase-1B (PTP-1B) inhibitor (e.g., trodusquemine, hyrtiosal extract, and compounds disclosed by Zhang, S., et al., Drug Discovery Today, 12(9/10), 373-381 (2007)), SIRT-1 inhibitor (e.g., reservatrol), a dipeptidyl peptidease IV (DPP-IV) inhibitor (e.g., sitagliptin, vildagliptin, alogliptin and saxagliptin), an insulin secreatagogue, a fatty acid oxidation inhibitor, an A2 antagonist, a c-jun amino-terminal kinase (JNK) inhibitor, insulin, an insulin mimetic, a glycogen phosphorylase inhibitor, a VPAC2 receptor agonist and a glucokinase activator. Exemplary anti-diabetic agents are metformin and DPP-IV inhibitors (e.g., sitagliptin, vildagliptin, alogliptin and saxagliptin).

Suitable anti-obesity agents include 11β-hydroxy steroid dehydrogenase-1 (11β-HSD type 1) inhibitors, stearoyl-CoA desaturase-1 (SCD-1) inhibitor, MCR-4 agonists, cholecystokinin-A (CCK-A) agonists, monoamine reuptake inhibitors (such as sibutramine), sympathomimetic agents, β₃adrenergic agonists, dopamine agonists (such as bromocriptine), melanocyte-stimulating hormone analogs, 5HT2c agonists, melanin concentrating hormone antagonists, leptin (the OB protein), leptin analogs, leptin agonists, galanin antagonists, lipase inhibitors (such as tetrahydrolipstatin, i.e. orlistat), anorectic agents (such as a bombesin agonist), neuropeptide-Y antagonists (e.g., NPY Y5 antagonists), PYY₃₋₃₆(including analogs thereof), thyromimetic agents, dehydroepiandrosterone or an analog thereof, glucocorticoid agonists or antagonists, orexin antagonists, glucagon-like peptide-1 agonists, ciliary neurotrophic factors (such as Axokine™ available from Regeneron Pharmaceuticals, Inc., Tarrytown, N.Y. and Procter & Gamble Company, Cincinnati, Ohio), human agouti-related protein (AGRP) inhibitors, ghrelin antagonists, histamine 3 antagonists or inverse agonists, neuromedin U agonists, MTP/ApoB inhibitors (e.g., gut-selective MTP inhibitors, such as dirlotapide), opioid antagonist, orexin antagonist, and the like.

Exemplary anti-obesity agents for use in the combination aspects of the invention include gut-selective MTP inhibitors (e.g., dirlotapide, mitratapide and implitapide, R56918 (CAS No. 403987) and CAS No. 913541-47-6), CCKa agonists (e.g., N-benzyl-2-[4-(1H-indol-3-ylmethyl)-5-oxo-1-phenyl-4,5-dihydro-2,3,6,10 b-tetraaza-benzo[e]azulen-6-yl]-N-isopropyl-acetamide described in PCT Publication No. WO 2005/116034 or US Publication No. 2005-0267100 A1), 5HT2c agonists (e.g., lorcaserin), MCR4 agonist (e.g., compounds described in U.S. Pat. No. 6,818,658), lipase inhibitor (e.g., Cetilistat), PYY₃₋₃₆(as used herein “PYY₃₋₃₆” includes analogs, such as peglated PYY₃₋₃₆ e.g., those described in US Publication 2006/0178501), opioid antagonists (e.g., naltrexone), oleoyl-estrone (CAS No. 180003-17-2), obinepitide (TM30338), pramlintide (Symlin®), tesofensine (NS2330), leptin, liraglutide, bromocriptine, orlistat, exenatide (Byetta®), AOD-9604 (CAS No. 221231-10-3) and sibutramine. Compounds of the invention and combination therapies may be administered in conjunction with exercise and a sensible diet.

Embodiments of the invention are illustrated by the following Examples. It is to be understood, however, that the embodiments of the invention are not limited to the specific details of these Examples, as other variations thereof will be known, or apparent in light of the instant disclosure, to one of ordinary skill in the art.

### Example

Unless specified otherwise, starting materials are generally available from commercial sources such as Aldrich Chemicals Co. (Milwaukee, Wis.), Lancaster Synthesis, Inc. (Windham, N.H.), Acros Organics (Fairlawn, N.J.), Maybridge Chemical Company, Ltd. (Cornwall, England), Tyger Scientific (Princeton, N.J.), and AstraZeneca Pharmaceuticals (London, England).

### General Experimental Procedures

NMR spectra were recorded on a Varian Unity™ 400 (available from Varian Inc., Palo Alto, Calif.) at room temperature at 400 MHz for proton. Chemical shifts are expressed in parts per million (δ) relative to residual solvent as an internal reference. The peak shapes are denoted as follows: s, singlet; d, doublet; dd, doublet of doublet; t, triplet; q, quartet; m, multiplet; bs, broad singlet; 2s, two singlets. Atmospheric pressure chemical ionization mass spectra (APCI) were obtained on a Fisons™ Platform II Spectrometer (carrier gas: acetonitrile: available from Micromass Ltd, Manchester, UK). Chemical ionization mass spectra (CI) were obtained on a Hewlett-Packard™ 5989 instrument (ammonia ionization, PBMS: available from Hewlett-Packard Company, Palo Alto, Calif.). Electrospray ionization mass spectra (ES) were obtained on a Waters™ ZMD instrument (carrier gas: acetonitrile: available from Waters Corp., Milford, Mass.). High resolution mass spectra (HRMS) were obtained on an Agilent™ Model 6210 using time of flight method. Where the intensity of chlorine or bromine-containing ions are described, the expected intensity ratio was observed (approximately 3:1 for ³⁵Cl/³⁷Cl-containing ions and 1:1 for ⁷⁹Br/⁸¹Br-containing ions) and the intensity of only the lower mass ion is given. In some cases only representative ¹H NMR peaks are given. Optical rotations were determined on a PerkinElmer™ 241 polarimeter (available from PerkinElmer Inc., Wellesley, Mass.) using the sodium D line (λ=589 nm) at the indicated temperature and are reported as follows [α]_{D}^{temp}, concentration (c=g/100 ml), and solvent.

Column chromatography was performed with either Baker™ silica gel (40 μm, J. T. Baker, Phillipsburg, N.J.) or Silica Gel 50 (EM Sciences™, Gibbstown, N.J.) in glass columns or in Flash 40 Biotage™ columns (ISC, Inc., Shelton, Conn.) or Biotage™ SNAP cartridge KPsil or Redisep Rf silica (from Teledyne™ Isco™) under low nitrogen pressure.

The compounds listed in Table 1 (Pharmacological Testing section) and illustrated below were prepared according to one or more of the Schemes described above.

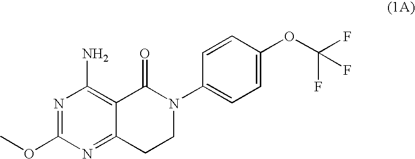

### 4-amino-2-methoxy-6-[4-(trifluoromethoxy)phenyl]-7,8-dihydropyrido[4,3-d]pyrimidin-5(6H)-one

To degassed toluene (200 mL) was added palladium acetate (0.21 g, 0.9 mmol) and x-phos (0.89 g, 1.9 mmol) and the mixture degassed a further 5 minutes. To the reaction mixture was then added cesium carbonate (24.3 g, 74.6 mmol), beta alanine ethyl ester hydrochloride (4.3 g, 28 mmol) and 1-bromo-4-trifluoromethoxybenzene (4.5 g, 18.7 mmol). The reaction mixture was heated at reflux for 6 hours, cooled and filtered through a pad of celite. Filtrate concentrated and the residue purified on silica gel eluting with a gradient from 10% to 15% ethyl acetate in heptane to give ethyl 2-(4-(trifluoromethoxy)phenylamino)acetate (3.35 g, 65%) as a yellow oil.

1H NMR (300 MHz, CDCl3): δ ppm 1.26 (t, 3H), 2.60 (t, 2H), 3.42 (t, 2H), 4.16 (q, 2H), 6.57 (dd, 2H), 7.03 (dd, 2H).

To a suspension of cyanoacetic acid (1.23 g, 15 mmol) and dimethylformamide (0.05 mL) in dichloromethane (30 mL) was added oxalyl chloride (1.23 mL, 15 mmol) and the mixture stirred for 90 minutes. To this was then added ethyl 2-(4-(trifluoromethoxy)phenylamino)acetate (3.35 g, 12 mmol) and the reaction cooled to 0° C. To the reaction was then added triethylamine (4.2 mL, 30 mmol), stirred for 1 hour and then warmed to room temperature. After being stirred for 18 hours the reaction was washed with saturated aqueous sodium bicarbonate (50 mL), dried over magnesium sulfate and evaporated. The residue (ethyl 3-(2-cyano-N-(4-(trifluoromethoxy)phenyl)-acetamido)propanoate) was shown by NMR to be pure enough to be used without further purification in the following step.

A mixture of ethyl 3-(2-cyano-N-(4-(trifluoromethoxy)phenyl)-acetamido)propanoate (4.2 g, 12.2 mmol), 1,8-diazabicycloundec-7-ene (2.2 mL, 14.6 mmol) and methanol (50 mL) was heated at reflux for 2.5 h. The reaction was then cooled, concentrated and dissolved in water (75 mL). 2M aqueous hydrochloric acid (17 mL) was added drop wise to form a brown gum. The aqueous phase was removed and the residual gum dissolved in ethyl acetate (25 mL) and 2M hydrochloric acid (25 mL). The organic phase was separated, dried over magnesium sulfate and concentrated to 10 mL. To this was added hexanes (5 mL) and the resulting yellow precipitate filtered, washed with hexanes and dried to give 4-hydroxy-2-oxo-1-(4-(trifluoromethoxy)phenyl)-1,2,5,6-tetrahydropyridine-3-carbonitrile (2.57 g, 71%).

1H NMR (300 MHz, D6-DMSO): δ ppm 2.79 (t, 2H), 3.78 (2H, t), 7.30-7.40 (m, 4H)

To a suspension of 4-hydroxy-2-oxo-1-(4-(trifluoromethoxy)phenyl)-1,2,5,6-tetrahydropyridine-3-carbonitrile (2.57 g, 8.6 mmol) and dimethylformamide (0.05 mL) in dichloromethane (30 mL) was added oxalyl chloride (2.26 mL, 26.7 mmol) and the mixture stirred for 90 minutes. The volatiles were removed and the residue co-evaporated with toluene. To the residue was added methanol (30 mL) and the mixture heated at reflux for 4 hours, cooled and evaporated. The resdiue obtained was re-crystallised from methanol to give 4-methoxy-2-oxo-1-(4-(trifluoromethoxy)phenyl)-1,2,5,6-tetrahydropyridine-3-carbonitrile (1.96 g, 68%).

1H NMR (300 MHz, D6-DMSO): δ ppm 3.04 (t, 2H), 3.86 (t, 2H), 4.02 (s, 3H), 7.42 (m, 4H)

A mixture of 4-methoxy-2-oxo-1-(4-(trifluoromethoxy)phenyl)-1,2,5,6-tetrahydropyridine-3-carbonitrile (1.91 g, 5.9 mmol), 1,8-diazabicycloundec-7-ene (1.32 mL, 8.9 mmol), O-methylisourea (2.09 g, 18.9 mmol) and methanol (75 mL) was heated at reflux for 18 hours. The reaction cooled, evaporated and the residue dissolved in ethyl acetate (50 mL). The organic solution was washed with brine (2×50 mL), dried over magnesium sulfate, filtered and concentrated. The residue was re-crystallised from ethyl acetate to give the title compound (1A) (990 mG, 47%) as a white solid.

1H NMR (300 MHz, DMSO-d6): δ ppm 2.93 (t, sH), 3.82 (s, 3H), 3.89 (t, 2H), 7.35-7.45 (m, 4H), 7.83 (db, NH), 8.28 (bd, NH).

m/z (M+1)=354.9

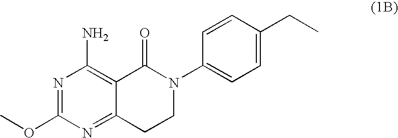

### 4-amino-6-(4-ethylphenyl)-2-methoxy-7,8-dihydropyrido[4,3-d]pyrimidin-5(6H)-one

Prepared analogous to (1A) using 1-bromo-4-ethylbenzene.

(1B): 1H NMR (400 MHz, DMSO-d6) d ppm 1.17 (t, J=7.52 Hz, 3H) 2.59 (q, J=7.48 Hz, 2H) 2.93 (t, J=6.74 Hz, 2H) 3.57-4.11 (m, 2H) 3.63-3.96 (m, 5H) 7.75 (d, 2H) 8.36 (d, J=3.71 Hz, 2H)

m/z (M+1)=299.3

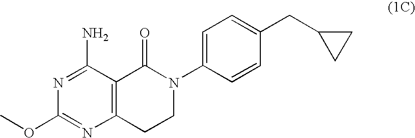

### 4-amino-6-[4-(cyclopropylmethyl)phenyl]-2-methoxy-7,8-dihydropyrido[4,3-d]pyrimidin-5(6H)-one

Prepared analogous to (1A) using 1-bromo-4-(cyclopropylmethyl)benzene which was prepared as described in Tetrahedron, 61 (42), 10138-10145, 2005.

(1C): 1H NMR (400 MHz, CHLOROFORM-d) δ ppm 0.15-0.26 (m, 2H) 0.46-0.56 (m, 2H) 0.92-1.06 (m, 1H) 2.54 (d, J=6.83 Hz, 2H) 3.04 (t, J=6.83 Hz, 2H) 3.86-3.97 (m, 5H) 5.51 (br. s., 1H) 7.17-7.23 (m, 2H) 7.30 (d, J=8.59 Hz, 2H) 8.63 (br. s., 1H)

m/z (M+1)=325.1

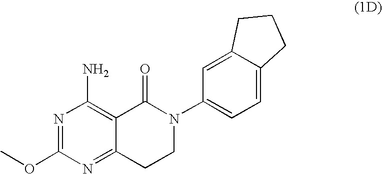

### 4-amino-6-(2,3-dihydro-1H-inden-5-yl)-2-methoxy-7,8-dihydropyrido[4,3-d]pyrimidin-5(6H)-one

Prepared analogous to bromide example using commercially available 5-bromo-2,3-dihydro-1H-indene.

(1D): 1H NMR (400 MHz, CHLOROFORM-d) δ ppm 2.06-2.14 (m, 2H) 2.92 (s, 4H) 3.05 (t, J=6.83 Hz, 2H) 3.91 (t, J=6.83 Hz, 2H) 3.96 (s, 3H) 5.50 (br. s., 1H) 7.04 (d, J=9.76 Hz, 1H) 7.16 (s, 1H) 7.25-7.28 (m, 1H) 8.65 (br. s., 1H)

m/z (M+1)=311.3

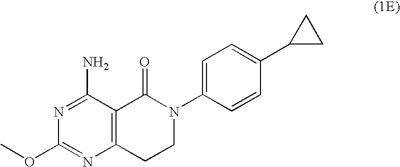

### 4-amino-6-(4-cyclopropylphenyl)-2-methoxy-7,8-dihydropyrido[4,3-d]pyrimidin-5(6H)-one

Prepared analogous to (1A) using commercially available 1-bromo-4-cyclopropylbenzene.

(1E): 1H NMR (400 MHz, CHLOROFORM-d) δ ppm 0.65-0.70 (m, 2H) 0.90-0.98 (m, 2H) 1.85-1.93 (m, 1H) 3.03 (t, J=6.73 Hz, 2H) 3.89 (t, J=6.83 Hz, 2H) 3.94 (s, 3H) 5.49 (br. s., 1H) 7.10 (s, 2H) 7.14-7.20 (m, 2H) 8.63 (br. s., 1H)

m/z (M+1)=311.3

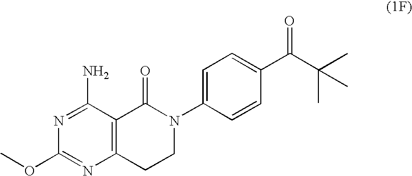

### 4-amino-6-[4-(2,2-dimethylpropanoyl)phenyl]-2-methoxy-7,8-dihydropyrido[4,3-d]pyrimidin-5(6H)-one

Prepared analogous to (1A) using commercially available 1-(4-bromophenyl)-2,2-dimethylpropan-1-one.

(1F): 1H NMR (400 MHz, MeOD): δ ppm 7.79 (2H, d), 7.44 (2H, d), 3.98 (2H, t), 3.93 (3H, s), 3.01 (2H, t) and 1.34 (9H, s).

m/z (M+1)=355.2

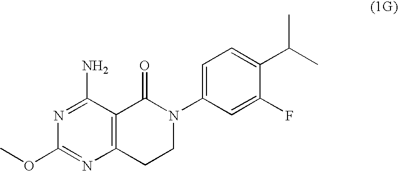

### 4-amino-6-(3-fluoro-4-isopropylphenyl)-2-methoxy-7,8-dihydropyrido[4,3-d]pyrimidin-5(6H)-one

Prepared analogous to (1A) from the 4-bromo-2-fluoro-1-isopropylbenzene which was prepared as follows:

4-Bromo-2-fluorobenzoic acid (4 g, 20 mmol) dissolved in tetrahydrofuran (281 mL) cooled to 0° C. 3M Methylmagnesium chloride in ether (27.4 mL, 82.2 mmol) was added. After addition was complete, the reaction was warmed to room temperature and stirred for 18 hours. Saturated aqueous ammonium chloride and 1N aqueous hydrochloric acid added until aqueous layer was acidic and reaction concentrated. Reaction diluted with ethyl acetate and layers separated. Organic layer washed with brine then dried over magnesium sulfate, filtered and concentrated to give 2-(4-bromo-2-fluorophenyl)propan-2-ol (3.64 g, 90%) as a yellowish solid.

1H NMR (400 MHz, CHLOROFORM-d) δ ppm 1.60 (s, 6H) 6.94-7.34 (m, 2H) 7.45 (t, J=8.72 Hz, 1H)

2-(4-Bromo-2-fluorophenyl)propan-2-ol (3.64 g, 15.6 mmol) dissolved in dichloromethane (156 ml) at room temperature. Triethylsilane (3.74 ml, 23.4 mmol) was added followed by trifluoroacetic acid (12 ml, 156 mmol) and the resulting solution was stirred at room temperature for 2 hours. Reaction concentrated and purified on silica gel eluting with heptane to give 4-bromo-2-fluoro-1-isopropylbenzene (0.18 g, 33%) as a clear oil.

1H NMR (400 MHz, CHLOROFORM-d) δ ppm 1.22 (d, J=7.06 Hz, 6H) 3.10-3.22 (m, 1H) 7.06-7.24 (m, 3H)

(1G): 1H NMR (500 MHz, CHLOROFORM-d) δ ppm 1.28 (d, 6H) 3.07 (t, 2H) 3.23-3.27 (m, 1H) 3.94 (t, 2H) 3.98 (s, 3H) 5.58 (s, 1H) 7.01-7.03 (m, 1H) 7.06-7.08 (m, 1H) 7.29-7.31 (m, 1H) 8.61 (s, 1H)

m/z (M+1)=331.5

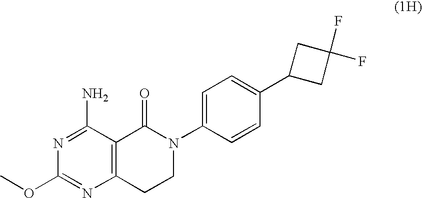

### 4-amino-6-[4-(3,3-difluorocyclobutyl)phenyl]-2-methoxy-7,8-dihydropyrido[4,3-d]pyrimidin-5(6H)-one

Prepared analogous to (1A) from 1-bromo-4-(3,3-difluorocyclobutyl)benzene which was prepared as follows:

3-(4-Bromophenyl)cyclobutanone (600 mg, 2.67 mmol) was dissolved in dichloromethane (10 mL) and toluene (10 mL). Boron trifluoride diethyl etherate (0.676 mL, 5.33 mmol) was added and reaction cooled to 0° C. Deoxo-Fluor® (0.983 mL, 5.33 mmol) was added drop wise and once addition was complete, the reaction was warmed to room temperature for 48 hours. 1M aqueous sodium hydroxide (10 ml) was added and vigorously stirred for 30 minutes. Reaction was extracted with dichloromethane (50 mL), dried over sodium sulfate, filtered and concentrated. Crude purified on silica gel, eluting with a gradient from 0% to 8% ethyl acetate in heptane to give 1-bromo-4-(3,3-difluorocyclobutyl)benzene (360 mg, 54%) as a colorless oil.

1H NMR (400 MHz, CHLOROFORM-d) δ ppm 2.53-2.72 (m, 2H) 2.92-3.07 (m, 2H) 3.26-3.40 (m, 1H) 7.06-7.13 (m, 2H) 7.41-7.49 (m, 2H)

(1H): 1H NMR (400 MHz, METHANOL-d4) δ ppm 2.54-2.72 (m, 2H) 2.88-3.05 (m, 4H) 3.31-3.44 (m, 1H) 3.84-3.95 (m, 5H) 7.14-7.36 (m, 4H)

m/z (M+1)=361.1

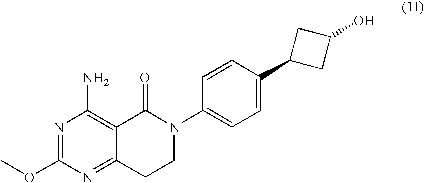

### 4-amino-6-[4-(trans-3-hydroxycyclobutyl)phenyl]-2-methoxy-7,8-dihydropyrido[4,3-d]pyrimidin-5(6H)-one

Prepared analogous to (1A) using commercially available (1s,3s)-3-(4-bromophenyl)cyclobutanol.

(1I): 1H NMR (400 MHz, CHLOROFORM-d) δ ppm 2.39-2.54 (m, 4H) 3.06 (t, J=6.83 Hz, 2H) 3.61-3.69 (m, 1H) 3.93 (t, J=6.83 Hz, 2H) 3.96 (s, 3H) 4.51-4.59 (m, 1H) 5.53 (br. s., 1H) 7.22-7.31 (m, 4H) 8.63 (br. s., 1 H

m/z (M+1)=341.3

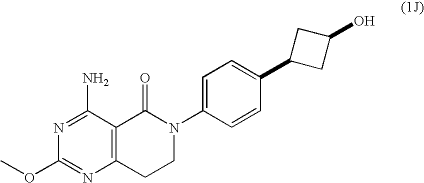

### 4-amino-6-[4-(cis-3-hydroxycyclobutyl)phenyl]-2-methoxy-7,8-dihydropyrido[4,3-d]pyrimidin-5(6H)-one

Prepared analogous to (1A) using commercially available (1r,3r)-3-(4-bromophenyl)cyclobutanol.

(1J): 1H NMR (400 MHz, CHLOROFORM-d) δ ppm 1.96-2.05 (m, 2H) 2.72-2.80 (m, 2H) 2.90-3.00 (m, 1H) 3.04 (t, J=6.83 Hz, 2H) 3.91 (t, J=6.83 Hz, 2H) 3.94 (s, 3H) 4.23-4.33 (m, 1H) 5.51 (br. s., 1H) 7.22 (s, 2H) 7.26 (s, 2H) 8.61 (br. s., 1H)

m/z (M+1)=341.3

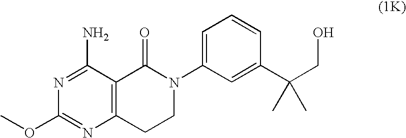

### 4-amino-6-[3-(2-hydroxy-1,1-dimethylethyl)phenyl]-2-methoxy-7,8-dihydropyrido[4,3-d]pyrimidin-5(6H)-one

Prepared analogous to (1A) from (2-(4-bromophenyl)-2-methylpropoxy)(tert-butyl)dimethylsilane which was prepared as follows:

3-Bromophenylacetic acid (100 g, 0.47 mol) was dissolved in methanol (1000 ml) and concentrated sulfuric acid (1 ml) added and the mixture heated at reflux overnight. The methanol was evaporated and the residue partitioned between dichloromethane (600 ml) and saturated aqueous sodium bicarbonate (200 ml). The organic layer was washed with brine (300 ml), dried over magnesium sulfate and concentrated to give methyl 2-(3-bromophenyl)acetate (102 g, 0.45 mol, 95%) as an oil.

1H NMR (400 MHz, CDCl3): δ ppm 7.43 (bt, 1H), 7.38 (dt, 1H), 7.15-7.19 (m, 2H), 3.68 (s, 3H), 3.58 (s, 2H)

Sodium hydride (60% in oil) (10.4 g, 436 mmol) was added to tetrahydrofuran (400 ml) under argon and heated with stirring to 50° C. Methyl 2-(3-bromophenyl)acetate (20 g, 87.3 mmol) was added drop wise over 30 minutes and heating continued for 90 minutes. The temperature was lowered to below 40° C. and methyl iodide (13 ml, 209 mmol) was added over 10 minutes. The resulting suspension was stirred at room temperature overnight. Water (300 ml) was carefully added and reaction mixture concentrated. Residue was partitioned between diethyl ether (400 ml) and water. The aqueous layer was extracted with diethyl ether (400 ml), and the combined ethereal extracts were dried over sodium sulfate and concentrated. Crude oil was purified by column chromatography, eluting with a gradient from 0% to 20% ethyl acetate in heptane to give methyl 2-(3-bromophenyl)-2-methylpropanoate (14.96 g, 58 mmol, 67%).

1H NMR (400 MHz, CDCl3): δ ppm 7.47 (t, 1H), 7.37 (dt, 1H), 7.24 (dt, 1H), 7.18 (t, 1H), 3.65 (s, 3H), 1.55 (s, 6H)

Lithium aluminium hydride (2.12 g, 55.9 mmol) was dissolved in tetrahydrofuran (400 ml) and cooled to 0° C. Methyl 2-(3-bromophenyl)-2-methylpropanoate (19.17 g, 74.5 mmol) was dissolved in tetrahydrofuran (100 ml) and added drop wise to the hydride solution, keeping the internal temperature below 8° C. This was stirred for 3 hours and then quenched by the cautious drop wise addition of water (2.12 ml), 15% aqueous sodium hydroxide solution (2.12 ml) and water (6.36 ml). The resulting suspension was stirred for 1 hour and then filtered, washing the solid with ethyl acetate. The resulting organic solution was dried over magnesium sulfate, filtered and evaporated to give 2-(3-bromophenyl)-2-methylpropan-1-ol (16.91 g, 73.8 mmol, 99%, contains 5% unreacted ester).

1H NMR (400 MHz, CDCl3) δ ppm 7.51 (t, 1H), 7.35 (dt, 1H), 7.31 (dt, 1H), 7.20 (t, 1H), 3.59 (s, 2H), 1.35 (b s, 1H), 1.31 (s, 6H)

2-(3-Bromophenyl)-2-methylpropan-1-ol (16.91 g, 73.8 mmol) was dissolved in dimethylformamide (100 ml) and cooled to 0° C. Imidazole (10.04 g, 147.6 mmol) and tert-butyldimethylsilyl chloride (13.34 g, 88.5 mmol) were added. The solution was allowed to warm to room temperature with stirring for 1 hour before the solvent was evaporated. Water (200 ml) was added, and the product extracted with ethyl acetate (3×200 ml). The organics were washed with 10% aqueous citric acid (100 ml), water (2×50 ml) and saturated aqueous sodium bicarbonate (50 ml). The solvent was removed and the residue dissolved in diethyl ether (200 ml) and washed with water (2×50 ml), brine (50 ml),dried over magnesium sulfate and evaporated to give (2-(3-bromophenyl)-2-methylpropoxy)(tert-butyl)dimethylsilane (24.72 g, 71.99 mmol, 97%).

1H NMR (400 MHz, CDCl3) δ ppm 7.51 (t, 1H), 7.29 (m, 2H), 7.15 (t, 1H), 3.49 (s, 2H), 1.27 (s, 6H), 0.83 (s, 9H), −0.07 (s, 6H)

LCMS [M+H]+=321.4, 95.11%

(1K): 1H NMR (400 MHz, DMSO) δ ppm 8.35 (bd, 1H), 7.75 (bd, 1H), 7.25-7.30 (m, 2H), 7.21 (bdt, 1H), 7.11 (bdt, 1H), 4.67 (t, 1H), 3.85 (t, 2H), 3.82 (s, 3H), 3.38 (d, 2H), 2.92 (t, 2H), 1.19 (s, 6H)

m/z (M+1)=343.4

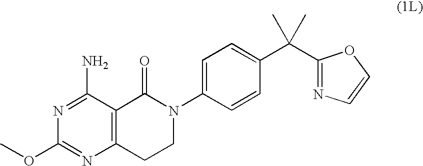

### 4-amino-2-methoxy-6-{4-[1-methyl-1-(1,3-oxazol-2-yl)ethyl]phenyl}-7,8-dihydropyrido[4,3-d]pyrimidin-5(6H)-one

Prepared analogous to (1A) from 2-(2-(4-bromophenyl)propan-2-yl)oxazole which was prepared as follows:

Methyl 2-(4-bromophenyl)-2-methylpropanoate (24 g, 93.3 mmol), 2 M aqueous lithium hydroxide (200 mL) and 1,4-dioxane (250 mL) were heated to 50° C. for 5.5 hours. The reaction mixture was cooled to room temperature, made acidic with 2 M (aqueous hydrochloric acid and diluted with brine. The reaction mixture was extracted with ethyl acetate, dried over magnesium sulfate and concentrated to give 2-(4-bromophenyl)-2-methylpropanoic acid (21.4 g, 94%).

1H NMR (300 MHz, CDCl3): δ ppm 7.46 (dd, 2H), 7.27 (dd, 2H), 1.55 (s, 6H) ppm.

To a solution of 2-(4-bromophenyl)-2-methylpropanoic acid (9.9 g, 40.7 mmol) and dimethylformamide (0.1 g) in dichloromethane (150 mL) was added oxalyl chloride (4.5 mL, 53 mmol) at room temperature for 2 hours. Reaction concentrated and the residue dissolved in sulfolane (125 mL). 1,2,3-Triazole (3.1 g, 44.8 mmol) and potassium carbonate (11.8 g, 85.6 mmol) were added and the mixture heated 120° C. for 1 hour. The reaction was cooled, diluted with ethyl acetate and washed with 1:1 brine/water (6×600 mL). The organic phase was dried over magnesium sulfate and concentrated. Crude was purified by chromatography on silica eluting with 25% ethyl acetate in hexanes to give 2-(2-(4-bromophenyl)propan-2-yl)oxazole (4.9 g, 43%).

1H NMR (300 MHz, CDCl3): δ ppm 7.55 (s, 1H), 7.41 (dd, 2H), 7.11 (dd, 2H), 1.75 (s, 6H) ppm.

(1L): 1H NMR (400 MHz, CDCl3): δ ppm 7.55 (d, J=0.9 Hz, 1H), 7.31-7.24 (m, 4H), 7.06 (d, J=0.9 Hz, 1H), 3.95 (s, 3H), 3.92 (t, J=6.9 Hz, 2H), 3.03 (t, J=6.9 Hz, 2H), 1.79 (s, 6H) ppm.

m/z (M+1)=380.0

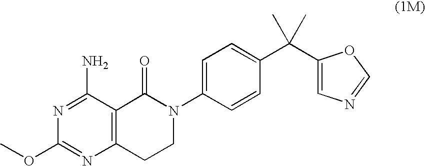

### 4-amino-2-methoxy-6-{4-[1-methyl-1-(1,3-oxazol-5-yl)ethyl]phenyl}-7,8-dihydropyrido[4,3-d]pyrimidin-5(6H)-one

Prepared analogous to (1A) from 5-(2-(4-bromophenyl)propan-2-yl)oxazole which was prepared as follows:

Sodium hydride (12 g, 0.3 mmol) was added to tetrahydrofuran (300 mL) at room temperature. Bromophenyl acetonitrile (20 g, 0.1 mol) in tetrahydrofuran (100 mL) was added drop wise to the reaction solution over a period of two hours. Methyl iodide (15 mL, 0.24 mol) in tetrahydrofuran (100 mL) was added drop wise keeping the internal temperature between 24 -30° C. The solution was then stirred for two days at room temperature. Cold water (300 mL) was then added drop wise to the suspension over a period of one hour. Ethyl acetate (250 mL) was added and the layers separated. The aqueous layer was extracted with ethyl acetate (200 mL), dried over magnesium sulfate and concentrated. The crude product was purified via column chromatography on silica gel eluting with 3% ethyl acetate in hexanes to give 3-(4-bromophenyl)-3-methylbutanenitrile (22 g, 98% yield) as a yellow oil.

1H NMR (300 MHz CDCl3) δ ppm 7.50 (d, 2H), 7.35 (d, 2H), 1.72 (s, 6H).

Diisobutylaluminum hydride (1M in dichloromethane, 107 mL, 160.5 mmol) was added drop wise to a stirred solution of 2-(4-bromophenyl)-2-methylpropanal (22 g, 97.8 mmol) in tetrahydrofuran (250 mL) at −20° C. The solution was stirred for two hours −20° C., then slowly warmed to room temperature over night. Reaction cooled to 0° C. and ice water (250 mL) was slowly added. After addition was complete, aqueous 1 M hydrochloric acid (200 mL) and ethyl acetate (200 mL) were added and the layers separated. The organic layer was washed with aqueous 1 M hydrochloric acid (200 mL), dried over magnesium sulfate and concentrated. The crude product was purified via column chromatography on silica gel eluting with 1% ethyl acetate in hexanes to give 2-(4-bromophenyl)-2-methylpropanal (16 g, 73%) as a yellow oil.

1H NMR (400 MHz, CDCl3) δ ppm 9.46 (s, 1H), 7.51 (d, 2H), 7.14 (d, 2H), 1.44 (s, 6H).

A suspension of 2-(4-bromophenyl)-2-methylpropanal (16 g, 71 mmol) and poptassium carbonate (23.7 g, 171 mmol) in methanol (200 mL) was stirred for five minutes. Tosylmethyl isocyanide (12.6 g, 71 mmol) was added portion wise to reaction suspension and then heated to reflux overnight. The solution was cooled to room temperature and solids filtered off. Filtrate concentrated. Water (100 mL) and ethyl acetate (200 mL) were added and the layers separated. The organic layer was washed with water (100 mL) and dried over magnesium sulfate and concentrated. The crude product was purified via column chromatography eluting with 10% ethyl acetate in hexanes to give 5-(2-(4-bromophenyl)propan-2-yl)oxazole (8.6 g, 46%) as a yellow oil.

1H NMR (400 MHz CDCl3) δ ppm 7.76 (s, 1H), 7.41 (d, 2H), 7.11 (d, 2H), 6.85 (s, 1H), 1.66 (s, 6H).

(1M): 1H NMR (CDCl3, 400 MHz) δ ppm 8.60 (s, 1H), 7.77 (s, 1H), 7.26 (m, 6H), 6.88 (s, 1H), 5.51 (s, 1H), 3.92 (m, 5H), 3.05 (m, 2H), 1.67 (s, 3H).

m/z (M+1)=380.5

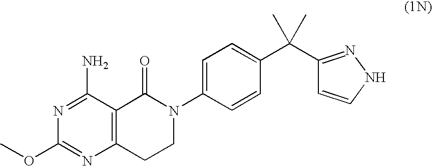

### 4-amino-2-methoxy-6-{4-[1-methyl-1-(1H-pyrazol-3-yl)ethyl]phenyl}-7,8-dihydropyrido[4,3-d]pyrimidin-5(6H)-one

Prepared analogous to (1A) from 1-(4-methoxybenzyl)-3-(2-(4-bromophenyl)propan-2-yl)-1H-pyrazole which was prepared as follows:

Methyl 2-(4-bromophenyl)-2-methylpropanoate (43.0 g, 0.17 mol) was dissolved in tetrahydrofuran (450 mL) and N,O-dimethylhydroxylamine (24.5 g, 0.25 mol) was added. The mixture was cooled to -20° C. and iso-propyl magnesium chloride (250 mL, 0.50 mol) was added drop wise. After the addition was complete, the mixture was warmed to room temperature and stirred for 1.5 hours and then heated to 30° C. for 1 hour. The mixture was then cooled to 0° C. and saturated aqueous ammonium chloride (200 mL) was added. The mixture was separated and the aqueous layer was washed with ethyl acetate (500 mL). The organic layers were combined, dried over magnesium sulfate and concentrated. Crude purified by flash column chromatography to give 2-(4-bromophenyl)-N-methoxy-N,2-dimethylpropanamide (36.7 g, 77%) as a yellow oil.

1H NMR (CDCl3, 400 MHz): 7.42 (d, 2H), 7.12 (d, 2H), 3.25 (s, 3H), 2.70 (s, 3H), 1.55 (s, 6H).

2-(4-Bromophenyl)-N-methoxy-N,2-dimethylpropanamide (100.0 g, 0.35 mol) was dissolved in tetrahydrofuran (1 L) and the mixture was cooled to −20° C. Methylmagnesium bromide (3M, 174 mL) was added drop wise and the mixture was allowed to warm to room temperature and stirred for 16 hours. Reaction not complete; additional 0.25 eq of methylmagnesium bromide was added and the mixture was heated to 40° C. for 1 hour. The mixture was then cooled to 0° C. and water (500 mL) then 1 M aqueous hydrochloric acid (1 L) were added. The mixture was separated and the aqueous layer was washed with ethyl acetate (500 mL). The organic layers were combined, washed with brine, dried over magnesium sulfate and concentrated to give 3-(4-bromophenyl)-3-methylbutan-2-one (84.2 g, 100%) as a clear oil.

1H NMR (CDCl3, 400 MHz): δ ppm 7.42 (d, 2H), 7.10 (d, 2H), 1.89 (s, 3H), 1.42 (s, 6H).

3-(4-Bromophenyl)-3-methylbutan-2-one (52.3 g, 0.22 mol) was dissolved in dimethylformamide-dimethylacetal (51.6 g, 0.43 mol) and the mixture was heated to reflux for 48 hours. The mixture was cooled to room temperature and concentrated. The crude product was purified by flash column chromatography to give (E)-4-(4-bromophenyl)-1-(dimethylamino)-4-methylpent-1-en-3-one (24.7 g, 39%) as a yellow solid.

1H NMR (CDC3, 400 MHz): δ ppm 7.52 (d, 1H), 7.49 (d, 2H), 7.13 (d, 2H), 4.69 (d, 1H), 2.90-3.10 (bs, 3H), 2.50-2.70 (bs, 3H), 1.44 (s, 6H).

(E)-4-(4-bromophenyl)-1-(dimethylamino)-4-methylpent-1-en-3-one (28.0 g, 0.095 mol) was dissolved in ethanol (300 mL) and hydrazine monohydrate (5.2 g, 0.104 mol) was added. The mixture was heated to reflux for 6 hours then cooled to room temperature. Reaction concentrated to give 3-(2-(4-bromophenyl)propan-2-yl)-1H-pyrazole (22.4 g, 90%) as a yellow oil.

1H NMR (CDCl3, 400 MHz): δ ppm 7.36 (d, 2H), 7.36 (s, 1H), 7.10 (d, 2H), 6.07 (s, 1H), 1.66

3-(2-(4-Bromophenyl)propan-2-yl)-1H-pyrazole (22.4 g, 0.084 mol) was dissolved in acetone (200 mL) and 4-methoxybenzyl chloride (13.6 g, 0.087 mol) and potassium carbonate (30.4 g, 0.22 mol) were added. The mixture was heated to reflux for 16 hours then cooled to room temperature and concentrated. Crude product purified by flash column chromatography to give 1-(4-methoxybenzyl)-3-(2-(4-bromophenyl)propan-2-yl)-1H-pyrazole (20.1 g, 62%).

Prepared analogous to bromide example from 1-(4-methoxybenzyl)-3-(2-(4-bromophenyl)propan-2-yl)-1H-pyrazole. After formation of the pyrimidine ring, de-protection of the pyrazole ring gave the desired product:

6-(4-(2-(1-(4-methoxybenzyl)-1H-pyrazol-3-yl)propan-2-yl)phenyl)-4-amino-2-methoxy-7,8-dihydropyrido[4,3-d]pyrimidin-5(6H)-one (0.8 g, 1.6 mmol) was dissolved in ethyl acetate (400 mL) and palladium on carbon (5%) was added. The mixture was stirred at 80° C. for 5 hours at 40 bar of hydrogen then at room temperature for 64 hours and then at 80° C. at 40 bar hydrogen for 7 hours then at room temperature overnight. The mixture was filtered through a pad of celite and washed with hot ethyl acetate and the filtrate concentrated. The crude product was purified by flash column chromatography to give 4-amino-2-methoxy-6-{4-[1-methyl-1-(1H-pyrazol-3-yl)ethyl]phenyl}-7,8-dihydropyrido[4,3-d]pyrimidin-5(6H)-one (38.3 mg, 6%).

1H NMR (DMSO, 400 MHz): δ ppm 7.48 (bs, 1H), 7.31 (d, 2H), 7.23 (d, 2H), 6.14 (bs, 1H), 3.92 (s, 3H), 3.90 (t, 2H), 2.94 (t, 2H), 1.70 (s, 6H).

(1N): 1H NMR (DMSO, 400 MHz): δ ppm 7.48 (bs, 1H), 7.31 (d, 2H), 7.23 (d, 2H), 6.14 (bs, 1H), 3.92 (s, 3H), 3.90 (t, 2H), 2.94 (t, 2H), 1.70 (s, 6H).

m/z (M+1)=379.1

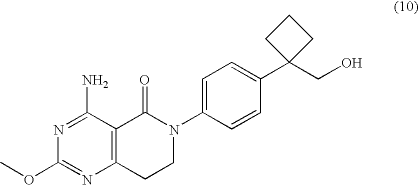

### 4-amino-6-{4-[1-(hydroxymethyl)cyclobutyl]phenyl}-2-methoxy-7,8-dihydropyrido[4,3-d]pyrimidin-5(6H)-one

Prepared analogous to (1A) from ((1-(4-bromophenyl)cyclobutyl)methoxy)(tert-butyl)dimethylsilane which was prepared as follows:

4-Bromophenylacetic acid (4.9 g, 22.79 mmol) in methanol (15 mL) and sulfuric acid (0.05 mL, 0.9 mmol) was heated to 75° C. for 2 hours. Reaction concentrated and saturated aqueous sodium bicarbonate (40 mL) added and reaction mixture extracted with ethyl acetate. Organics washed with brine, dried over magnesium sulfate, filtered and concentrated to give methyl 2-(4-bromophenyl)acetate (4.99 g, 95%).

60% Sodium hydride in mineral oil (4.36 g, 109 mmol) was carefully added portion wise to a solution of the methyl 2-(4-bromophenyl)acetate (4.99 g, 21.78 mmol) in tetrahydrofuran (50 mL) and dimethylformamide (30 mL) at room temperature. Once addition was complete, reaction was stirred at room temperature for 20 minutes and then cooled to 5° C. 1,3-dibromopropane (6.160 g, 30.5 mmol) was added portion wise over 40 minutes. Once addition was complete, the reaction was stirred at room temperature for 3 hours. Acetic acid (8 mL) was slowly added to the reaction followed by water (150 mL). Solution was extracted with 1:1 ethyl acetate: heptane, dried over sodium sulfate, filtered and concentrated. Crude purified on silica gel, eluting with a gradient from 20% to 100% ethyl acetate in heptane to give 1-(4-bromophenyl)cyclobutanecarboxylic acid (1.180 g, 21%).

1H NMR (500 MHz, CHLOROFORM-d) δ ppm 1.85-1.96 (m, 1H) 2.06-2.17 (m, 1H) 2.47-2.56 (m, 2H) 2.82-2.91 (m, 2H) 7.21 (d, 2H) 7.48 (d, 2H) 11.54 (br. s., 1H) 1-(4-Bromophenyl)cyclobutanecarboxylic acid (290 mg, 1.14 mmol) in tetrahydrofuran (6 mL) was added a 1M solution of borane-tetrahydrofuran complex in tetrahydrofuran (0.231 mL, 2.27 mmol) drop wise at room temperature and stirred for 24 hours. Methanol (1 mL) was slowly added to the reaction mixture and then concentrated. Water (10 mL) and 1M aqueous hydrochloric acid added and reaction extracted with a 1:1 ethyl acetate: heptane solution. Extract washed with brine, dried over magnesium sulfate, filtered and concentrated. Crude purified on silica gel, eluting with a gradient from 20% to 80% ethyl acetate in heptane to give (1-(4-bromophenyl)cyclobutyl)methanol (265 mg, 96%).

1H NMR (400 MHz, CHLOROFORM-d) δ ppm 1.81-1.92 (m, 1H) 2.01-2.11 (m, 1H) 2.16-2.31 (m, 4H) 3.71 (s, 2H) 7.00 (d, 2H) 7.43 (d, 2H)

(1-(4-Bromophenyl)cyclobutyl)methanol (260 mg, 1.08 mmol), tert-butyldimethyichiorosilane (0.246 mL, 1.29 mmol) and imidazole (151 mg, 2.16 mmol) combined in dimethylformamide (6 mL) at room temperature for 18 hours. Water (20 mL) added and solution extracted with a 1:1 solution of ethyl acetate-heptane. The extract was washed with brine, dried over magnesium sulfate, filtered and concentrated. Crude purified on silica gel, eluting with a gradient from 0 to 30% ethyl acetate in heptane to give ((1-(4-bromophenyl)cyclobutyl)methoxy)(tert-butyl)dimethylsilane (323 mg, 84%)

1H NMR (400 MHz, CHLOROFORM-d) δ ppm −0.15 (s, 6H) 0.82 (s, 9H) 1.77-1.86 (m, 1H) 1.97-2.08 (m, 1H) 2.20-2.26 (m, 4H) 3.59 (s, 2H) 6.98 (d, 2H) 7.37 (d, 2H)

(1O): 1H NMR (500 MHz, CHLOROFORM-d) δ ppm 1.03 (d, 6H) 2.06-2.14 (m, 1H) 3.06 (t, 2H) 3.73 (d, 2H) 3.90 (t, 2H) 3.96 (s, 3H) 5.63 (s, 1H) 6.95 (d, 2H) 7.21 (d, 2H) 8.67 (s, 1H)

m/z (M+1)=355.2

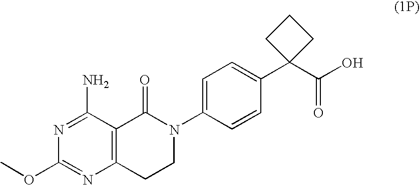

### 1-[4-(4-amino-2-methoxy-5-oxo-7,8-dihydropyrido[4,3-d]pyrimidin-6(5H)-yl)phenyl]cyclobutanecarboxylic acid

Prepared analogous to (1A) using 1-(4-bromophenyl)cyclobutanecarboxylic acid which was prepared as follows:

1-(4-Bromophenyl)cyclobutanecarboxylic acid (970 mg, 3.8 mmol), benzyl bromide (0.543 mL, 4.56 mmol) and cesium carbonate (1.73 g, 5.32 mmol) combined in dimethylformamide (9 mL) and stirred at room temperature for 18 hours and then heated to 60° C. for 24 hours. Reaction diluted with water and extracted with 1:1 ethyl acetate:

heptane. Organic layers washed with brine, dried over magnesium sulfate, filtered and concentrated. Toluene added and concentrated to give benzyl 1-(4-bromophenyl)cyclobutanecarboxylate (1.3 g, 99%) which was used in the Buchwald reaction without further purification.

1H NMR (500 MHz, CHLOROFORM-d) δ ppm 1.85-1.93 (m, 1H) 2.02-2.12 (m, 1H) 2.46-2.53 (m, 2H) 2.83-2.90 (m, 2H) 5.10 (s, 2H) 7.17-7.21 (m, 4H) 7.30-7.34 (m, 3H) 7.46 (d, 2H)

1-[4-(4-amino-2-methoxy-5-oxo-7,8-dihydropyrido[4,3-d]pyrimidin-6(5H)-yl)phenyl]cyclobutanecarboxylic acid was isolated via the de-protection of the acid detailed below:

To benzyl 1-(4-(4-amino-2-methoxy-5-oxo-7,8-dihydropyrido[4,3-d]pyrimidin-6(5H)-yl)phenyl)cyclobutanecarboxylate (70 mg, 0.15 mmol) in methanol (30 mL) was added 10% palladium on activated carbon (50 mg) and 2M aqueous solution of potassium hydroxide (0.09 mL) and allowed to stir in a parr shaker at room temperature under hydrogen (45 PSI) for 2 hours. Reaction mixture was carefully filtered through a pad of celite under a steady stream of nitrogen washing with copious amounts of ethyl acetate to provide 1-[4-(4-amino-2-methoxy-5-oxo-7,8-dihydropyrido[4,3-d]pyrimidin-6(5H)-yl)phenyl]cyclobutanecarboxylic acid (60 mg, 96%) as the potassium salt.

1H NMR (400 MHz, DMSO-d6) δ ppm 1.52-1.63 (m, 1H) 1.69-1.80 (m, 1H) 2.04-2.15 (m, 2H) 2.58-2.68 (m, 2H) 2.91 (t, 2H) 3.77-3.86 (m, 5H) 7.10 (d, 2H) 7.19 (d, 2H) 7.72 (d, 1H) 8.37 (d, 1H)

(1P): 1H NMR (400 MHz, DMSO-d6) δ ppm 1.52-1.63 (m, 1H) 1.69-1.80 (m, 1H) 2.04-2.15 (m, 2H) 2.58-2.68 (m, 2H) 2.91 (t, 2H) 3.77-3.86 (m, 5H) 7.10 (d, 2H) 7.19 (d, 2H) 7.72 (d, 1H) 8.37 (d, 1H)

m/z (M+1)=369.1

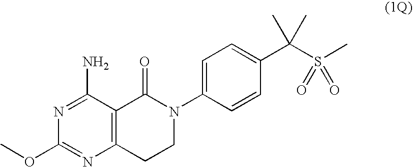

### 4-amino-2-methoxy-6-{4-[1-methyl-1-(methylsulfonyl)ethyl]phenyl}-7,8-dihydropyrido[4,3-d]pyrimidin-5(6H)-one

Prepared analogous to (1A) from 1-bromo-4-(2-(methylsulfonyl)propan-2-yl)benzene which was prepared as follows:

4-Bromobenzyl bromide (2.1 g, 8.402 mmol) and sodium methanesulfinate (2.34 g, 19 mmol) dissolved in dimethylformamide (15 mL) and heated to 60° C. for 2 hours. Reaction cooled to room temperature and water (60 mL) added and reaction mixture extracted with ethyl acetate. Organic washed with brine, dried over magnesium sulfate, filtered and concentrated to give 1-bromo-4-(methylsulfonylmethyl)benzene (1.77 g, 84%) as white solid.

1H NMR (400 MHz, CHLOROFORM-d) δ ppm 2.76 (s, 3H) 4.18 (s, 2H) 7.28 (d, 2H) 7.54 (d, 2H)

1-Bromo-4-(methylsulfonylmethyl)benzene (1.76 g, 7.065 mmol) dissolved in tetrahydrofuran (16 mL) and cooled to 0° C. A 1 M solution of potassium tert-butoxide in tetrahydrofuran (15.5 mL) was added drop wise and stirred at 0° C. for 20 minutes. Methyl iodide (968 mL, 15.5 mmol) was added drop wise at 0° C. and stirred for 30 minutes. Water (30 mL), ethyl acetate (30 mL) and heptane (30 mL) added. Organic was separated, washed with brine, dried over magnesium sulfate, filtered and concentrated to give 1-bromo-4-(2-(methylsulfonyl)propan-2-yl)benzene (1.91 g, 97%).

(1Q): 1H NMR (500 MHz, CHLOROFORM-d) δ ppm 1.88 (s, 6H) 2.61 (s, 3H) 3.11 (t, 2H) 3.99 (t, 2H) 3.99 (s, 3H) 5.63 (s, 1H) 7.40 (d, 2H) 7.71 (d, 2H) 8.62 (s, 1H)

m/z(M+1)=391

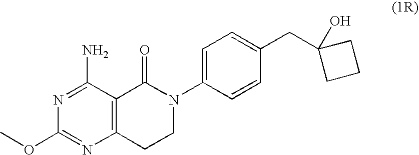

### 4-amino-6-{4-[(1-hydroxycyclobutyl)methyl]phenyl}-2-methoxy-7,8-dihydropyrido[4,3-d]pyrimidin-5(6H)-one

Prepared analogous to (1A) from 1-(4-bromobenzyl)cyclobutanol which was prepared as follows:

Magnesium turnings (4.22 g, 174 mmol) were stirred in diethyl ether (40 ml) under argon, and dibromoethane (2 drops) added. 4-Bromobenzyl bromide (21.77 g, 87 mmol) was dissolved in diethyl ether (150 ml) and a portion added to the magnesium, which was warmed until initiation of the Grignard reaction was seen. Then the remainder of the benzyl bromide solution was added at a rate to maintain gentle reflux, and then stirred for a further 30 minutes. This was then added by cannula to a solution of cyclobutanone (6.1 g, 87 mmol) in diethyl ether (100 ml) at 5° C. This was allowed to warm to room temperature over 2 hours before being quenched by the addition of saturated aqueous ammonium chloride solution (100 ml). The reaction mixture was diluted by the addition of ethyl acetate (100 ml), and the layers separated. The organic layer was washed with water (100 ml) and brine (50 ml), dried over magnesium sulfate and concentrated. The product was purified by dry flash column chromatography, eluting with 10-25% ethyl acetate in heptane to give 1-(4-bromobenzyl)cyclobutanol (4.18 g, 17 mmol, 20%)

1H NMR (400 MHz, CDCl3): δ ppm 7.43 (d, 2H), 7.13 (d, 2H), 2.85 (s, 2H), 2.10-2.13 (m, 2H), 1.92-2.03 (m, 2H), 1.66 (bs, 1H), 1.52-1.64 (m, 2H).

(1R): 1H NMR (400 MHz, MeOD) δ ppm 7.31-2.35 (m, 2H), 7.22-7.25 (m, 2H), 3.88-3.94 (m, 5H), 2.99 (t, 2H), 2.87 (s, 2H), 2.08-2.16 (m, 2H), 1.93-2.02 (m, 2H), 1.69-1.79 (m, 1H), 1.50-1.61 (m, 1H)

m/z (M+1)=355.2

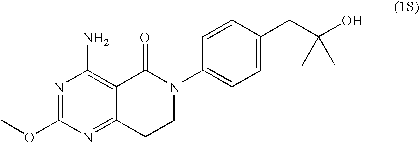

### 4-amino-6-[4-(2-hydroxy-2-methylpropyl)phenyl]-2-methoxy-7,8-dihydropyrido[4,3-d]pyrimidin-5(6H)-one

Prepared from methyl 2-(4-(4-amino-2-methoxy-5-oxo-7,8-dihydropyrido[4,3-d]pyrimidin-6(5H)-yl)phenyl)acetate which was prepared analogous to (1A) from methyl 2-(4-bromophenyl)acetate. The final step afforded the desired target as follows:

Methyl 2-(4-(4-amino-2-methoxy-5-oxo-7,8-dihydropyrido[4,3-d]pyrimidin-6(5H)-yl)phenyl)acetate (274 mg, 0.8 mmol) was dissolved in tetrahydrofuran (25 mL) under argon and cooled to 0° C. Methyl magnesium bromide (2M in diethyl ether, 1.2 mL, 2.4 mmol) was then added drop wise and the reaction mixture warmed to room temperature and stirred for 4 hours. Reaction cooled to 0° C. and saturated aqueous ammonium chloride (20 mL) added, The product was then extracted with ethyl acetate (2×50 mL), dried over magnesium sulfate, filtered and concentrated. The crude product was purified by flash chromatography eluting with 10% methanol in ethyl acetate to give 4-amino-6-[4-(2-hydroxy-2-methylpropyl)phenyl]-2-methoxy-7,8-dihydropyrido[4,3-d]pyrimidin-5(6H)-one (77 mg, 28% yield, 92% purity by LCMS).

(1S): 1H NMR (400 MHz, CDCl3): δ ppm 8.62 (br. s, 1H), 7.27-7.25 (m, 4H), 5.52 (br. s, 1H), 3.95 (s, 3H), 3.94 (t, J=6.9 Hz, 2H), 3.05 (t, J=3.9 Hz, 2H), 2.78 (s, 2H), 1.25 (s, 6H) ppm.

m/z (M+1)=343.0

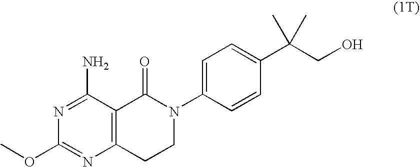

### 4-amino-6-[4-(2-hydroxy-1,1-dimethylethyl)phenyl]-2-methoxy-7,8-dihydropyrido[4,3-d]pyrimidin-5(6H)-one

Prepared analogous to (1A) from (2-(4-bromophenyl)-2-methylpropoxy)(tert-butyl)dimethylsilane which was prepared as follows:

2-(4-Bromophenyl)-2-methylpropanoic acid (1 g, 4 mmol) in tetrahydrofuran (15.5 ml)was cooled to 0° C. 1M Borane in tetrahydrofuran (4.11 ml, 4.11 mmol) was slowly added to the reaction mixture. Once addition was complete, the reaction was warmed up to room temperature and stirred for 18 hours. Aqueous solution of 1N hydrochloric acid and water were added and reation solution extracted with ethyl acetate. Organic washed with brine, dried over sodium sulfate, filtered and concentrated to give 2-(4-bromophenyl)-2-methylpropan-1-ol (0.9 g, 100%) as a clear oil.

2-(4-Bromophenyl)-2-methylpropan-1-ol (0.91 g, 4 mmol), tert-butyldimethylsilyl chloride (0.802 g, 5.16 mmol), and imidazole (0.541 g, 7.94 mmol) were combined in dimethylformamide (10 ml, 0.4M) at room temperature for 2 hours. Reaction diluted with water and extracted with ether. Organic layer washed with brine, dried over sodium sulfate, filtered and concentrated. Crude purified on silica gel eluting with a gradient from 0% to 5% ethyl acetate in heptane to give (2-(4-bromophenyl)-2-methylpropoxy)(tert-butyl)dimethylsilane (1.02 g, 75%) as a clear oil.

(1T): 1H NMR (400 MHz, DMSO-d6) δ ppm 8.35 (bs, 1H), 7.74 (bs, 1H), 7.35 (d, J=8.7, 2H), 7.22 (d, J=8.7, 2H), 4.66 (t, J=5.4, 1H), 3.83 (t, J=6.6, 2H), 3.82 (s, 3H), 3.39 (d, J=5.4, 2H), 2.91 (t, J=6.6, 2H), 1.20 (s, 6H)

m/z (M+1)=342.2

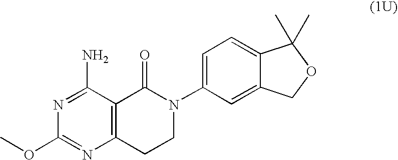

### 4-amino-6-(1,1-dimethyl-1,3-dihydro-2-benzofuran-5-yl)-2-methoxy-7,8-dihydropyrido[4,3-d]pyrimidin-5(6H)-one

Prepared analogous to (1A) from 5-bromo-1,1-dimethyl-1,3-dihydroisobenzofuran which was prepared as follows:

A solution of 5-bromoisobenzofuran-1(3H)-one (2.00 g, 9.4 mmol) in dry tetrahydrofuran (100 mL) under argon was cooled in an ice bath. Methylmagnesium bromide (9.3 mL, 28.0 mmol, 3M in diethylether) was added drop wise and the resulting mixture was left to warm to room temperature overnight. The reaction mixture was cooled to 0° C. and saturated aqueous ammonium chloride added. The mixture was extracted with ethyl acetate and the organics were dried over magnesium sulfate, filtered and concentrated. The crude product was filtered through a plug of silica with 50% ethyl acetate in heptane to give 2-(4-bromo-2-(hydroxymethyl)phenyl)propan-2-ol (1.90 g, 82%) was isolated as a colorless oil.

1H NMR (CDCl3, 400 MHz) δ ppm 7.45 (1H, s), 7.35 (1H, d), 7.14 (1H, d), 4.76 (2H, s) 1.64

Phosphoric acid (22.5 mL, 0.39 mol) was added to a suspension of 2-(4-bromo-2-(hydroxymethyl)phenyl)propan-2-ol (5.94 g, 24.2 mmol) in toluene (80 mL). The mixture was heated at 80° C. for 3 hours. The reaction was allowed to cool to room temperature then cooled to 0° C. The mixture was basified with 2M sodium hydroxide then extracted with ethyl acetate (x2), dried over magnesium sulfate, filtered and concentrated to give 5-bromo-1,1-dimethyl-1,3-dihydroisobenzofuran (5.42 g, 99%) as a clear oil.

1H NMR (CDCl3, 400 MHz) δ ppm 7.38 (1H, d), 7.32 (1H, s), 6.98 (1H, d), 5.02 (2H, s), 1.44 (6H, s). (6H, s).

(1U): 1H NMR (CDCl3, 400 MHz) δ ppm 8.32 (1H, s), 7.75 (1H, s), 7.24 (1H, s), 7.18 (2H,m), 4.91 (2H, s), 3.83 (4H, m), 2.92 (2H, t), 1.39 (6H, s).

m/z (M+1)=341.0

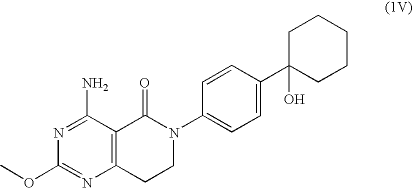

### 4-amino-6-[4-(1-hydroxycyclohexyl)phenyl]-2-methoxy-7,8-dihydropyrido[4,3-d]pyrimidin-5(6H)-one

Prepared analogous to (1A) from 1-(4-bromophenyl)cyclohexanol which was prepared as follows:

Dibromobenzene (3 g, 12.72 mmol) dissolved in tetrahydrofuran (35 mL) and cooled to −78° C. A 2.5M solution of n-butyllithium in hexane (5.6 mL, 14 mmol) added drop wise to cold reaction mixture and stirred at −78° C. for 1 hour. Cyclohexanone (1.45 mL, 14 mmol) was added drop wise at −78° C. Once addition was complete, reaction was warmed up to 0° C. for 1 hour. Saturated aqueous ammonium chloride and water added. Reaction mixture extracted with a 2:1 solution of ethyl acetate : heptane and organic layers washed with brine, dried over magnesium sulfate, filtered and concentrated to give 1-(4-bromophenyl)cyclohexanol (3.2 g, 98%) as a colorless oil.

1H NMR (400 MHz, CHLOROFORM-d) δ ppm 1.21-1.34 (m, 1H) 1.56-1.89 (m, 10H) 7.36 (d, 2H) 7.44 (d, 2H)

(1V): 1H NMR (DMSO-d6) Shift: δ ppm 8.38 (d, J=3.9 Hz, 1H), 7.78 (d, 1H), 7.50 (d, 2H), 7.26 (d, J=8.5 Hz, 2H), 4.70 (s, 1H), 3.83-3.91 (m, 5H), 2.95 (t, J=6.7 Hz, 2H), 1.57-1.80 (m, 8H), 1.46-1.53 (m, 1H), 1.20-1.31 (m, 1H)

m/z (M+1)=369.0

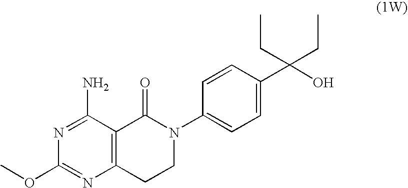

### 4-amino-6-[4-(1-ethyl-1-hydroxypropyl)phenyl]-2-methoxy-7,8-dihydropyrido[4,3-d]pyrimidin-5(6H)-one

Prepared analogous to (1A) from 3-(4-bromophenyl)pentan-3-ol which was prepared analogous to (1V) using pentan-3-one as the starting material.

(1W): 1H NMR (500 MHz, CHLOROFORM-d) δ ppm 0.78 (t, 6H) 1.77-1.91 (m, 5H) 3.06 (t, 2H) 3.93-3.99 (m, 5H) 5.65 (s, 1H) 7.29 (d, 2H) 7.43 (d, 2H) 8.63 (s, 1H)

m/z (M+1): 357.1

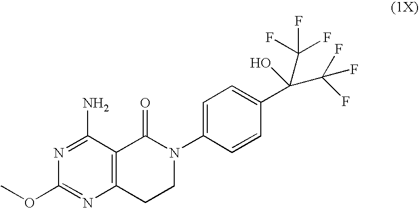

### 4-amino-2-methoxy-6-{4-[2,2,2-trifluoro-1-hydroxy-1-(trifluoromethyl)ethyl]phenyl}-7,8-dihydropyrido[4,3-d]pyrimidin-5(6H)-one

Prepared analogous to (1A) from tert-butyl(1,1,1,3,3,3-hexafluoro-2-(4-iodophenyl)propan-2-yloxy)dimethylsilane which was prepared as follows:

4-Iodobenzoic acid methyl ester (5 g, 19.08 mmol) dissolved in tetrahydrofuran (80 mL) and cooled to 0° C. (Trifluoromethyl)trimethylsilane (5.43 g, 38.2 mmol) and cesium fluoride (145 mg, 0.954 mmol) added. Once addition was complete, reaction was warmed up to room temperature and stirred for 3 hours. Additional (trifluoromethyl)trimethylsilane (2.715 g, 19.08 mmol) was added and reaction stirred at room temperature for 4 hours. 4 M aqueous solution of hydrochloric acid (20 mL) added and stirred for 5 hours. The reaction mixture was diluted with ethyl acetate (500 ml), washed with water (2x250 ml), dried over sodium sulfate, filtered and concentrated. Crude purified on silica gel eluting with a gradient from 0% to 10% ethyl acetate in heptane to afford 2,2,2-trifluoro-1-(4-iodophenyl)ethanone (1.8 g, 31%) GCMS=300 at 1.47 min and 1,1,1,3,3,3-hexafluoro-2-(4-iodophenyl)propan-2-ol (1.6 g, 22%); GCMS=370 at 1.60 min.

To a solution of tert-butyldimethylsilyl chloride (686 mg, 4.55 mmol), 4-dimethylaminopyridine (50.5 mg, 0.413 mmol) and triethylamine (0.864 mL, 6.2 mmol) in dichlromethane (20 mL), a solution of 1,1,1,3,3,3-hexafluoro-2-(4-iodophenyl)propan-2-ol in 5 ml of dichloromethane was added drop wise at room temperature. Stirred for 24 hours. Reaction was concentrated and water (50 mL) added. Solution extracted with ethyl acetate (100 mL) and organic layer washed with brine, dried over magnesium sulfate, filtered and concentrated. Crude purified on silica gel, eluting with 0% to10% ethyl acatate in heptane to give tert-butyl(1,1,1,3,3,3-hexafluoro-2-(4-iodophenyl)propan-2-yloxy)dimethylsilane (2 g, 99%) as a colorless oil.

1H NMR (400 MHz, CHLOROFORM-d) δ ppm 0.15 (s, 6H) 0.98 (s, 9H) 7.41 (d, J=8.78 Hz, 2H) 7.76 (d, J=8.98 Hz, 2H)

(1X): 1H NMR (500 MHz, DMSO-d6) δ ppm 2.92-3.01 (m, 2H) 3.86 (s, 3H) 3.96 (t, J=6.71 Hz, 2H) 7.52 (d, J=8.54 Hz, 2H) 7.70 (d, J=8.78 Hz, 2H) 7.85 (br. s., 1H) 8.33 (br. s., 1H) 8.75 (s, 1H)

m/z (M+1): 437.4

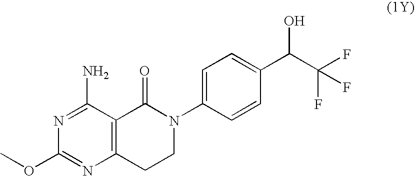

### 4-amino-2-methoxy-6-[4-(2,2,2-trifluoro-1-hydroxyethyl)phenyl]-7,8-dihydropyrido[4,3-d]pyrimidin-5(6H)-one

Prepared analogous to (1A) from 2,2,2-trifluoro-1-(4-iodophenyl)ethanol which was prepared as follows:

2,2,2-Trifluoro-1-(4-iodophenyl)ethanone (1.8 g, 6 mmol) was dissolved in methanol (60 mL) and cooled to 0° C. Sodium borohydride (0.227 g, 6 mmol) added and reaction stirred at 0° C. for 3 hours. Saturated aqueous ammonium chloride was added and the reaction mixture was extracted with ethyl acetate. Organic was washed with water (2 mL), dried over sodium sulfate, filtered and concentrated. Crude purified on silica gel eluting with a gradient from 3% to 20% ethyl acetate in heptane to give 2,2,2-trifluoro-1-(4-iodophenyl)ethanol (1.5 g, 82%). GCMS was 302 at 2.11 min.

1H NMR (400 MHz, CHLOROFORM-d) δ ppm 2.65 (d, J=4.49 Hz, 1H) 4.91-5.02 (m, 1H) 7.20 (d, J=8.39 Hz, 2H) 7.74 (d, J=8.39 Hz, 2H)

To a solution of tert-butyldimethylsilyl chloride (686 mg, 4.55 mmol), 4-dimethylaminopyridine (50.6 mg, 0.414 mmol) and triethylamine (0.865 mL, 6.21 mmol) in dichlromethane (20 mL), a solution of 2,2,2-trifluoro-1-(4-iodophenyl)ethanol in 5 ml of dichloromethane was added drop wise at room temperature. Stirred for 24 hours. Reaction was concentrated and water (50 mL) added. Solution extracted with ethyl acetate (100 mL) and organic layer washed with brine, dried over magnesium sulfate, filtered and concentrated. Crude purified on silica gel, eluting with 0% to10% ethyl acatate in heptane to give tert-butyldimethyl(2,2,2-trifluoro-1-(4-iodophenyl)ethoxy)silane (450 mg, 26%) as a colorless oil.

1H NMR (400 MHz, CHLOROFORM-d) δ ppm −0.03 (s, 3H) 0.10 (s, 3H) 0.88 (s, 9H) 4.84 (q, J=6.44 Hz, 1H) 7.15-7.18 (m, 1H) 7.18-7.20 (m, 1H) 7.68-7.71 (m, 1H) 7.71-7.74 (m, 1H).

(1Y): 1H NMR (400 MHz, METHANOL-d4) δ ppm 3.00 (t, J=6.83 Hz, 2H) 3.90-3.98 (m, 5H) 5.04 (q, J=7.09 Hz, 1H) 7.38 (d, J=8.59 Hz, 2H) 7.54 (d, J=8.59 Hz, 2H)

m/z (M+1)=369.2

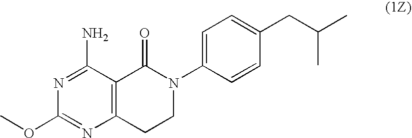

### 4-amino-6-(4-isobutylphenyl)-2-methoxy-7,8-dihydropyrido[4,3-d]pyrimidin-5(6H)-one

Prepared analogous to (1A) from 1-iodo-4-isobutylbenzene which was prepared as follows:

1-Isobutylbenzene (5 g, 37 mmol) was added to a mixture of iodine (9.46 g, 37.3 mmol) and silver(I) nitrite (5.85 g, 37.3 mmol) in dichloromethane (200 mL) at room temperature. Reaction was stirred for 96 hours. Yellow solid was filtered off and the filtrate was washed with 10% aqueous sodium sulfite (500 mL), saturated aqueous sodium bicarbonate and brine and dried over magnesium sulfate, filtered and concentrated. Crude was purified on silica gel, eluting with a gradient from 0% to 5% ethyl acetate in heptane to give 1-iodo-4-isobutylbenzene (7 g, 70%) as a pink oil.

(1Z): 1H NMR (400 MHz, CHLOROFORM-d) δ ppm 0.90 (d, J=6.64 Hz, 6H) 1.76-1.95 (m, 1H) 2.46 (d, J=7.03 Hz, 2H) 3.03 (t, J=6.83 Hz, 2H) 3.81-3.98 (m, 5H) 5.53 (br. s., 1H) 7.09-7.23 (m, 4H) 8.63 (br. s., 1H)

m/z (M+1)=327.2

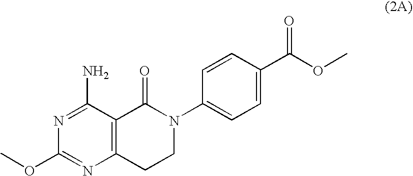

### Methyl 4-(4-amino-2-methoxy-5-oxo-7,8-dihydropyrido[4,3-d]pyrimidin-6(5H)-yl)benzoate

Prepared analogous to (1A) from commercially available methyl 4-bromobenzoate.

1H NMR (400 MHz, DMSO-d6) δ ppm 2.94 (t, J=6.73 Hz, 2H) 3.28 (s, 3H) 3.82 (s, 3H) 3.88-4.03 (m, 2H) 7.49 (d, J=8.78 Hz, 2H) 7.95 (d, J=8.78 Hz, 2H)

m/z (M+1)=329.1

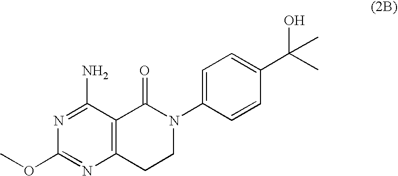

### 4-amino-6-[4-(1-hydroxy-1-methylethyl)phenyl]-2-methoxy-7,8-dihydropyrido[4,3-d]pyrimidin-5(6H)-one

(2A) (100 mg, 0.305 mmol) in tetrahydrofuran (4 ml) was cooled to −78° C. 3.0 M solution of methylmagnesium bromide in tetrahydrofuran (0.211 mL, 1.83 mmol) was added drop wise. Once addition was complete, reaction was warmed to room temperature for 2 hours.

Acetic acid (0.1 mL) added followed by saturated aqueous sodium bicarbonate (2 mL) and brine (5 mL). Solution extracted with ethyl acetate (5 mL), dried over magnesium sulfate, filtered and concentrated. Crude purified on silica gel, eluting with a gradient from 70% to 100% ethyl acetate in heptane to give the target compound (2B) (50 mg, 50%).

1H NMR (500 MHz, DMSO-d6) δ ppm 1.43 (s, 6H) 2.95 (t, 2H) 3.85 (s, 3H) 3.88 (t, 2H) 5.03 (s, 1H) 7.26 (d, 2H) 7.48 (d, 2H) 7.78 (d, 1H) 8.38 (d, 1H)

m/z (M+1)=329.5

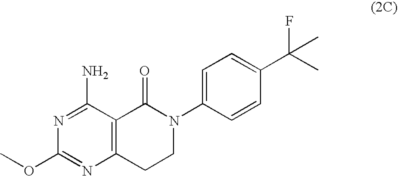

### 4-amino-6-[4-(1-fluoro-1-methylethyl)phenyl]-2-methoxy-7,8-dihydropyrido[4,3-d]pyrimidin-5(6H)-one

(2B) (41 mg, 0.12 mmol) in dichloromethane (4 mL) was cooled to −78° C. Deoxo-fluor® (0.035 mL, 0.188 mmol) was added drop wise to cold reaction mixture. Once addition was complete, the reaction was warmed to room temperature and stirred for 18 hours. Saturated aqueous sodium bicarbonate (4 mL) wad added and stirred vigorously for 30 minutes. Reaction mixture was extracted with dichloromethane (5 mL) dried over sodium sulfate, filtered and concentrated. Crude purified on silica gel, eluting with a gradient from 60% to 90% ethyl acetate in heptane to give (2C) (33 mg, 80%).

1H NMR (500 MHz, CHLOROFORM-d) δ ppm 1.71 (d, 6H) 3.08 (t, 2H) 3.95 (t, 2H) 3.97 (s, 3H) 5.60 (s, 1H) 7.32 (d, 2H) 7.45 (d, 2H) 8.64 (s, 1H)

19F NMR (376 MHz, CHLOROFORM-d) δ ppm −137.82-−137.66 (m, 1 F)

m/z (M+1)=331.5

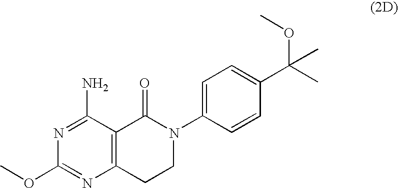

### 4-amino-2-methoxy-6-[4-(1-methoxy-1-methylethyl)phenyl]-7,8-dihydropyrido[4,3-d]pyrimidin-5(6H)-one

(2B) (26 mg, 0.079 mmol) in dichloromethane (3 mL). Thionyl chloride (0.4 mL, 5 mmol) was added in one portion at room temperature and the mixture was stirred for 2 hours. Reaction was then cooled to 10° C. and methanol (1 mL) was added. The reaction mixture was concentrated and the resulting residue was dissolved back in methanol (4 mL) and stirred at room temperature for 24 hours. Reaction concentrated and saturated aqueous sodium bicarbonate was added. Reaction extracted with ethyl acetate, dried over sodium sulfate, filtered and concentrated. Crude purified on silica gel, eluting with a gradient from 60% to 90% ethyl acetate in heptane to give the target compound (2D) (15 mg, 56%).

1H NMR (500 MHz, CHLOROFORM-d) δ ppm 1.55 (s, 3H) 3.08 (t, 2H) 3.11 (s, 3H) 3.11 (s, 3H) 3.97 (t, 2H) 3.97 (s, 3H) 5.60 (s, 1H) 7.31 (d, 2H) 7.47 (d, 2H) 8.64 (s, 1H)

m/z (M+1)=343.5

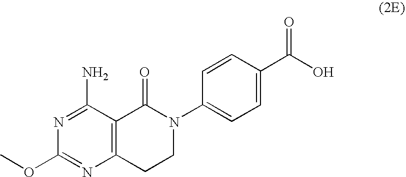

### 4-(4-amino-2-methoxy-5-oxo-7,8-dihydropyrido[4,3-d]pyrimidin-6(5H)-yl)benzoic acid

(2A) (174 mg, 0.530 mmol) was dissolved in tetrahydrofuran (6.25 mL), methanol (4 mL) and water (2.13 mL). Lithium hydroxide (89.0 mg, 2.12 mmol) was added and reaction was stirred at room temperature for 16 hours. Reaction was heated to 40° C. for 4 hours and reaction was concentrated to dryness. Crude was dissolved in minimal amounts of water and acidified with 1N hydrochloric acid. The resulting precipitate was filtered off and dried in a vacuum oven to give the title compound, (2E) (154 mg, 92%) as an off-white solid.

1H NMR (400 MHz, DMSO-d6) δ ppm 2.94 (t, J=6.64 Hz, 2H) 3.82 (s, 3H) 3.93 (t, J=6.73 Hz, 2H) 7.45 (d, J=8.78 Hz, 2H) 7.92 (d, J=8.78 Hz, 2H)

m/z (M+1)=315.1

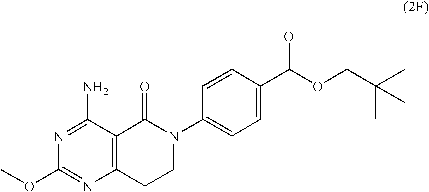

### 2,2-dimethylpropyl 4-(4-amino-2-methoxy-5-oxo-7,8-dihydropyrido[4,3-d]pyrimidin-6(5H)-yl)benzoate

(2E) (100 mg, 0.318 mmol), neopentyl alcohol (280 mg, 3.18 mmol) and catalytic 4-dimethylaminopyridine (4 mg, 0.032 mmol) in dimethylformamide (1.59 ml, 0.2M) was cooled to 0° C. Diisopropylcarbodiimide (0.059 ml, 0.382 mmol) was added and stirred at room temperature for 16 hours. Reaction was partitioned between water and ethyl acetate. Layers separated and organic layer washed with water, brine and dried over sodium sulfate, filtered and concentrated. Crude product purified on silica gel eluting with a gradient system: 50% ethyl acetate in heptane to 100% ethyl acetate to give the target compound, (2F)(29 mg, 24%) as a white solid.

1H NMR (400 MHz, DMSO-d6) δ ppm 0.97 (s, 9H) 2.94 (t, J=6.64 Hz, 2H) 3.79-3.85 (m, 3H) 3.90-3.98 (m, 4H) 7.50 (d, J=8.78 Hz, 2H) 7.97 (d, J=8.78 Hz, 2H)

m/z (M+1)=385.1

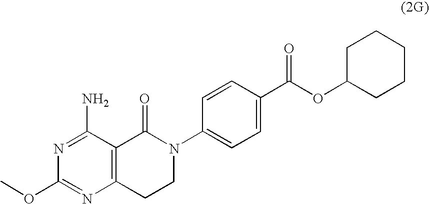

### Cyclohexyl 4-(4-amino-2-methoxy-5-oxo-7,8-dihydropyrido[4,3-d]pyrimidin-6(5H)-yl)benzoate

Prepared analogous to (2F) from the commercially available cyclohexanone.

(2G): 1H NMR (500 MHz, DMSO-d6) δ ppm 1.29-1.47 (m, 4H) 1.48-1.62 (m, 2H) 1.67-1.79 (m, 2H) 1.87 (d, J=8.54 Hz, 2H) 2.98 (t, J=6.59 Hz, 2H) 3.86 (s, 3H) 3.97 (t, J=6.71 Hz, 2H) 4.85-4.99 (m, 1H) 7.51 (d, J=8.78 Hz, 2H) 7.98 (d, J=8.54 Hz, 2H)

m/z (M+1)=397.1

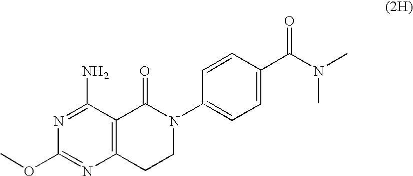

### 4-(4-amino-2-methoxy-5-oxo-7,8-dihydropyrido[4,3-d]pyrimidin-6(5H)-yl)-N,N-dimethylbenzamide

(2E) (20 mg, 0.06 mmol) was dissolved in dichloromethane (0.64 ml, 0.1M) and dimethylformamide(0.001 ml, 0.001 mmol) and cooled to 0° C. Oxalyl chloride (0.007 ml, 0.077 mmol) was added slowly and stirred for 30 minutes at 0° C. Reaction slowly allowed to warm up to room temperature and dimethylamine (0.320 mL, 0.640 mmol) added and stirred for 16 hours. Reaction concentrated and crude product purified on silica gel eluting with 10% methanol in dichloromethane to give the target compound (2H) (4.5 mg, 20%) as a white solid.

1H NMR (500 MHz, DMSO-d6) δ ppm 2.97 (t, 2H) 2.97 (br. s., 6H) 3.86 (s, 3H) 3.94 (t, J=6.71 Hz, 2H) 7.43 (q, J=8.54 Hz, 4H)

m/z (M+1)=342.1

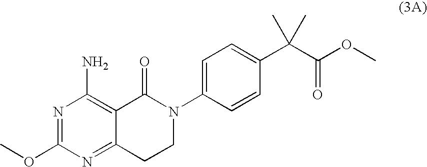

### methyl 2-[4-(4-amino-2-methoxy-5-oxo-7,8-dihydropyrido[4,3-d]pyrimidin-6(5H)-yl)phenyl]-2-methylpropanoate

Prepared analogous to (1A) using methyl 2-(4-bromophenyl)-2-methylpropanoate as the starting bromide which was synthesized as follows:

A solution of 4-bromophenylacetic acid (10 g, 47 mmol) in methanol (194 ml, 46.5M) and sulfuric acid (2.48 ml, 46.5 mmol) was heated to reflux for 16 hours. Reaction was concentrated, diluted with ethyl acetate and washed with saturated sodium bicarbonate and brine. Organic was dried over sodium sulfate, filtered and concentrated to give methyl 2-(4-bromophenyl)acetate (10.63 g ,100%) as a colorless oil.

1H NMR (400 MHz, CHLOROFORM-d) δ ppm 3.56 (s, 2H) 3.68 (s, 3H) 7.14 (d, J=8.59 Hz, 2H) 7.43 (d, J=8.59 Hz, 2H)

A solution of methyl 2-(4-bromophenyl)acetate (6 g, 30 mmol) in tetrahydrofuran (67.2 ml, 0.39M) was added 1M potassium t-butoxide in tetrahydrofuran (57.6 ml, 57.6 mmol). Reaction mixture was cooled to 0° C. and methyl iodide (3.59 ml, 57.6 mmol) was added drop wise. After addition was complete, reaction was slowly warmed up to room temperature and stirred for 16 hours. Reaction mixture was then carefully quenched with 1M hydrochloric acid and concentrated. Reaction was diluted with water and extracted with ethyl acetate. Pooled organics were washed with water and brine and then dried over sodium sulfate, filtered and concentrated to give a crude dark oil. Crude product purified on silica gel eluting with 0%-5% ethyl acetate in heptane to give methyl 2-(4-bromophenyl)-2-methylpropanoate (3A-1) (6.44 g, 92%) as a yellow oil

1H NMR (400 MHz, CHLOROFORM-d) δ ppm 1.54 (s, 6H) 3.63 (s, 3H) 7.19 (d, J=8.78 Hz, 2H) 7.43 (d, J=8.98 Hz, 2H)

m/z (M+1)=371.2

(3A): 1H NMR (400 MHz, DMSO-d6) δ ppm 2.94 (t, J=6.73 Hz, 2H) 3.28 (s, 3H) 3.82 (s, 3H) 3.88-4.03 (m, 2H) 7.49 (d, J=8.78 Hz, 2H) 7.95 (d, J=8.78 Hz, 2H)

m/z (M+1)=329.1

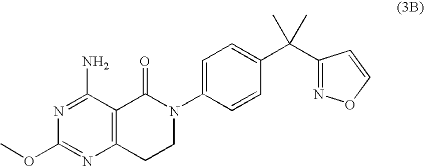

### 4-amino-6-[4-(1-isoxazol-3-yl-1-methylethyl)phenyl]-2-methoxy-7,8-dihydropyrido[4,3-d]pyrimidin-5(6H)-one

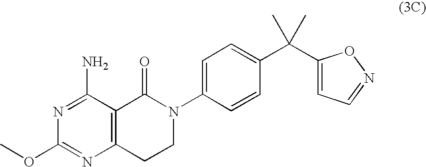

### 4-amino-6-[4-(1-isoxazol-5-yl-1-methylethyl)phenyl]-2-methoxy-7,8-dihydropyrido[4,3-d]pyrimidin-5(6H)-one

To a solution of (3A) (10 g, 27 mmol) in tetrahydrofuran (250 mL) and dichloromethane (250 mL) was added lithium borohydride (1.2 g, 54 mmol) and methanol (2.2 ml, 54 mmol).

The reaction mixture was stirred at room temperature for 60 hours. Reaction poured onto ice-water (500 mL) and acidified with 10% aqueous citric acid and extracted with dichloromethane (3×500 mL), dried over magnesium sulfate and concentrated. The residue was purified by column chromatography, eluting with a gradient from 50% ethyl acetate in hexane to 50% methanol in ethyl acetate to 50% methanol in dichloromethane to give 4-amino-6-(4-(1-hydroxy-2-methylpropan-2-yl)phenyl)-2-methoxy-7,8-dihydropyrido[4,3-d]pyrimidin-5(6H)-one (2.5 g , 26%).

1H NMR (400 MHz, DMSO-d6) δ ppm 8.35 (br. s, 1H), 7.74 (br. s, 1H), 7.34 (d, 2H), 7.21 (d, 2H), 4.67 (t, 1H), 3.83 (m, 5H), 3.39 (d, 2H), 2.92 (t, 2H), 1.14 (s, 6H).

To a solution of give 4-amino-6-(4-(1-hydroxy-2-methylpropan-2-yl)phenyl)-2-methoxy-7,8-dihydropyrido[4,3-d]pyrimidin-5(6H)-one (1.48 g, 4.32 mmol) in dichloromethane (30 mL) was added Dess-Martin Periodinane (2.20 g, 5.19 mmol). The reaction mixture was stirred for 2 hours at room temperature and then quenched with 10% aqueous sodium thiosulfate. After stirring a further 30 min, the layers were separated and the organics washed with saturated aqueous sodium bicarbonate, dried over sodium sulfate and concentrated to give 2-(4-(4-amino-2-methoxy-5-oxo-7,8-dihydropyrido[4,3-d]pyrimidin-6(5H)-yl)phenyl)-2-methylpropanal (1.6 g., >85% pure by NMR) which was used without further purification.

1H NMR (400 MHz, DMSO-d6) δ ppm 9.49 (s, 1H), 8.32 (br. s, 1H), 7.78 (br. s, 1H), 7.32 (d, 2H), 7.30 (d, 2H), 3.86 (t, 2H), 3.82 (s, 3H), 2.92 (t, 2H), 1.38 (s, 6H).

To a solution of 2-(4-(4-amino-2-methoxy-5-oxo-7,8-dihydropyrido[4,3-d]pyrimidin-6(5H)-yl)phenyl)-2-methylpropanal (2.0 g, 5.88 mmol) in tetrahydrofuran (30 mL) at −78° C. was slowly added a solution of ethynyl magnesiumbromide (0.5M in diethyl ether, 24 mL, 11.76 mmol). The reaction mixture was stirred for 16 hours allowing to warm to room temperature and then quenched with water (20 ml). The aqueous was extracted with ethyl acetate (3×20 mL), and the combined organics washed with brine (20 mL), dried over sodium sulfate, concentrated and purified by column chromatography, eluting with a gradient from 1% to 10% methanol in dichloromethane giving 4-amino-6-(4-(3-hydroxy-2-methylpent-4-yn-2-yl)phenyl)-2-methoxy-7,8-dihydropyrido[4,3-d]pyrimidin-5(6H)-one (0.90 g, 42%) as an off-white solid.

1H NMR (400 MHz, DMSO-d6) δ ppm 8.35 (br. s, 1H), 7.75 (br. s, 1H), 7.37 (d, 2H), 7.22 (d, 2H), 5.48 (d, 1H), 4.31 (d, 1H), 3.84 (t, 2H), 3.81 (s, 3H), 3.16 (dd, 1H), 2.92 (t, 2H), 1.28 (s, 6H).

To a solution of 4-amino-6-(4-(3-hydroxy-2-methylpent-4-yn-2-yl)phenyl)-2-methoxy-7,8-dihydropyrido[4,3-d]pyrimidin-5(6H)-one (0.60 g, 1.62 mmol) in dichloromethane (12 mL) was added Dess-Martin Periodinane (0.97 g, 2.30 mmol). The reaction mixture was stirred for 4 hours at room temperature and then quenched with 10% aqueous sodium thiosulfate. After stirring a further 30 minutes, the layers were separated and the organics washed with saturated aqueous sodium bicarbonate, dried over sodium sulfate and concentrated to give 4-amino-2-methoxy-6-(4-(2-methyl-3-oxopent-4-yn-2-yl)phenyl)-7,8-dihydropyrido[4,3-d]pyrimidin-5(6H)-one (0.46 g, 78%) which was used without further purification.

1H NMR (400 MHz, CDCl3) δ ppm 8.60 (br. s, 1H), 7.34 (d, 2H), 7.32 (d, 2H), 5.53 (br. s, 1H), 3.94 (t, 2H), 3.94 (s, 3H), 3.11 (s, 1H), 3.05 (t, 2H), 1.59 (s, 6H).

To a solution of 4-amino-2-methoxy-6-(4-(2-methyl-3-oxopent-4-yn-2-yl)phenyl)-7,8-dihydropyrido[4,3-d]pyrimidin-5(6H)-one (458 mg, 1.26 mmol) in ethanol (12 mL) was added hydroxylamine hydrochloride (87 mg, 1.26 mmol). The reaction mixture was stirred at reflux for 16 hours. Reaction cooled to room temperature and concentrated. The residue was purified by column chromatography eluting with ethyl acetate in heptane to give a 2:1 mixture of the 2 isomers. The isomers were separated by HPLC (column: X-bridge C18 5?m, 30×150 mm; isocratic method: 25% acetonitrile/formic acid aq. 0.1%; flow rate: 50 mL/min) to give the target compounds (3B) (10 mg, 2.1%) and (3C) (6 mg, 1.3%).

(3B): 1H NMR (400 MHz, CDCl3) δ ppm 8.60 (br. s, 1H), 8.38 (s, 1H), 7.31 (d, 2H), 7.25 (d, 2H), 6.04 (s, 1H), 5.55 (br. s, 1H), 3.94 (s, 3H), 3.93 (t, 2H), 3.05 (t, 2H), 1.74 (s, 6H).

m/z (M+1)=380.0

(3C): 1H NMR (400 MHz, CDCl3) δ ppm 8.58 (br. s, 1H), 8.14 (s, 1H), 7.29 (d, 2H), 7.25 (d, 2H), 6.01 (s, 1H), 5.59 (br. s, 1H), 3.94 (s, 3H), 3.93 (t, 2H), 3.05 (t, 2H), 1.74 (s, 6H).

m/z (M+1)=380.0

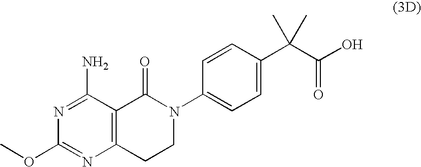

### 2-[4-(4-amino-2-methoxy-5-oxo-7,8-dihydropyrido[4,3-d]pyrimidin-6(5H)-yl)phenyl]-2-methylpropanoic acid

(3A) (424 mg, 1.14 mmol) was dissolved in tetrahydrofuran (13.5 mL), methanol (8.8 mL) and water (4.6 mL). Lithium hydroxide (192 mg, 4.58 mmol) was added and reaction was stirred at room temperature for 36 hours. Reaction was concentrated to dryness and diluted with a small amount of water, acidified with aqueous 1N hydrochloric acid. Precipitate formed and filtered off to give the target compound, (3D) (370 mg, 90%) as an off white solid.

1H NMR (400 MHz, DMSO-d6) δ ppm 1.44 (s, 6H) 2.91 (t, J=6.73 Hz, 2H) 3.82 (s, 3H) 3.85 (t, J=6.73 Hz, 2H) 7.26 (d, 2H) 7.33 (d, 2H)

m/z (M+1)=357.1

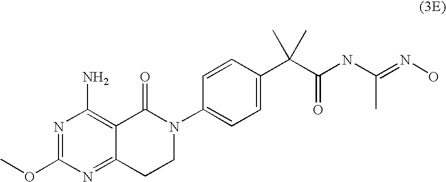

### (E)-2-(4-(4-amino-2-methoxy-5-oxo-7,8-dihydropyrido[4,3-d]pyrimidin-6(5H)-yl)phenyl)-N-(1-(hydroxyimino)ethyl)-2-methylpropanamide

To a mixture of (3D) (100 mg, 0.281 mmol) at 0° C. was added thionyl chloride (0.5 mL) and 1 drop of dimethylformamide. The reaction was then warmed up to room temperature and stirred for 5 hours. The reaction mixture was concentrated to dryness under reduced pressure to remove excess of thionyl chloride and the crude mixture was dissolved in tetrahydrofuran (15 mL) and n-hydroxy-acetamidine (62.5 mg, 0.843 mmol) and molecular sieves (4 Å beads) were added. Reaction stirred at room temperature for 16 hours. Reaction filtered and washed with methanol. Filtrate concentrated, dissolved in ethyl acetate and washed with water, dried over magnesium sulfate, filtered and concentrated.

Crude was purified on silica gel eluting with 1% to 5% methanol in dichloromethane to give (3E) (80 mg, 69%) as a yellow solid.

1H NMR (400 MHz, CHLOROFORM-d) δ ppm 1.63 (s, 6H) 1.88 (s, 3H) 3.03 (t, J=6.73 Hz, 2H) 3.83-3.97 (m, 5H) 4.43 (br. s., 2H) 5.63 (br. s., 1H) 7.20-7.29 (m, 2H) 7.42 (d, J=8.78 Hz, 2H) 8.56 (br. s., 1H)

m/z (M+1)=413.1

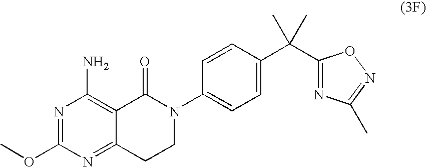

### 4-amino-2-methoxy-6-{4-[1-methyl-1-(3-methyl-1,2,4-oxadiazol-5-yl)ethyl]phenyl}-7,8-dihydropyrido[4,3-d]pyrimidin-5(6H)-one

(3E) (80 mg, 0.19 mmol) was dissolved in dimethylacetamide with 4 Å molecular sieves. Reaction heated to 140° C. for 5 hours. Reaction filtered and solids washed with dichloromethane. Filtrate washed with brine, dried over magnesium sulfate and concentrated to dryness. Crude product was purified on silica gel eluting with a gradient: 1-5% methanol in dichloromethane to give the target compound (3F) (30 mg, 39%) as a white solid

1H NMR (400 MHz, CHLOROFORM-d) δ ppm 1.81 (s, 5H) 2.37 (s, 3H) 3.02 (t, J=6.73 Hz, 2H) 3.86-3.95 (m, 5H) 5.61 (br. s., 1H) 7.22-7.29 (m, 2H) 7.30-7.37 (m, 2H) 8.56 (br. s., 1H)

m/z (M+1)=395.1

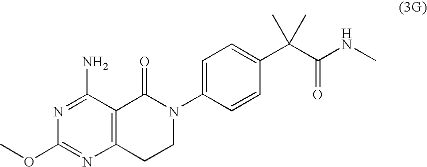

### 2-[4-(4-amino-2-methoxy-5-oxo-7,8-dihydropyrido[4,3-d]pyrimidin-6(5H)-yl)phenyl]-N,2-dimethylpropanamide

(3D) (7 mg, 0.02 mmol) was dissolved in thionyl chloride (2 mL) at room temperature and stirred for 1 hour. The reaction was concentrated to dryness and toluene (2×5 mL) was added and concentrated to dryness. The residue was dried under vacuum for 2 hours. The residue was then dissolved in methylamine (2M solution in tetrahydrofuran) and stirred at room temperature for 16 hours. Reaction was concentrated and purified on silica gel eluting with 1% to 3% to 5% methanol in dichloromethane to give target compound (3G) (3.5 mg, 50%) as a white powder.

1H NMR (400 MHz, DMSO-d6) δ ppm 8.33 (bs, 1H), 7.75 (bs, 1H), 7.34 (q, J=4.3, 1H), 7.28 (d, J=8.8, 2H), 7.24 (d, J=8.8, 2H), 3.84 (t, J=6.8, 2H), 3.82 (s, 3H), 2.91 (t, J=6.8, 2H), 2.52 (d, J=4.5, 3H), 1.41 (s, 6H)

m/z (M+1)=370.1

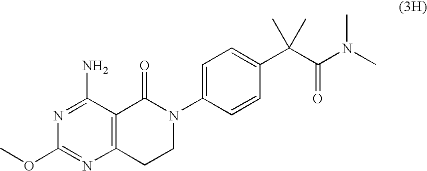

### 2-[4-(4-amino-2-methoxy-5-oxo-7,8-dihydropyrido[4,3-d]pyrimidin-6(5H)-yl)phenyl]-N,N,2-trimethylpropanamide

(3D)(7 mg, 0.02 mmol) was dissolved in thionyl chloride (2 mL) at room temperature and stirred for 1 hour. The reaction was concentrated to dryness and toluene (2×5 mL) was added and concentrated to dryness. The residue was dried under vacuum for 2 hours. The residue was then dissolved in dimethylamine (2M solution in tetrahydrofuran) and stirred at room temperature for 16 hours. Reaction was concentrated and purified on silica gel eluting with 1% to 5% to 10% methanol in dichloromethane to give target compound (3H) (6 mg, 80%) as an off white powder.

1H NMR (400 MHz, DMSO-d6) δ ppm 8.33 (bs, 1H), 7.75 (bs, 1H), 7.31 (d, J=8.7, 2H), 7.17 (d, J=8.7, 2H), 3.85 (t, J=6.8, 2H), 3.81 (s, 3H), 3.28 (s, 6H), 2.91 (t, J=6.8, 2H), 1.41 (s, 6H)

m/z (M+1)=384.1

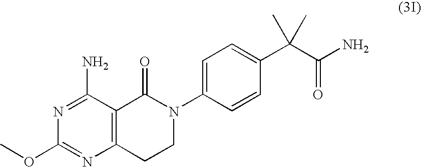

### 2-[4-(4-amino-2-methoxy-5-oxo-7,8-dihydropyrido[4,3-d]pyrimidin-6(5H)-yl)phenyl]-2-methylpropanamide

To a solution of methyl 4-nitrophenylacetate (100 g, 512 mmol) and methyliodide (128 mL, 205 mol) in tetrahydrofuran (500 mL) and dimethylformamide (100 mL) was added drop wise a solution of potassium t-butoxide (50.0 g, 1.53 mol) in tetrahydrofuran (500 mL). The reaction temperature was maintained at 50° C. during the addition. A second portion of potassium t-butoxide (50 g) was added and the mixture was heated at 50° C. for 1 hour. The mixture was cooled to room temperature and poured into 2M hydrochloric acid (1 L) with stirring. Toluene (1 L) was added, and the organic layer was washed with water (500 mL), brine (500 mL), and dried over magnesium sulfate, filtered and concentrated to afford a red oil. Hexane (200 mL) was added and upon standing at room temperature overnight, the product crystallized and was filtered and dried to give methyl 2-methyl-2-(4-nitrophenyl)propanoate (90 g, 79%).

1H NMR (CDCl3), 400 MHz): δ ppm 8.15 (d, 2H), 7.50 (d, 2H), 3.67 (s, 3H), 1.61 (s, 6H).

To a solution of methyl 2-methyl-2-(4-nitrophenyl)propanoate (50.0 g, 0.224 mol) in methanol (300 mL) was added 5% palladium on carbon (2.5 g, wet). The mixture was stirred for 18 h under 60 bar of hydrogen. Reaction filtered through a pad of celite. The filtrate was concentrated, and the product was recrystallized from 2:1 toluene:hexane to give methyl 2-(4-aminophenyl)-2-methylpropanoate (20.8 g, 48%).

1H NMR (CDCl3, 400 MHz): δ ppm 7.12 (d, 2H), 6.65 (d, 2H), 3.61 (s, 3H), 1.53 (s, 6H).

2-(4-Aminophenyl)-2-methylpropanoate (190 g, 986 mmol) was dissolved in ethyl acrylate (118 mL, 1084 mmol) and acetic acid (60 mL, 1025 mmol) was added. The mixture was heated to 70° C. with mechanical stirring for 12 hours. The reacting mixture was cooled to room temperature and diluted with toluene (125 mL) and 10% aqueous potassium carbonate (125 mL). After stirring for 1 hour, the organic layer was washed with brine, dried over sodium sulfate, filtered and concentrated to a dark oil, which solidified under vacuum to a low melting solid ethyl N-[4-(2-methoxy-1,1-dimethyl-2-oxoethyl)phenyl]-beta-alaninate (279 g) which was used in the next step without further purification.

Ethyl N-[4-(2-methoxy-1,1-dimethyl-2-oxoethyl)phenyl]-beta-alaninate was dissolved in ethyl acetate (1.35 L). Cyanoacetic acid (80.9 g, 950 mmol) and triethylamine (400 mL, 2.85 mol) were then added sequentially, and the mixture was cooled to 0° C. A 50% solution of propanephosphonic cyclic anhydride in ethyl acetate (628 mL, 1.045 mol) was added drop wise over 20 minutes, at a rate such that the internal reaction temperature did not exceed 10° C. After warming to room temperature over 30 minutes, the mixture was diluted with ethyl acetate (400 mL) and washed sequentially with 10% aqueous potassium phosphate (900 mL), 1 N hydrochloric acid (1.8 L), and brine (900 mL). The organic layer was dried over sodium sulfate)and concentrated to 304 g of a low melting pale yellow solid ethyl N-(cyanoacetyl)-N-[4-(2-methoxy-1,1-dimethyl-2-oxoethyl)phenyl]-beta-alaninate which was used in the next step without further purification.

Ethyl N-(cyanoacetyl)-N-[4-(2-methoxy-1,1-dimethyl-2-oxoethyl)phenyl]-beta-alaninate (151 g) was dissolved in methanol (1.7 L) and 1,8-diazaobicyclo[5.4.0]undec-7-ene (76 mL, 502 mmol) was added. The mixture was heated to 70° C. for 2 hours. Reacton concentrated and ethyl acetate (750 mL) and 1N hydrochloric acid (750 mL) were added with stirring. Heptane (750 mL) was slowly added over 30 minutes, inducing the precipitation of a well dispersed solid. After stirring for 30 minutes at room temperature, the mixture was filtered, and the solid cake was washed with water (100 mL) and 1:1 heptane:ethyl acetate (250 mL), and dried under vacuum at 50° C. to give methyl 2-(4-(3-cyano-4-hydroxy-2-oxo-5,6-dihydropyridin-1(2H)-yl)phenyl)-2-methylpropanoate (120 g, 77% over 3 stetps) as an off-white powder.

¹H NMR (400 MHz, DMSO-d₆) δ ppm 1.47 (s, 6H) 2.77 (t, J=6.73 Hz, 2H) 3.56 (s, 3H) 3.75 (t, J=6.83 Hz, 2H) 7.17-7.21 (m, 2H) 7.24-7.29 (m, 2H)

m/z (M+1)=315.2

Methyl 2-(4-(3-cyano-4-hydroxy-2-oxo-5,6-dihydropyridin-1(2H)-yl)phenyl)-2-methylpropanoate (50.0 g, 159 mmol) in dimethylformamide (7.6 mL, 98.6 mmol), and dichloromethane (625 mL) was cooled to 0° C. Oxalyl chloride (25.5 mL, 294 mmol) was added drop wise over 20 minutes, and the reaction mixture was warmed to room temperature over 1 hour. Methanol (725 mL) was then added via addition funnel at room temperature, and the mixture was heated at reflux overnight. The dichloromethane was then removed under reduced pressure to afford a thick slurry, which was filtered. The white solid collected was washed with methanol (250 mL) and dried under vacuum at 50° C. to give methyl 2-(4-(3-cyano-4-methoxy-2-oxo-5,6-dihydropyridin-1(2H)-yl)phenyl)-2-methylpropanoate (52.2 g, 87%) as an off-white powder.

¹H NMR (400 MHz, DMSO-d₆) δ ppm 1.47 (s, 6H) 3.02 (t, J=6.83 Hz, 2H) 3.56 (s, 3H) 3.82 (t, J=6.83 Hz, 2H) 4.01 (s, 3H) 7.20-7.25 (m, 2H) 7.26-7.30 (m, 2H)

m/z (M+1)=329.5

Methyl 2-(4-(3-cyano-4-methoxy-2-oxo-5,6-dihydropyridin-1(2H)-yl)phenyl)-2-methylpropanoate (15.0 g, 45.7 mmol) was suspended in methanol (150 mL) at 0° C. and cyanamide (4.20 g, 100.5 mmol) was added. Sodium methoxide (34.5 mL of 25% w/w solution in methanol, 150.7 mmol) was added drop wise and the mixture was allowed to reach room temperature over 1 hour. This mixture of intermediate cyanamide adduct was then acidified by the addition of sulfuric acid and heated at 65° C. for 2.5 h. The mixture was cooled to 0° C. and basified by the drop wise addition of aqueous sodium hydroxide (1N). The resulting slurry was stirred at room temperature for 30 min and filtered. The cake was washed with water and dried under vacuum at 60° C. to give methyl 2-(4-(4-amino-2-methoxy-5-oxo-7,8-dihydropyrido[4,3-d]pyrimidin-6(5H)-yl)phenyl)-2-methylpropanoate (3A) (13.63 g, 83%) as a white solid. (3A) contaminated with up to 10% of byproduct vinylogous amide. An analytical sample gave:

¹H NMR (400 MHz, CHLOROFORM-d) δ ppm 1.57 (s, 6H) 3.03 (t, J=6.83 Hz, 2H) 3.64 (s, 3H) 3.89-3.96 (m, 5H) 5.56 (br. s., 1H) 7.26 (d, J=8.98 Hz, 2H) 7.37 (d, J=8.78 Hz, 2H) 8.59 (br. s., 1H)

m/z (M+1)=371.2

(3A) (15.0 g, 40.5 mmol) was suspended in tetrahydrofuran (330 mL) at room temperature, and potassium trimethylsilanolate (17.3 g, 121.5 mmol) was added. The thick suspension was heated at reflux overnight. Reaction volume was concentrated to 50% of total volume and hexane (75 mL) was added. After stirring the slurry for 30 minutes, the mixture was filtered and the cake washed with water and 1:1 hexane:ethyl acetate (75 mL) and dried under vacuum to give (3D) (13.28 g, 92%) as a pale yellow solid.

1H NMR (400 MHz, DMSO-d6) δ ppm 1.44 (s, 6H) 2.91 (t, J=6.73 Hz, 2H) 3.82 (s, 3H) 3.85 (t, J=6.73 Hz, 2H) 7.26 (d, 2H) 7.33 (d, 2H)

m/z (M+1)=357.1

(3D) (15.0 g, 42.1 mmol) was suspended in dimethylformamide (75 mL) and 1,1′-carbonyldiimidazole (8.2 g, 50.5 mmol) was added. After stirring for 45 minutes, ammonium hydroxide (28% solution; 100 mL) was added in one portion. The thick suspension was stirred at room temperature for 2 hours then filtered and washed with water. The product was dried under vacuum at 60° C. to give the title compound 2-[4-(4-amino-2-methoxy-5-oxo-7,8-dihydropyrido[4,3-d]pyrimidin-6(5H)-yl)phenyl]-2-methylpropanamide (3I) (12.9 g, 86%) as an off-white solid.

1H NMR (500 MHz, DMSO-d6) δ ppm 1.45 (s, 6H) 2.95 (t, J=6.83 Hz, 2H) 3.85 (s, 3H) 3.88 (t, J=6.71 Hz, 2H) 6.90 (s, 1H) 6.94 (s, 1H) 7.29 (d, 2H) 7.36 (d, 2H) 7.79 (d, J=3.90 Hz, 1H) 8.37 (d, J=3.90 Hz, 1H).

m/z (M+1)=356.0

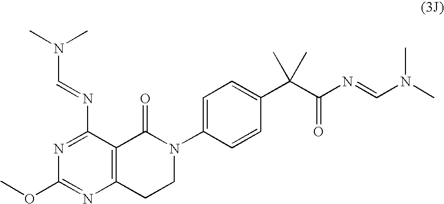

### (E)-N-((dimethylamino)methylene)-2-(4-(4-(4(E)-(dimethylamino)methyleneamino)-2-methoxy-5-oxo-7,8-dihydropyrido[4,3-d]pyrimidin-6(5H)-yl)phenyl)-2-methylpropanamide

(3I) (110 mg, 0.310 mmol) suspended in dimethylformamide dimethyl acetal (5 mL, 35.12 mmol). Reaction mixture heated to 80° C. for 72 hours. Reaction was concentrated to give (3J) (138 mg, 95%) which was used for the next step without further purification.

1H NMR (500 MHz, DMSO-d6) δ ppm 1.48 (s, 6H) 2.91 (s, 3H) 2.95 (t, 2H) 2.99 (s, 3H) 3.07 (s, 3H) 3.13 (s, 3H) 3.85 (t, 2H) 3.89 (s, 3H) 7.24 (d, 2H) 7.32 (d, 2H) 8.32 (s, 1H) 8.42 (s, 1H)

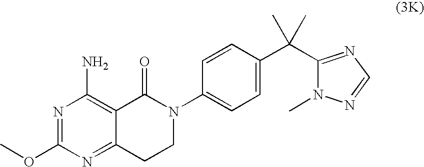

### 4-amino-2-methoxy-6-{4-[1-methyl-1-(1-methyl-1H-1,2,4-triazol-5-yl)ethyl]phenyl}-7,8-dihydropyrido[4,3-d]pyrimidin-5(6H)-one

(3J) (30 mg, 0.064 mmol) and methyl hydrazine (29.5 mg, 0.640 mmol) were combined in acetic acid (1.2 mL) and heated to 50° C. for 20 hours. The reaction was concentrated and methanol (2 ml) and 38% hydrochloric acid (0.3 ml) were added and the resulting mixture was stirred at 50° C. for 4 hours. The mixture was neutralized with saturated sodium bicarbonate and extracted with ethyl acetate, dried over magnesium sulfate, filtered and concentrated. Crude was purified on silica gel, eluting with a gradient from 0 to 7% methanol in ethyl acetate to give the target compound (3K) (3 mg, 12%) as a white solid.

1H NMR (500 MHz, CHLOROFORM-d) δ ppm 1.81 (s, 6H) 3.08 (t, 2H) 3.38 (s, 3H) 3.96 (t, 2H) 3.97 (s, 3H) 5.60 (s, 1H) 7.22 (d, 2H) 7.32 (d, 2H) 7.84 (s, 1H) 8.60 (s, 1H)

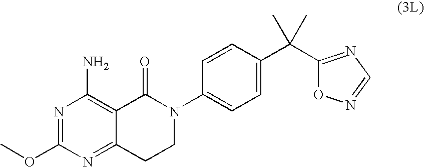

### 4-amino-2-methoxy-6-{4-[1-methyl-1-(1,2,4-oxadiazol-5-Methyl]phenyl}-7,8-dihydropyrido[4,3-d]pyrimidin-5(6H)-one

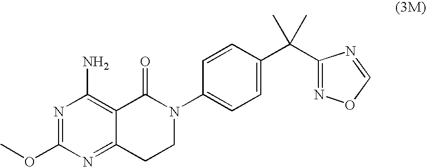

### 4-amino-2-methoxy-6-{4-[1-methyl-1-(1,2,4-oxadiazol-3-yl)ethyl]phenyl}-7,8-dihydropyrido[4,3-d]pyrimidin-5(6H)-one

(3J) (30 mg, 0.064 mmol), hydroxylamine hydrochloride (36 mg, 0.52 mmol) and triethylamine (0.11 mL, 0.77 mmol) were combined in acetic acid (1.2 mL) and heated to 50° C. for 3 hours. The reaction was concentrated and methanol (2 ml) and 38% hydrochloric acid (0.3 ml) were added and the resulting mixture was stirred at 50° C. for 4 hours. The mixture was neutralized with saturated sodium bicarbonate and extracted with ethyl acetate, dried over magnesium sulfate, filtered and concentrated. Crude was purified on preparative reverse phase HPLC

Preparative LC/MS method conditions:

MS mode: MS:APCI+scan range 200-900 daltons

Column: Phenomenex Luna (2) C18 21.2×150 mm, 5 um

Modifier: Formic Acid 0.1%

Method: 95% H20/5% MeCN (initial conditions) linear gradient to 5% H20/95% MeCN for 10.0 min,

then HOLD 0% H20/100% MeCN for 1.0 min. Flow rate, 28 mL/min.

QC Analysis method conditions:

MS mode: MS:APCI+scan range 200-900 daltons

Column: Phenomenex Luna (2) C18 4.6×150 mm, 5 um

Modifier: Formic Acid 0.1%

Method: 95% H20/5% MeCN (initial conditions)

linear gradient to 5% H20/95% MeCN for 10.0 min, then

HOLD 0% H20/100% MeCN for 1.0 min. Flow rate, 1.5 mL/min

(3L) (Peak 1) (2.5 mg, 10%)

1H NMR (500 MHz, CHLOROFORM-d) δ ppm 1.63 (s, 6H) 3.00 (t, 2H) 3.94 (t, 2H) 3.98 (s, 3H) 5.68 (s, 1H) 7.28 (s, 1H) 7.32-7.41 (m, 4H) 8.39 (s, 1H)

(3M) (Peak 2) (5.1 mg, 21%)

1H NMR (500 MHz, CHLOROFORM-d) δ ppm 1.89 (s, 6H) 3.07 (t, 2H) 3.94 (t, 2H) 3.98 (s, 3H) 5.55 (s, 1H) 7.31 (d, 2H) 7.38 (d, 2H) 8.37 (s, 1H) 8.61 (s, 1H)

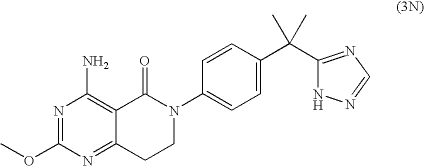

### 4-amino-2-methoxy-6-{4-[1-methyl-1-(1H-1,2,4-triazol-5-yOethyl]phenyl}-7,8-dihydropyrido[4,3-d]pyrimidin-5(6H)-one

(3J) (30 mg, 0.064 mmol) and hydrazine (0.02 mL, 0.640 mmol) were combined in acetic acid (1.2 mL) and heated to 50° C. for 3 hours. The reaction was concentrated and methanol (2 ml) and 38% hydrochloric acid (0.3 ml) were added and the resulting mixture was stirred at 50° C. for 4 hours. The mixture was neutralized with saturated sodium bicarbonate and extracted with ethyl acetate, dried over magnesium sulfate, filtered and concentrated. Crude was purified on silica gel eluting a gradient from 0 to 7% methanol in ethyl acetate to give the target compound (3N) (4.7 mg, 19%).

1H NMR (500 MHz, CHLOROFORM-d) δ ppm 1.82 (s, 6H) 3.03 (t, 2H) 3.92 (t, 2H) 3.97 (s, 3H) 5.76 (s, 1H) 7.26 (d, 2H) 7.35 (d, 2H) 7.98 (s, 1H) 8.50 (s, 1H)

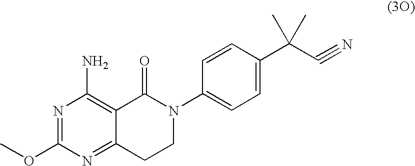

### 2-[4-(4-amino-2-methoxy-5-oxo-7,8-dihydropyrido[4,3-d]pyrimidin-6(5H)-yl)phenyl]-2-methylpropanenitrile

A solution of 4-Bromophenylacetonitrile (10.0 g, 51.0 mmol) in tetrahydrofuran (200 mL) was added slowly to a suspension of sodium hydride (60% in mineral oil, 6.0 g, 153 mmol) in tetrahydrofuran (400 mL) at room temperature over 30 minutes. After complete addition, methyl iodide (7.6 mL, 122 mmol) was added slowly over 30 minutes, maintaining the reaction temperature below 40° C. by occasional immersion into a water bath. The reaction was then stirred overnight at room temperature. The mixture was poured into water (500 mL) and extracted into ethyl acetate (2×300 mL). The combine organic layers were washed with brine (2×300 mL), dried over magnesium sulfate, filtered and concentrated.

Crude purified on silica gel eluting with 2% ethyl acetate in hexanes) to give 2-(4-bromophenyl)-2-methylpropanenitrile (11 g, 96%) as a yellow oil.

1H NMR (300 MHz, CDCl3): δ ppm 1.70 (s, 6H), 7.35 (dd, 2H), 7.53 (dd, 2H).

To degassed toluene (200 mL) was added palladium acetate (0.30 g, 1.3 mmol) and x-phos (1.3 g, 2.7 mmol) and the mixture degassed a further 5 minutes. To the reaction mixture was then added cesium carbonate (35.0 g, 107 mmol), beta alanine ethyl ester hydrochloride (6.2 g, 40.4 mmol) and 2-(4-bromophenyl)-2-methylpropanenitrile (6.0 g, 26.8 mmol). The reaction mixture was heated at reflux for 2 hours, cooled and filtered through celite. Filtrate concentrated and the residue further purified on silica eluting with 25% ethyl acetate in heptane to give ethyl 3-(4-(2-cyanopropan-2-yl)phenylamino)propanoate (4.05 g, 58%) as a yellow solid.

1H NMR (300 MHz, CDCl3): δ ppm 1.26 (t, 3H), 1.66 (s, 6H), 2.60 (t, 2H), 3.44 (t, 2H), 4.15 (q, 2H), 4.18 (br s, 1H), 6.61 (d, 2H), 7.26 (d, 2H).

To a suspension of cyanoacetic acid (1.990 g, 23.4 mmol) and dimethylformamide (0.05 mL) in dichloromethane (30 mL) was added oxalyl chloride (2.03 mL, 23.4 mmol) and the mixture stirred for 90 minutes. To this was then added ethyl 3-(4-(2-cyanopropan-2-yl)phenylamino)propanoate (4.05 g, 15.6 mmol) and the reaction cooled to 0° C. To the reaction was then added triethylamine (7.6 g, 75 mmol), stirred for 1 hour and then warmed to room temperature and stirred for 18 hours. Reaction mixture was washed with saturated aqueous sodium sulfate (50 mL) and organics dried over magnesium sulfate and concentrated to give ethyl 3-(2-cyano-N-(4-(2-cyanopropan-2-yl)phenyl)acetamido)propanoate (3.95 g (77%).

1H NMR (300 MHz, CDCl3): δ ppm 1.20 (t, 3H), 1.70 (s, 6H), 2.57 (t, 2H), 3.18 (s, 2H), 4.0 (t, 2H), 4.09 (q, 2H), 7.26 (d, 2H), 7.58 (d, 2H).

A mixture of ethyl 3-(2-cyano-N-(4-(2-cyanopropan-2-yl)phenyl)acetamido)propanoate (3.95 g, 12.1 mmol), 1,8-diazabicycloundec-7-ene (2.22 g, 14.5 mmol) and methanol (50 mL) was heated at reflux for 2.5 hours. The reaction was then cooled, concentrated and dissolved in water (75 mL). 2 M aqueous hydrochloric acid (15 mL) was added drop wise to form a precipitate. The mixture was then extracted into ethyl acetate (2×100 mL) and the combined organic phases were washed with brine, dried over magnesium sulfate and concentrated to 30 mL. The resulting yellow precipitate was filtered, washed with hexanes and dried to give 1-(4-(2-cyanopropan-2-yl)phenyl)-4-hydroxy-2-oxo-1,2,5,6-tetrahydropyridine-3-carbonitrile (2.83 g, 83%).

1H NMR (300 MHz, CD3OD): δ ppm 1.71 (s, 6H), 2.87 (t, 2H), 3.87 (t, 2H), 7.35 (d, 2H), 7.53 (d, 2H).

To a suspension of 1-(4-(2-cyanopropan-2-yl)phenyl)-4-hydroxy-2-oxo-1,2,5,6-tetrahydropyridine-3-carbonitrile (2.80 g, 9.95 mmol) and dimethylformamide (0.05 mL) in dichloromethane (50 mL) was added oxalyl chloride (2.78 mL, 31.8 mmol) and the mixture stirred for 90 minutes. Reaction concentrated and toluene added and concentrated to dryness. Methanol (80 mL) was added and reaction mixture was heated to reflux for 4 hours. Reaction was cooled to room temperature and concentrated. The residue was triturated with methanol to give (1-(4-(2-cyanopropan-2-yl)phenyl)-4-methoxy-2-oxo-1,2,5,6-tetrahydropyridine-3-carbonitrile) (2.25 g, 76%).

1H NMR (300 MHz, CDCl3): δ ppm 1.71 (s, 6H), 2.85 (t, 2H), 3.84 (t, 2H), 4.18 (s, 3H), 7.31 (d, 2H), 7.47 (d, 2H).

A mixture of 1-(4-(2-cyanopropan-2-yl)phenyl)-4-methoxy-2-oxo-1,2,5,6-tetrahydropyridine-3-carbonitrile (2.25 g, 7.61 mmol), 1,8-diazabicycloundec-7-ene (1.74 mL, 11.4 mmol), O- methylisourea (2.68 g, 24.3 mmol) and methanol (75 mL) was heated at reflux for 2 hours. The reaction cooled and concentrated. The residue dissolved in ethyl acetate (70 mL), washed with brine (2×50 mL), dried over magnesium sulfate and concentrated. The residue was re-crystallized from ethyl acetate/ethanol to give the title compound (30) (1.22 g, 47%) as a white solid.

1H NMR (300 MHz, CDCl3): δ ppm 8.57 (bs, 1H), 7.53 (d, J=8.8, 2H), 7.35 (d, J=8.8, 2H), 5.56 (bs, 1H), 3.96 (s, 3H), 3.95 (t, J=6.8, 2H), 3.06 (t, J=6.8, 2H), 1.73 (s, 6H).

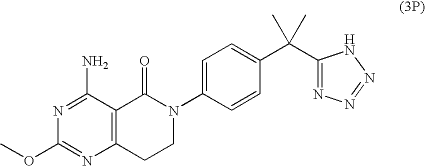

### 4-amino-2-methoxy-6-{4-[1-methyl-1-(1H-tetrazol-5-yl)ethyl]phenyl}-7,8-dihydropyrido[4,3-d]pyrimidin-5(6H)-one

A mixture of (30) (480 mg, 1.42 mmol), sodium azide (930 mg, 14.3 mmol), ammonium chloride (802 mg, 15.0 mmol) and dimethylformamide (12 mL) were stirred at 130° C. for 30 hours. Water (80 mL) was added and the mixture was extracted into ethyl acetate (3×50 mL), washed with brine (100 mL), dried over magnesium sulfate and concentrated. Crude purified on silica gel eluting with 1% acetic acid in 10% methanol in ethyl acetate. Isolated solid was recrystallized from ethanol to give the title compound (3P) (64 mg) as a white solid.

1H NMR (300 MHz, CD3OD): δ ppm 7.31-7.27 (m, 4H), 3.92 (s, 3H), 3.91 (t, J=6.6, 2H), 3.00 (t, J=6.6, 2H), 1.84 (s, 6H).

m/z (M+1)=380.9

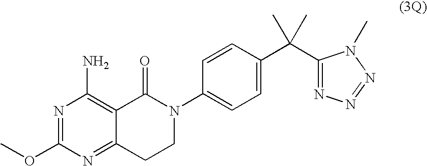

### 4-amino-2-methoxy-6-{4-[1-methyl-1-(1-methyl-1H-tetrazol-5-yl)ethyl]phenyl}-7,8-dihydropyrido[4,3-d]pyrimidin-5(6H)-one

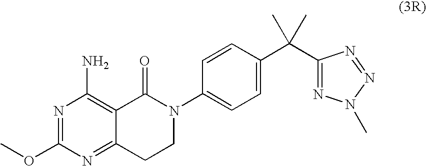

### 4-amino-2-methoxy-6-{4-[1-methyl-1-(2-methyl-2H-tetrazol-5-yl)ethyl]phenyl}-7,8-dihydropyrido[4,3-d]pyrimidin-5(6H)-one

To a suspension of (3P) (300 mg, 0.79 mmol) in acetonitrile (10 mL) was added iodomethane (98 uL, 1.6 mmol), followed by triethylamine (330 uL, 2.4 mmol) and the resulting solution was stirred at ambient temperature for 60 hours then heated to 40° C. for 16 hours. An additional 98 uL (1.6 mmol) of iodomethane was then added and the reaction mixture stirred at 40° C. for 3 hours. Reaction cooled to room temperature and concentrated. The crude product was then purified by preparative HPLC to give (3R) (53 mg) and (3Q) (8 mg).

The assignment of the two isomers of this product is tentatively made based on sterics.

(3Q) 1H NMR (400 MHz, (CH3)2SO): δ ppm 8.31 (br. s, 1H), 7.76 (br. s, 1H), 7.32 (s, 2H), 7.14 (s, 2H), 3.86-3.81 (m, 5H), 3.49 (s, 3H), 2.91 (s, 2H), 1.75 (s, 6H) ppm.

(3R) 1H NMR (400 MHz, (CH3)2SO): δ ppm 8.30 (br. s, 1H), 7.74 (br. s, 1H), 7.23 (s, 4H), 7.29 (s, 3H), 3.83 (t, J=6.9 Hz, 2H), 3.81 (s, 3H), 2.90 (t, J=6.9 Hz, 2H), 1.74 (s, 6H) ppm.

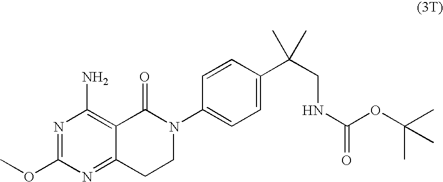

### tert-butyl {2-[4-(4-amino-2-methoxy-5-oxo-7,8-dihydropyrido[4,3-d]pyrimidin-6(5H)-yl)phenyl]-2-methylpropyl}carbamate

Commercial activated Raney nickel (50% in water, 500 mg) was washed with 2M sodium hydroxide (2×10 mL), followed by water (3×10 mL), then methanol (3×10 mL). The nickel was then rinsed into a parr hydrogenator (300 mL volume). A solution of (30) (500 mg, 1.48 mmol) in 50 mL of 20% ammonia in methanol was then added. The reactor was charged with hydrogen to 50 bar and stirred at room temperature for 16 hours then stirred at 30° C. for 5 hours at 50 bar pressure. As the reaction was still not complete, a further 500 mg of Raney nickel (washed as above) was added and the reaction stirred at room temperature for 48 hours. After discharging the pressure, the mixture was filtered through a pad of celite and the filter cake washed with methanol. The combined filtrates were concentrated to give 4-amino-6-(4-(1-amino-2-methylpropan-2-yl)phenyl)-2-methoxy-7,8-dihydropyrido[4,3-d]pyrimidin-5(6H)-one (3S) which was used crude in next step.

(3S) (145 mg, 0.42 mmol) was dissolved in dichloromethane (5 mL) and di-tert-butyl dicarbonate (102 mg, 0.47 mmol) was added. The resulting solution was then stirred at room temperature for 16 hours. Reaction concentrated and the crude product purified by flash chromatography eluting with 10% methanol in ethyl acetate to give a viscous, colorless oil that was triturated with methyl tert-butyl ether to give the target compound, (3T) (81 mg, 44%) as a colorless solid.

1H NMR (400 MHz, CDCl3): δ ppm 8.60 (br. s, 1H), 7.40 (d, J=8.3 Hz, 2H), 7.28 (d, J=8.7 Hz, 2H), 5.51 (br. s, 1H), 4.34 (br. s, 1H), 3.95 (s, 3H), 3.94 (t, J=3.9 Hz, 2H), 3.33 (d, J=6.4 Hz, 2H), 3.05 (t, J=6.9 Hz, 2H), 1.40 (s, 9H), 1.32 (s, 6H) ppm.

m/z (M+1)=442.1

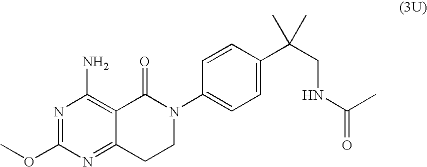

### N-{2-[4-(4-amino-2-methoxy-5-oxo-7,8-dihydropyrido[4,3-d]pyrimidin-6(5H)-yl)phenyl]-2-methylpropyl}acetamide

Acetyl chloride (114 mg, 1.45 mmol) was added slowly to a solution of (3S) (490 mg, 1.44 mmol) and trietylamine (353 mg, 3.45 mmol) in dichloromethane (20 mL) at 10° C. over 3 minutes. Reaction stirred at room temperature for 2 hours. Water (30 mL) was added and the mixture was stirred for 10 minutes. The organic layer was separated, dried over magnesium sulfate and concentrated. The residue was taken up in dichloromethane (20 ml) and isopropanol (15 mL) was added. Reaction mixture was concentrated until the volume was down to 10 mL and upon standing at room temperature for 30 minutes, solids crashed out of solution. Solids filtered off to give the target compound, (3U) (305 mg, 55%) as a white solid.

1H NMR (300 MHz, CDCl3): δ ppm 1.33 (s, 6H), 1.91 (s, 3H), 3.06 (t, 2H), 3.49 (d, 2H), 3.95 (d, 2H), 3.96 (s, 3H), 5.18 (br s, 1H), 5.53 (br s, 1H), 7.13 (d, 2H), 7.40 (d, 2H), 8.59 (br s, 1H).

m/z (M+1)=384.0

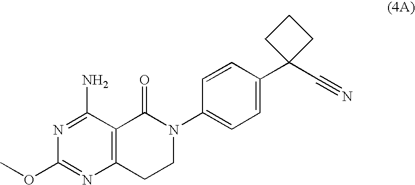

### 1-[4-(4-amino-2-methoxy-5-oxo-7,8-dihydropyrido[4,3-d]pyrimidin-6(5H)-yl)phenyl]cyclobutanecarbonitrile

Potassium nitrate (7.88 g, 77.0 mmol) was suspended in sulfuric acid (45 mL) at 0° C. and stirred for 30 minutes until a clear and colorless solution was obtained (NOTE—a blast shield is highly recommended). An addition funnel was charged with 1-phenylcyclobutanecarbonitrile (11.40 g, 72.5 mmol), and this neat starting material was added drop wise at such a rate that the internal reaction temperature did not exceed 10° C. Upon completion of the addition (which required 90 min), the mixture was poured onto 300 g of ice and stirred vigorously for 30 minutes. The resulting suspension was filtered, and the solid was washed with water and dried under vacuum to afford give 1-(4-nitrophenyl)cyclobutanecarbonitrile (13.53 g, 92%) as a light tan powder.

1H NMR (500 MHz, CHLOROFORM-d) δ ppm 2.11-2.21 (m, 1H) 2.47-2.58 (m, 1H) 2.66 (s, 2H) 2.88-2.96 (m, 2H) 7.63 (d, J=8.54 Hz, 2H) 8.29 (d, J=8.54 Hz, 2H).

A steel hydrogenation vessel was loaded with 1-(4-nitrophenyl)cyclobutanecarbonitrile (103.6 g, 0.51 mol), 10% palladium on activated carbon (10.3 g; contains ˜50% of water), and 2-methyltetrahydrofuran (1.3 L). The mixture was stirred under 30 psi of hydrogen gas at 45° C. for 4 h. The mixture was filtered through a pad of celite and filtrate concentrated. Heptane (1 L) was added to the obtained oil and the heterogeneous mixture was stirred while slowly cooled to room temperature, causing the product aniline to solidify. The solid was filtered off and dried in vacuum to give 1-(4-aminophenyl)cyclobutanecarbonitrile (86.6 g, 98%).

1H NMR (CHLOROFORM-d) δ ppm 7.12-7.25 (m, 2H), 6.61-6.76 (m, 2H), 3.68 (br. s., 2H), 2.68-2.88 (m, 2H), 2.48-2.64 (m, 2H), 2.30-2.45 (m, 1H), 1.94-2.14 (m, 1H)

A mixture of 1-(4-aminophenyl)cyclobutanecarbonitrile (42.2 g, 245 mmol), triethylamine (27.1 mL, 394 mmol), and ethyl acrylate (28.0 mL, 258 mmol) were combined in ethanol (27 mL) and heated to reflux for 24 hours. The mixture was concentrated to dryness and toluene (600 mL) added and concentrated to dryness to give ethyl N-[4-(1-cyanocyclobutyl)phenyl]beta-alaninate as brown oil, which was used without further purification.

1H NMR (CHLOROFORM-d) δ ppm 7.22 (d, 2H), 6.63 (d, 2H), 4.12-4.21 (m, 3H), 3.47 (q, J=6.3 Hz, 2H), 2.74-2.83 (m, 2H), 2.53-2.66 (m, 4H), 2.33-2.45 (m, 1H), 2.00-2.11 (m, 1H), 1.28 (t, 3H)

Ethyl N-[4-(1-cyanocyclobutyl)phenyl]-beta-alaninate was combined with cyanoacetic acid (22.9 g, 270 mmol) and 4-dimethylaminopyridine (2.30 g, 18.8 mmol) in N,N-dimethylformamide (400 mL) and cooled to 0° C. Diisopropylcarbodiimide (41.7 mL, 270 mmol) was then added drop wise over 30 minutes. Once addition was complete, the reaction was slowly warmed up to room temperature and stirred for 16 hours. Reaction was then poured into saturated aqueous sodium bicarbonate (600 mL) and stirred for 30 mintues. Ethyl acetate (1 L) was added and the mixture was filtered to remove the insoluble diisopropylurea. The phases of the filtrate were separated, and the organic phase was washed with brine and dried over sodium sulfate and concentrated to give ethyl N-(cyanoacetyl)-N-[4-(1-cyanocyclobutyl)phenyl]-beta-alaninate as yellow oil that was used with out further purification in the following step.

ethyl N-(cyanoacetyl)-N-[4-(1-cyanocyclobutyl)phenyl]-beta-alaninate and 1,8-diazabicyclo[5.4.0]undec-7-ene (350 mmol) were combined in methanol (400 mL) and heated to 70° C. for 30 minutes. The mixture was concentrated to dryness then partitioned between water (400 mL) and 2:1 ethyl acetate:heptane (400 mL). The aqueous phase was separated and acidified to pH 2 by the addition of 1M hydrochloric acid (400 mL). The precipitate was filtered off and washed with water (300 mL) and 2:1 ethyl acetate:heptane (300 mL) give 1-(4-(1-cyanocyclobutyl)phenyl)-4-hydroxy-2-oxo-1,2,5,6-tetrahydropyridine-3-carbonitrile (31.7 g, 44% over 3 steps) as an off-white solid.

1H NMR (DMSO-d6) δ ppm 7.39-7.45 (m, 2H), 7.31 (d, 2H), 3.78 (t, J=6.7 Hz, 2H), 2.79 (t, 2H), 2.66-2.75 (m, 2H), 2.53-2.64 (m, 2H), 2.16-2.31 (m, 1H), 1.91-2.04 (m, 1H)

m/z (M+1)=294.4

1-(4-(1-Cyanocyclobutyl)phenyl)-4-hydroxy-2-oxo-1,2,5,6-tetrahydropyridine-3-carbonitrile (50.0 g, 170 mmol) and N,N-dimethylformamide (0.66 mL, 8.5 mmol) in dichloromethane (350 mL) was cooled to 0° C. Oxalyl chloride (18.0 mL, 203 mmol) was added over 15 minutes. The mixture was warmed to room temperature over 2 hours. Methanol (300 mL) was then added as a steady stream, and the mixture was heated at 45° C. for 16 hours. The mixture was cooled to room temperature and concentrated to get rid of most of the dichloromethane. Methanol (200 mL) was added and the thick slurry was stirred for 2 hours. The solid was filtered and dried under vacuum to give 1-(4-(1-cyanocyclobutyl)phenyl)-4-methoxy-2-oxo-1,2,5,6-tetrahydropyridine-3-carbonitrile (48.3 g, 92%) as an off-white powder.

1H NMR (400 MHz, DMSO-d6) δ ppm 1.91-2.03 (m, 1H) 2.18-2.31 (m, 1H) 2.54-2.63 (m, 2H) 2.67-2.75 (m, 2H) 3.03 (t, J=6.73 Hz, 2H) 3.85 (t, J=6.73 Hz, 2H) 4.01 (s, 3H) 7.33 (d, J=8.78 Hz, 2H) 7.44 (d, J=8.78 Hz, 2H)

m/z (M+1)=308.4

1-(4-(1-Cyanocyclobutyl)phenyl)-4-methoxy-2-oxo-1,2,5,6-tetrahydropyridine-3-carbonitrile (12.04 g, 37.9 mmol) and cyanamide (1.64 g, 41.0 mmol) were suspended in methanol (200 mL) at room temperature. A solution of 25% sodium methoxide in methanol (45.0 mmol) was then added drop wise over 10 minutes to obtain a clear homogeneous solution of the intermediate cyanamide adduct. In one portion, sulfuric acid (5.06 mL, 94.9 mmol) was added, and the mixture was heated to 50° C. for 16 hours. The mixture was then cooled to room temperature and basified to pH 10-11 by the addition of 1N sodium hydroxide, and the thick suspension was stirred for 20 minutes. The solid was filtered, washed with cold methanol and water, and dried under vacuum to obtain the crude product as a mixture contaminated with the vinylogous amide (4-amino-1-[4-(1-cyanocyclobutyl)phenyl]-2-oxo-1,2,5,6-tetrahydropyridine-3-carbonitrile). This solid mixture was heated to reflux in methanol (150 mL) for 3 hours then cooled to room temperature and filtered. The solid collected was then dissolved in a minimal amount of acetic acid (30 mL) at 60° C. to obtain a clear yellow solution. Water was then added drop wise at 60° C. until the cloudiness persisted, and the mixture was allowed to return to room temperature. Another 50 mL of water was added and the fine suspension was filtered, washed with water, and dried under vacuum to afford the title compound (4A) (6.80 g, 51%) as a light yellow solid.

¹H NMR (500 MHz, DMSO-d₆) δ ppm 1.97-2.06 (m, 1H) 2.23-2.34 (m, 1H) 2.59-2.67 (m, 2H) 2.71-2.79 (m, 2H) 2.96 (t, J=6.71 Hz, 2H) 3.86 (s, 3H) 3.91 (t, J=6.71 Hz, 2H) 7.39-7.44 (d, J=8.54, 2H) 7.47-7.51 (d, J=8.54, 2H) 7.81 (br. s., 1H) 8.35 (br. s., 1H).

m/z (M+1)=350.4

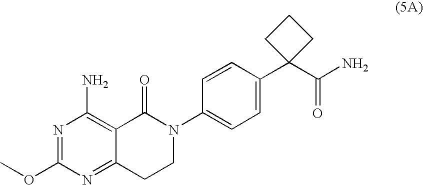

### 1-[4-(4-amino-2-methoxy-5-oxo-7,8-dihydropyrido[4,3-d]pyrimidin-6(5H)-yl)phenyl]cyclobutanecarboxamide

Sodium hydride (3.5 g, 88 mmol) was stirred as a suspension in dimethylformamide (250 ml) under argon. This was warmed to 35° C. and methyl 2-(4-bromophenyl)acetate (10 g, 44 mmol) in dimethylformamide (100 mL) was added drop wise over 1 hour and then stirred at 30° C. for 1 hour. To this the 1,3-dibromopropane (4.4 ml, 44 mmol) in dimtheylformamide (50 ml) was added drop wise over 1 hour, and this was left to stir at room temperature overnight. The reaction was incomplete. Sodium hydride (3.5 g, 88 mmol) was prepared in dimethylformamide (100 ml) at 35° C. and was added to this drop wise to the reaction mixture over 1 hour. This was again left to stir at room temperature overnight. Saturated aqueous ammonium chloride solution (200 ml) was carefully added, followed by water (500 ml). The product was extracted with ethyl acetate (2×500 ml), washed with water (3×500 ml), and brine (2×500 ml). The organic solution was then dried over magnesium sulfate, filtered, and evaporated. The crude product was purified by flash chromatography (12.5% ethyl acetate in heptane)to methyl 1-(4-bromophenyl)cyclobutanecarboxylate (900 mg, 3.3 mmol, 7.5%).

1H NMR (400 MHz CDCl3) δ ppm 7.45 (d, 2H), 7.15 (d, 2H), 3.65 (s, 3H), 2.80 (m, 2H), 2.45 (m, 2H), 2.05 (m 1H), 1.85 (m, 1H)

To degassed toluene (25 mL) was added palladium acetate (18 mg, 0.0825 mmol) and Xphos (79 mg, 0.165 mmol) and the mixture was degassed a further 30 minutes. To this reaction mixture was added methyl 1-(4-bromophenyl)cyclobutanecarboxylate (900 mg, 3.3 mmol) and cesium carbonate (3.2 g, 9.9 mmol) and this was degassed for a further 10 minutes. Beta-alanine ethyl ester (920 mg, 6.6 mmol) and diisopropylethyl amine (1.1 ml, 6.6 mmol) were added and this was brought to reflux and stirred overnight. The reaction was cooled to room temperature and poured onto water (100 ml). It was then extracted with ethyl acetate (2×100 ml), dried over magnesium sulfate, filtered, and evaporated to give methyl 1-(4-(3-ethoxy-3-oxopropylamino)phenyl)cyclobutanecarboxylate. Taken on crude to the next reaction.

1H NMR (400 MHz, CDCl3) 7.1 (d, 2H), 6.55 (d, 2H), 4.15 (m, 2H), 3.65 (s, 3H), 3.45 (m, 2H), 2.75 (m, 2H), 2.60 (t, 2H), 2.45 (m, 2H), 1.95 (m, 1H), 1.85 (m, 1H), 1.35 (m, 3H)

Methyl 1-(4-(3-ethoxy-3-oxopropylamino)phenyl)cyclobutanecarboxylate (crude, 3.3 mmol) was dissolved in dichloromethane (50 ml) and 3-[cyano(ethyl)amino]propyl-dimethylazanium chloride (883 mg, 1.4 mmol), cyanoacetic acid (561 mg, 6.6 mmol), and 4-dimethylaminopyridine (403 mg, 3.3 mmol) were added. This was then left to stir at room temperature over 4 days. The solution was washed with 1M aqueous hydrochloric acid (50 ml), extracted with dichloromethane (2×50 ml), dried over magnesium sulfate, filtered, and evaporated. It was purified by flash chromatography eluting with 50% ethyl acetate in heptane to give methyl 1-(4-(2-cyano-N-(3-ethoxy-3-oxopropyl)acetamido)phenyl)cyclobutanecarboxylate (700 mg, 1.88 mmol, 57% over 2 steps).

1H NMR (400 MHz, CDCl3) δ ppm 7.40 (d, 2H), 7.15 (d, 2H), 4.0 (q, 2H), 3.65 (s, 3H), 3.2 (s, 2H), 2.85 (m, 2H), 2.55 (m, 2H), 2.45 (m, 2H), 2.10 (m, 1H), 1.90 (m, 1H), 1.35 (m, t)

Methyl 1-(4-(2-cyano-N-(3-ethoxy-3-oxopropyl)acetamido)phenyl)cyclobutanecarboxylate (700 mg, 1.88 mmol) was stirred in methanol (10 ml) with 1,8-diazabicycloundec-7-ene (0.336 ml, 2.26 mmol) at room temperature overnight. It was then evaporated to dryness, and stirred in 1M aqueous hydrochloric acid (30 ml) for 15 minutes. It was then filtered and dried to give methyl 1-(4-(3-cyano-4-hydroxy-2-oxo-5,6-dihydropyridin-1(2H)-yl)phenyl)cyclobutanecarboxylatea (380 mg, 1.16 mmol, 62%) as a white solid.

1H NMR (400 MHz, DMSO-d6) δ ppm 7.20 (s, 4H), 3.75 (t, 2H), 3.55 (s, 3H), 2.75 (t, 2H), 2.70 (m, 2H), 2.40 (m, 2H), 1.90 (m, 1H), 1.75 (m, 1H)

Methyl 1-(4-(3-cyano-4-hydroxy-2-oxo-5,6-dihydropyridin-1(2H)-yl)phenyl)cyclobutanecarboxylate (380 mg, 1.16 mmol) was dissolved in dichloromethane (10 ml) and oxalyl chloride (0.304 ml, 3.48 mmol) was added. To this 2 drops of dimethylformamide were added and the suspension was left to stir at room temperature for 3 hours. After this time all the solid dissolved. This was then evaporated to dryness, toluene (50 ml) added, and again evaporated to dryness. The residue was taken up in methanol (50 ml) and this was left to reflux overnight. The solution was cooled to room temperature, and then further in an ice bath. Solids crashed out and were filtered off to give methyl 1-(4-(3-cyano-4-methoxy-2-oxo-5,6-dihydropyridin-1(2H)-yl)phenyl)cyclobutanecarboxylate. Filtrate was concentrated and ethyl acetate (10 ml) added. Solids filtered off and combined with first batch (390 mg, 0.97 mmol, 84%).

1H NMR (300 MHz DMSO-d6) δ ppm 7.25 (s, 4H), 4.0 (s, 3H), 3.80 (t, 2H), 3.55 (s, 3H), 3.05 (t, 2H), 2.70 (m, 2H), 2.40 (m, 2H), 1.90 (m, 1H), 1.75 (m, 1H)

Methyl 1-(4-(3-cyano-4-methoxy-2-oxo-5,6-dihydropyridin-1(2H)-yl)phenyl)cyclobutanecarboxylate was added to a solution containing cyanamide (116 mg, 2.75 mmol) and sodium methoxide (178 mg, 3.3 mmol) in methanol (20 ml). This was stirred at room temperature for 3 hours and concentrated sulfuric acid (275 ul, 5.5 mmol) was added. This was then left to reflux overnight. The reaction was cooled to room temperature, and evaporated to dryness. Aqueous sodium hydrogen carbonate solution (10 ml) and ethyl acetate (10 ml) were added and the mixture filtered to give methyl 1-(4-(4-amino-2-methoxy-5-oxo-7,8-dihydropyrido[4,3-d]pyrimidin-6(5H)-yl)phenyl)cyclobutanecarboxylate (280 mg, 0.74 mmol, 67%) as a cream solid.

1H NMR (400 MHz, DMSO-d6) δ ppm 8.35 (s, 1H), 7.70 (s, 1H), 7.25 (m, 4H), 3.85 (m, 2H), 3.80 (s, 3H), 3.55 (s, 3H), 2.90 (m, 2H), 2.65 (m, 2H), 2.40 (m, 2H), 1.90 (m, 2H)

Methyl 1-(4-(4-amino-2-methoxy-5-oxo-7,8-dihydropyrido[4,3-d]pyrimidin-6(5H)-yl)phenyl)cyclobutanecarboxylate (280 mg, 0.74 mmol) was dissolved in methanol (7 ml), dioxane (2 ml), and water (2 ml). This was warmed to 40° C. and dioxane (5 ml) was added. Lithium hydroxide (342 mg, 8.14 mmol) was added and this was left to stir at 40° C. overnight. The reaction solution was cooled and evaporated until 2 ml remained. Water (10 ml) was added and the pH was adjusted to 4-5. A white solid was filtered off, washed with isopropanol (1 ml) and hexane (1 ml). The aqueous solution was extracted with ethyl acetate (2×30 ml), dried over magnesium sulfate, filtered, and evaporated to give a second crop of product. The two crops were combined and dried to give 1-(4-(4-amino-2-methoxy-5-oxo-7,8-dihydropyrido[4,3-d]pyrimidin-6(5H)-yl)phenyl)cyclobutanecarboxylic acid (130 mg, 0.35 mmol, 47%) as a white solid.

1H NMR (400 MHz, DMSO-d6) δ ppm 8.35 (s, 1H), 7.75 (s, 1H), 7.25 (m, 4H), 3.8 (m, 5H), 2.90 (m, 2H), 2.65 (m, 2H), 2.30 (m, 2H), 1.90 (m, 1H), 1.75 (m, 1H)

1-(4-(4-amino-2-methoxy-5-oxo-7,8-dihydropyrido[4,3-d]pyrimidin-6(5H)-yl)phenyl)cyclobutanecarboxylic acid was dissolved in dimethylformamide (3 ml) and 2-(1H-benzotriazole-1-yl)-1,1,3,3-tetramethyluronium hexafluorophosphate (66 mg, 0.43 mmol) and 3-[cyano(ethyl)amino]propyl-dimethylazanium chloride (67 mg, 0.35 mmol) were added. This was stirred at room temperature for 2 hours, and concentrated ammonia (1 ml) was then added. This was left to stir at room temperature overnight. A white solid had precipitated, which was filtered off and washed with isopropanol (0.5 ml), and hexane (5 ml) to give the target compound, (5A) (10 mg).

1H NMR (400 MHz, DMSO-d6) δ ppm 8.30 (s, 1H), 7.75 (s, 1H), 7.30 (d, 2H), 7.25 (d, 2H), 7.15 (s, 1H), 6.85 (s, 1H), 3.85 (m, 5H), 2.90 (t, 2H), 2.65 (m, 2H), 2.25 (m, 2H), 1.75 (m, 2H)

m/z (M+1)=368.0

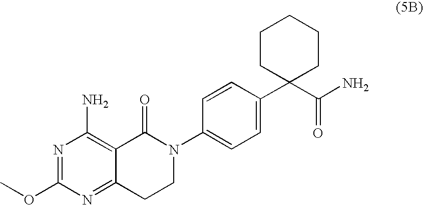

### 1-[4-(4-amino-2-methoxy-5-oxo-7,8-dihydropyrido[4,3-d]pyrimidin-6(5H)-yl)phenyl]cyclohexanecarboxamide

Prepared analogous to (5A) from methyl 1-(4-bromophenyl)cyclohexanecarboxylate which was prepared as follows:

Sodium hydride (12 g, 52 mmol) was suspended in tetrahydrofuran (200 mL) under argon and warmed to 35° C. Methyl 2-(4-bromophenyl)acetate (26 mmol) in tetrahydrofuran added drop wise to reaction over 1 hour. The reaction mixture was then kept at this temperature for 1 hour until all gas evolution has ceased. The 1,5-diiodopentane (17 g, 52 mmol) was then added drop wise as a solution in tetrahydrofuran (100 mL) and the reaction mixture stirred at 35° C. for a further hour and at ambient temperature overnight. After this time, the reaction mixture was cooled to 0° C. and quenched by the addition of dry silica, filtered and the solvent removed under vacuum. The crude product was then purified by flash chromatography eluting with 33% ethyl acetate in heptane to give methyl 1-(4-bromophenyl)cyclohexanecarboxylate (15.3 g, 99% yield) as a yellow oil.

1H NMR (400 MHz, CDCl3): δ ppm 7.45-7.38 (m, 2H), 7.27-7.24 (m, 2H), 3.63 (s, 3H), 2.43 (d, J=13.3 Hz, 2H), 1.71-0.80 (m, 8H) ppm.

(5B): 1H NMR (400 MHz, SO(CH3)2): δ ppm 8.32 (br. s, 1H), 7.79 (br. s, 1H), 7.33 (d, 2H), 7.28 (d, 2H), 7.03 (bs, 1H), 6.78 (bs, 1H), 3.85 (t, 2H), 3.53 (s, 3H), 2.91 (t, 2H), 2.34 (br. d, 2H), 1.68-1.21 (m, 8H) ppm.

m/z (M+1)=396.1

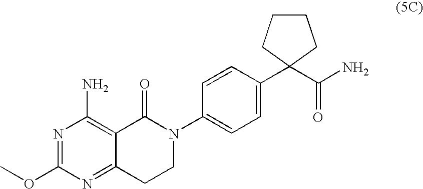

### 1-[4-(4-amino-2-methoxy-5-oxo-7,8-dihydropyrido[4,3-d]pyrimidin-6(5H)-yl)phenyl]cyclopentanecarboxamide

Prepared analogous to (5A) from methyl 1-(4-bromophenyl)cyclopentanecarboxylate which was prepared as follows:

Methyl 2-(4-bromophenyl)acetate (73.0 g, 0.32 mol) was dissolved in tetrahydrofuran (750 mL) and 1,4-diiodobutane (25.5 g, 0.64 mol) was added. The mixture was stirred under a flow of argon and sodium hydride (60% on oil, 100.0 g, 0.32 mol) was added slowly in portions. After the addition was complete, the mixture was stirred at room temperature for 16 hours. The mixture was poured onto ice-cold water (500 mL) and ethyl acetate was added (500 mL). The mixture was separated and the aqueous layer washed with ethyl acetate (500 mL). The organic layers were combined and washed with brine (1 L), dried over magnesium sulfate and concentrated to give methyl 1-(4-bromophenyl)cyclopentanecarboxylate (42.0 g, 47%) as a yellow solid.

1H NMR (CDCl3, 400 MHz): δ ppm 7.41 (d, 2H), 7.22 (d, 2H), 3.59 (s, 3H), 2.55-2.66 (m, 2H), 1.81-1.90 (m, 2H), 1.68-1.75 (m, 4H).

(5C): 1H NMR (DMSO, 400 MHz): δ ppm 8.32 (bs, 1H), 7.76 (bs, 1H), 7.33 (d, 2H), 7.23 (d, 2H), 7.03 (bs, 1H), 6.78 (bs, 1H), 3.78-3.89 (m, 5H), 2.90 (t, 2H), 2.50-2.67 (m, 2H), 1.50-1.73 (m, 6H).

m/z (M+1)=382.0

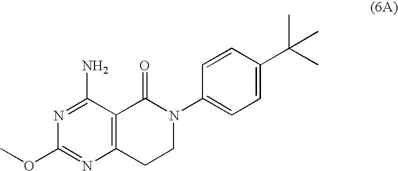

### 4-amino-6-(4-tert-butylphenyl)-2-methoxy-7,8-dihydropyrido[4,3-d]pyrimidin-5(6H)-one

To a stirred solution of 4-tert-butylphenol (2.88 g, 19.2 mmol) and triethylamine (4.01 ml, 28.8 mmol) in dichloromethane (101 mL) was added a solution triflic anhydride (6.8 g, 24 mmol) drop wise. The mixture was continued to stir at 0° C. for 2 hrs. The reaction mixture was washed with water and brine and dried over sodium sulfate, filtered and concentrated to give a dark brown oil. Product was purified on silica gel eluting with heptane to give 4-tert-butylphenyl trifluoromethanesulfonate (3.64 g 67.3%) as a clear oil.

1H NMR (400 MHz, CHLOROFORM-d) δ ppm 1.31 (s, 9H) 7.17 (d, J=8.72 Hz, 2H) 7.43 (d, J=8.72 Hz, 2H)

To degassed toluene (11.8 mL) was added beta alanine ethyl ester hydrochloride (0.354 g, 2.3 mmol), 4-tert-butylphenyl trifluoromethanesulfonate (0.5 g, 2 mmol), X-Phos (87 mg, 0.18 mmol), palladium acetate (42 mg, 0.186 mmol), diisopropylethyl amine (0.3 ml, 2 mmol), and cesium carbonate (1.73 g, 5.31 mmol). Reaction mixture was stirred at 110° C. for 20 hours. Reaction cooled to room temperature, diluted with water and extracted with ethyl acetate. Pooled organic layers washed with brine, dried over sodium sulfate and concentrated. Residue purified on silica eluting with 1% methanol in dichloromethane to give ethyl 3-(4-tert-butylphenylamino)propanoate (0.17 g , 40%) as an off-white solid.

1H NMR (400 MHz, CHLOROFORM-d) δ ppm 1.24 (s, 12H) 2.59 (t, J=6.44 Hz, 2H) 3.42 (t, J=6.44 Hz, 2H) 4.13 (q, J=7.06 Hz, 2H) 6.57 (d, J=8.72 Hz, 2H) 7.20 (d, J=8.72 Hz, 2 H)

A solution of ethyl 3-(4-tert-butylphenylamino)propanoate (170 mg, 0.682 mmol) and cyanoacetic acid (58.6 mg, 0.689 mmol) in 1.89 ml dimethylformamide was cooled to 0° C. Diisoproprylcarbodiimide (0.106 ml, 0.682) was added drop wise over 10 minutes. Once addition was complete, reaction was slowly warmed up to room temperature and stirred for 16 hours. Reaction mixture was diluted with 1:1 mixture of ethyl acetate:heptane and allowed to stand for 20 minutes. Solids filtered off and filtrate was washed with brine, dried over sodium sulfate, filtered and concentrated to give ethyl 3-(N-(4-tert-butylphenyl)-2-cyanoacetamido)propanoate (0.184 g, 85%) as an off-white product.

1H NMR (400 MHz, CHLOROFORM-d) δ ppm 1.19 (t, J=7.06 Hz, 3H) 1.32 (s, 9H) 2.57 (t, J=7.27 Hz, 2H) 3.18 (s, 2H) 3.87-4.09 (m, 4H) 7.11 (d, J=8.72 Hz, 2H) 7.45 (d, J=8.72 Hz, 2H)

m/z (M+1)=317.1

Ethyl 3-(N-(4-tert-butylphenyl)-2-cyanoacetamido)propanoate (184 mg, 0.582 mmol) and 1,5-Diazabicycloundecene (0.104 ml, 0.698 mmol) were dissolved in methanol (8.2 mL) and stirred at 80° for 18 hours. Reaction mixture was concentrated and 1 M hydrochloric acidl (2 mL) and water (10 mL) were added and stirred at room temperature for 16 hours. Solids filtered off and dried it in a vacuum oven at 50° C. to give 1-(4-tert-butylphenyl)-4-hydroxy-2-oxo-1,2,5,6-tetrahydropyridine-3-carbonitrile (0.131 g, 83%) as an off white solid.

1H NMR (400 MHz, DMSO-d6) δ ppm 1.25 (s, 9H) 2.78 (t, J=6.65 Hz, 2H) 3.74 (t, J=6.85 Hz, 2H) 7.15 (d, J=8.72 Hz, 2H) 7.35 (d, J=8.72 Hz, 2H)

m/z (M+1)=271.3

1-(4-tert-butylphenyl)-4-hydroxy-2-oxo-1,2,5,6-tetrahydropyridine-3-carbonitrile (131 mg, 0.485 mmol) in acetonitrile (4.85 mL) was cooled to 0° C. and 2.0 M solution of trimethylsilyl diazomethane in ether (0.28 mL, 1.79 mmol) was added over 5 minutes and slowly warmed up to room temperature for 30 minutes. Methanol (4 mL) and acetic acid (0.2 mL) were added and reaction was concentrated to give 1-(4-tert-butylphenyl)-4-methoxy-2-oxo-1,2,5,6-tetrahydropyridine-3-carbonitrile (6A-1) (0.141 g, >100%) as an orange solid that was used without further purification in the next step.

A solution of 1-(4-tert-butylphenyl)-4-methoxy-2-oxo-1,2,5,6-tetrahydropyridine-3-carbonitrile (141 mg, 0.496 mmol), O-methyl-isourea (118 mg, 1.59 mmol) and 1,5-diazabicyclo(5.4.0)undec-7-ene (0.371 ml, 2.48 mmol) in methanol (12.4 ml) was heated to 85° C. for 24 hours. Reaction was concentrated and the crude residue was purified by column chromatography eluting with 1:1-ethyl acetate:heptane to give the target compound, (6A) (31.7 mg, 19%) as a white solid.

1H NMR (400 MHz, DMSO-d6) δ ppm 1.26 (s, 9H) 2.91 (t, J=6.65 Hz, 2H) 3.81 (s, 3H) 3.84 (t, J=6.85 Hz, 2H) 7.23 (d, J=8.72 Hz, 2H) 7.38 (d, J=8.31 Hz, 2H)

m/z (M+1)=327.4

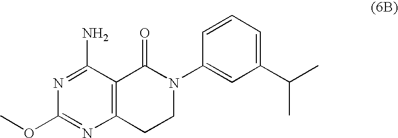

### 4-amino-6-(3-isopropylphenyl)-2-methoxy-7,8-dihydropyrido[4,3-d]pyrimidin-5(6H)-one

Prepared analogous to (6A) from the commercially available 3-isopropylphenol.

(6B): 1H NMR (400 MHz, CHLOROFORM-d) δ ppm 1.25 (d, J=7.06 Hz, 6H) 2.83-2.98 (m, 1H) 3.04 (t, J=6.65 Hz, 2H) 3.86-4.00 (m, 5H) 7.04-7.17 (m, 3H) 7.33 (t, J=7.89 Hz, 1H)

m/z (M+1)=313.5

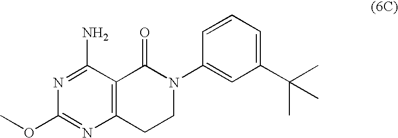

### 4-amino-6-(3-tert-butylphenyl)-2-methoxy-7,8-dihydropyrido[4,3-d]pyrimidin-5(6H)-one

Prepared analogous to (6A) from the commercially available 3-tertbutylphenol.

(6C): 1H NMR (400 MHz, CHLOROFORM-d) δ ppm 1.32 (s, 9H) 3.05 (dd, 2H) 3.88-3.98 (m, 5H) 7.11 (d, J=7.48 Hz, 1H) 7.26-7.39 (m, 3H)

m/z (M+1)=327.5

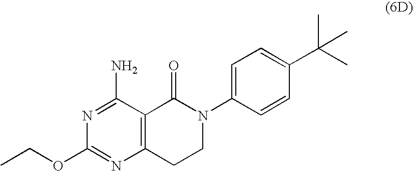

### 4-amino-6-(4-tert-butylphenyl)-2-ethoxy-7,8-dihydropyrido[4,3-d]pyrimidin-5(6H)-one

Prepared analogous to (6A) from the commercially available 4-tertbutylphenol. The final step (formation of the pyrimidine ring) is analogous to the exemplified procedure using O-ethyl-isourea.

(6D): 1H NMR (400 MHz, CHLOROFORM-d) δ ppm 1.31 (s, 9H) 1.38 (t, 3H) 3.03 (t, 2H) 3.91 (t, 2H) 4.36 (q, 2H) 5.56 (s, 1H) 7.21 (d, 2H) 7.42 (d, 2H) 8.63 (s, 1H)

m/z (M+1)=341.3

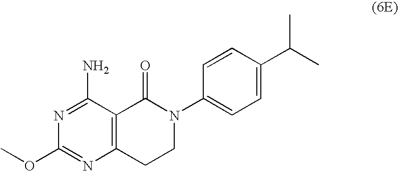

### 4-amino-6-(4-isopropylphenyl)-2-methoxy-7,8-dihydropyrido[4,3-d]pyrimidin-5(6-H)-one

Prepared analogous to (6A) from the commercially available 4-isopropylphenol.

(6E): 1H NMR (400 MHz, DMSO-d6) δ ppm 1.18 (d, J=7.06 Hz, 6H) 2.83-2.91 (m, 1H) 2.91 (t, J=6.65 Hz, 2H) 3.82 (s, 3H) 3.85 (t, 2H) 7.23 (d, J=2.08 Hz, 4H)

m/z (M+1)=313.4

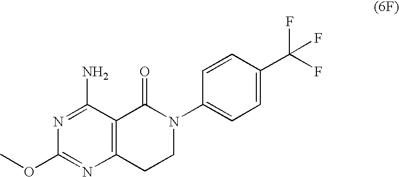

### 4-amino-2-methoxy-6-[4-(trifluoromethyl)phenyl]-7,8-dihydropyrido[4,3-d]pyrimidin-5(6H)-one

Prepared analogous to (6A) from the commercially available 4-trifluorophenol.

(6F): 1H NMR (500 MHz, DMSO-d6) δ ppm 2.98 (t, 2H) 3.86 (s, 3H) 3.98 (t, 2H) 7.59 (d, 2H) 7.77 (d, 2H) 7.88 (d, 1H) 8.31 (d, 1H)

m/z (M+1)=339.4

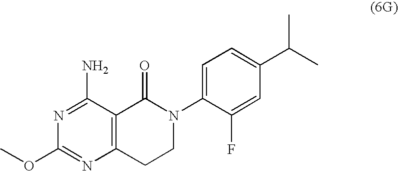

### 4-amino-6-(2-fluoro-4-isopropylphenyl)-2-methoxy-7,8-dihydropyrido[4,3-d]pyrimidin-5(6H)-one

Prepared analogous to (6A) from 2-fluoro-4-isopropylphenol which was prepared as follows:

3-Fluoro-4-hydroxyacetophenone (520 mg, 3.37 mmol) dissolved in tetrahydrofuran (20 mL) and cooled to −70° C. A 3.0M solution of methylmagnesium bromide in ether (1.17 mL, 10.1 mmol) was added drop wise to cold reaction mixture and stirred at 0° C. for 1 hour and then warmed up to room temperature and stirred for 18 hours. Saturated aqueous ammonium chloride (10 mL) and water (10 mL) were added and reaction mixture extracted with ethyl acetate (10 mL). Extract was washed with brine, dried over magnesium sulfate, filtered and concentrated give 2-fluoro-4-(2-hydroxypropan-2-yl)phenol (540 mg, 94%) which was used in the next reaction without further purification.

1H NMR (400 MHz, DMSO-d6) d ppm 1.33 (s, 6H) 4.89 (s, 1H) 6.78-6.83 (m, 1H) 6.99 (dd, 1H) 7.12 (dd, 1H) 9.50 (s, 1H)

To 2-fluoro-4-(2-hydroxypropan-2-yl)phenol (540 mg, 3.17 mmol) in acetic acid (30 mL) and 37% hydrochloric acid (0.5 mL) was added 10% palladium on carbon (100 mg). Reaction was stirred in a parr shaker at room temperature under hydrogen (40 PSI) for 18 hours. Reaction mixture was carefully filtered through a pad of celite under a steady stream of nitrogen. Filtrate concentrated and purified on silica gel, eluting with a gradient from 5% to 25% ethyl acetate in heptane to give 2-fluoro-4-isopropylphenol (489 mg, 65%).

1H NMR (500 MHz, CHLOROFORM-d) δ ppm 1.23 (d, 6H) 2.81-2.90 (m, 1H) 4.99 (s, 1H) 6.90 (dd, 1H) 6.92 (d, 1H) 6.94-6.97 (m, 1H)

(6G): 1H NMR (400 MHz, CHLOROFORM-d) δ ppm 1.24 (d, J=7.03 Hz, 6H) 2.86-2.95 (m, 1H) 3.05 (t, J=6.83 Hz, 2H) 3.85 (t, J=6.73 Hz, 2H) 3.94 (s, 3H) 5.50 (br. s., 1H) 6.99-7.08 (m, 2H) 7.21 (t, J=8.10 Hz, 1H) 8.56 (br. s., 1H)

m/z (M+1)=331.5

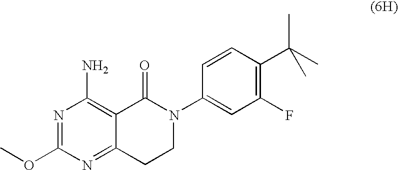

### 4-amino-6-(4-tert-butyl-3-fluorophenyl)-2-methoxy-7,8-dihydropyrido[4,3-d]pyrimidin-5(6H)-one

Prepared analogous to (6A) from the 4-tert-butyl-3-fluorophenol which was prepared as follows:

To a suspension of aluminum chloride (0.892 g, 6.69 mmol) in dichloromethane (13.4 mL) at 0° C. was added methyl t-butyl ether (0.797 ml, 6.69 mmol) and stirred for 30 minutes. 3-Fluorophenol (0.4 ml, 4 mmol) in dichloromethane was added and the mixture was stirred at room temperature for 18 hours. Saturated aqueous sodium bicarbonate was added, followed by dichloromethane. Organic layer was separated, dried over sodium sulfate, filtered and concentrated to give 4-tert-butyl-3-fluorophenol (0.544 g, 70%).

(6H): m/z (M+1)=345.1

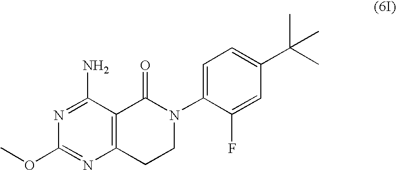

### 4-amino-6-(4-tert-butyl-2-fluorophenyl)-2-methoxy-7,8-dihydropyrido[4,3-d]pyrimidin-5(6H)-one

Prepared analogous to (6A) from 4-tert-butyl-2-fluorophenol which was prepared as follows:

4-Tert-butylphenol (1.1 g, 7.332 mmol) and selectfluor™ (2.84 g, 7.69 mmol) combined in methanol (70 mL) and heated to 80° C. for 24 hours. Reaction cooled to room temperature and solids filtered off. Filtrate concentrated and purified on silica gel eluting with a gradient from 0% to 25% ethyl acetate in heptane to give 4-tert-butyl-2-fluorophenol (450 mg, 36%).

1H NMR (400 MHz, CHLOROFORM-d) δ ppm 1.26 (s, 9H) 4.98 (s, 1H) 6.91 (d, 1H) 7.00-7.03 (m, 1H) 7.07 (dd, 1H)

(6I): 1H NMR (400 MHz, CHLOROFORM-d) δ ppm 1.31 (s, 9H) 3.06 (t, J=6.73 Hz, 2H) 3.85 (t, J=6.83 Hz, 2H) 3.94 (s, 3H) 5.50 (br. s., 1H) 7.14-7.23 (m, 3H) 8.57 (br. s., 1H)

m/z (M+1)=345.5

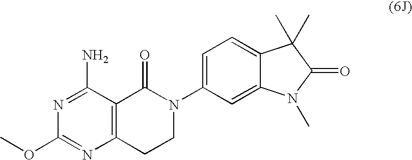

### 4-amino-2-methoxy-6-(1,3,3-trimethyl-2-oxo-2,3-dihydro-1H-indol-6-yl)-7,8-dihydropyrido[4,3-d]pyrimidin-5(6H)-one

Prepared analogous to (6A) from 6-hydroxy-1,3,3-trimethylindolin-2-one which was prepared as follows:

To a solution of 3-nitro-4-bromoanisole (25.0 g, 0.1077 mol) in ethanol (360 mL) was added iron (19.2 g, 0.345 mol) and 37% aqueous hydrochloric acid (44.4 mL). The mixture was allowed to self heat to reflux and then reflux maintained for 2.5 hours. The reaction mixture was cooled and made basic with saturated aqueous sodium bicarbonate. Ethyl acetate was then added and the mixture filtered through dicalite and the organic phase separated. The organic phase was dried over magnesium sulfate, filtered and concentrated. The resulting residue was dissolved in dichloromethane (250 mL) and triethylamine (22.5 mL, 0.162 mol)) added. Reaction cooled to 0° C. and isobutyryl chloride (0.119 mol) was added and the mixture stirred at room temperature for 72 hours. The reaction mixture was washed with 2M hydrochloric acid and saturated aqueous sodium bicarbonate, dried over magnesium sulfate and evaporated to give N-(2-bromo-5-methoxyphenyl)isobutyramide (25.0 g, 85% yield).

1H NMR (CDCl3): δ ppm 8.12 (d, 1H), 7.71 (bs, NH), 7.37 (d, 1H), 6.55 (dd, 1H), 3.80 (s, 3H), 2.70-2.55 (m, 1H), 1.29 (d, 6H)

To a solution of N-(2-bromo-5-methoxyphenyl)isobutyramide (12.5 g, 0.0461 mol) in dimethylformamide (100 mL) was added iodomethane (3.3 mL, 0.053 mol), followed by sodium hydride (60% in oil, 2.5 g, 0.0625 mol) over 15 minutes and stirred at room temperature for 2 hours. The reaction mixture was carefully poured into ice/water and extracted with ethyl acetate. The organic phase was washed with 1:1 brine/water, dried over magnesium sulfate and concentrated. Crude purified on silica gel eluting with 25% ethyl acetate in heptane to give N-(2-bromo-5-methoxyphenyl)-N-methylisobutyramide (10.4 g, 79% yield).

1H NMR (CDCl3): δ ppm 7.55-7.52 (m, 1H), 7.82-7.77 (m, 2H), 3.80 (s, 3H), 3.16 (s, 3H), 2.28 (dt, 1H), 1.08 (d, 3H), 1.01 (d, 3H)

To degassed anhydrous dioxane (50 mL) was added tricyclohexylphosphine (510 mg, 1.8 mmol), palladium (II) acetate (410 mg, 1.8 mmol) and sodium tert butoxide (5.2 g, 54.5 mmol). N-(2-bromo-5-methoxyphenyl)-N-methylisobutyramide (10.4 g, 36.3 mmol) was added and the mixture heated at reflux for 10 hours. The reaction was cooled to room temperature, diluted with ethyl acetate and washed with saturated aqueous ammonium chloride. The organic phase was dried over magnesium sulfate, filtered and concentrated. Crude purified on silica gel eluting with a gradient from, evaporated amd columned on silica eluting with a gradient from 25% to 33% ethyl acetate in heptane to give 6-methoxy-1,3,3-trimethylindolin-2-one (5.58 g, 75%).

m/z (M+1)=206.1

1H NMR (CDCl3): δ ppm 1.33 (s, 6H), 3.19 (s, 3H), 3.82 (s, 3H), 6.43(d, 1H),6.55 (dd, 1H), 7.08 (d, 1H)

To a solution of 6-methoxy-1,3,3-trimethylindolin-2-one (5.57 g, 0.0271 mol) in dichloromethane (75 mL) at −10° C. was added boron tribromide (5.56 mL, 0.0325 mol) over 15 minutes and stirred for 2 hours at −10° C. The reaction mixture was poured into ice/water and made basic with saturated aqueous sodium bicarbonate. The organic phase was separated, dried over magnesium sulfate and concentrated. The residue was purified on silica eluting with 25% ethyl acetate in hexane to give 6-hydroxy-1,3,3-trimethylindolin-2-one (4.74 g, 90%).

1H NMR (d6-DMSO): δ ppm 1.16 (s, 6H), 3.05 (s, 3H), 6.33-6.40 (m, 2H), 7.03 (d, 1H), 9.42 (OH)

(6J): 1H NMR (d6-DMSO): δ ppm 1.25 (s, 6H), 2.92 (t, 2H), 3.09 (s, 3H), 3.82 (s, 3H), 3.87 (t, 2H), 6.97 (dd, 1H), 7.01 (d, 1H), 7.34 (d, 1H), 7.78 (NH), 8.34 (NH)

m/z (M+1)=368.0

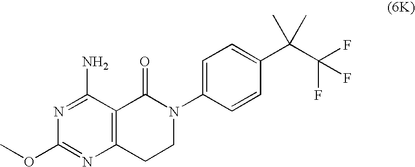

### 4-amino-2-methoxy-6-[4-(2,2,2-trifluoro-1,1-dimethylethyl)phenyl]-7,8-dihydropyrido[4,3-d]pyrimidin-5(6H)-one

Prepared analogous to (6A) from 4-(1,1,1-trifluoro-2-methylpropan-2-yl)phenol which was prepared as follows:

Piperidine (99.8 mL, 1.01 mol, 1.25 eq) and triethylamine (120.8 mL, 0.81 mol, 1.0 eq) in ether (394 mL) were cooled to 0° C. and trifluoroacetic anhydride (120.8 mL, 0.81 mol, 1.0 eq) in ether (263 mL) was added drop wise over 30 minutes. The reaction was warmed to room temperature and stirred for 16 hours. The reaction was diluted with ether (625 mL) and washed with 0.2 N aqueous hydrochloric acid until neutral. The organic portion was washed with brine, dried over sodium sulfate and concentrated. The resulting yellow oil was purified on silica gel eluting with 10% ethyl acetate in hexane to give 2,2,2-trifluoro-1-(piperidin-1-yl)ethanone (140.35 g, 77%).

1H NMR (CDCl3, 400 MHz): δ ppm 3.61 (2H, m), 3.52 (2H, m), 1.67 (6H, m).

Magnesium turnings (7.73 g, 318 mmol, 1.25 eq) and tetrahydrofuran (63 mL) were placed in a 3 neck flask. 4-Bromoanisole (59.40 g, 318 mmol, 1.25 eq) in tetrahydrofuran (63 mL) was added drop wise and the flask heated until a vigorous reaction occurred. Once the magnesium had dissolved the reaction was cooled to 0° C. and 2,2,2-trifluoro-1-(piperidin-1-yl)ethanone (46.00 g, 258 mmol) in tetrahydrofuran (250 mL) was added drop wise. The reaction was stirred at room temperature for 2 hours and was subsequently quenched with saturated aqueous ammonium chloride and the resulting precipitate filtered off. The filtrate was dried over sodium sulfate concentrated to give an orange oil which was purified by distillation (120 C, 32 mbar) to give 2,2,2-trifluoro-1-(4-methoxyphenyl)ethanone (80 g, 52%).

2,2,2-Trifluoro-1-(4-methoxyphenyl)ethanone (80.00 g, 392 mmol) in diethyl ether (800 mL) was cooled to 0° C. Methyl magnesium bromide (3.0M in diethyl ether, 130.4 mL, 392 mmol, 1.0 eq) was added drop wise and the reaction allowed to warm to room temperature overnight. The reaction was quenched with 1N hydrochloric acid (800 mL), the layers separated and the organic portion washed with water (800 mL) dried over sodium sulfate and concentrated to give 1,1,1-trifluoro-2-(4-methoxyphenyl)propan-2-ol (85 g, 98%) as a yellow oil.

1H NMR (CDCl3, 400 MHz): δ ppm 7.50 (2H, d), 6.91 (2H, d), 3.81 (3H, s), 2.33 (1H, bs), 1.75 (3H, s)0.00 MHz): 8.05 (2H, d), 7.00 (2H, d), 3.90 (3H, s).

1,1,1-Trifluoro-2-(4-methoxyphenyl)propan-2-ol (85.00 g, 391 mmol) in dichloromethane (860 mL) was cooled to 0° C. and titanium tetrachloride (40.52 mL, 1.0 eq) was added slowly to the reaction. The reaction was stirred at 0° C. for 1.5 hours and was then added slowly to ice water and the layers were separated and the aqueous portion extracted with dichloromethane (3×500 mL). The combined organics were washed with saturated sodium hydrogen carbonate and brine, dried over sodium sulfate and concentrated. The crude oil was purified on silica gel eluting with hexane to give 1-(2-chloro-1,1,1-trifluoropropan-2-yl)-4-methoxybenzene (60.9 g, 65%).

1H NMR (CDCl3, 400 MHz): δ ppm 7.58 (2H, d), 6.89 (2H, d), 3.78 (3H, s), 2.11 (3H, s).

Trimethyl aluminium (2.0 M in heptane, 504 mL, 1.04 mol, 4 eq) was added to 1-(2-chloro-1,1,1-trifluoropropan-2-yl)-4-methoxybenzene (60.00 g, 251 mmol) in hexane (840 mL). The reaction was heated at reflux for 2 hours. The reaction was cooled and quenched slowly with 2N hydrochloric acid. The layers were separated and the aqueous portion extracted with hexane. The organic portion was dried over sodium sulfate and concentrated to give 1-methoxy-4-(1,1,1-trifluoro-2-methylpropan-2-yl)benzene (32.09 g, 58%).

1H NMR (CDCl3, 400 MHz): 7.42 (2H, d), 6.90 (2H, d), 3.79 (3H, s), 1.55 (6H, s).

1-Methoxy-4-(1,1,1-trifluoro-2-methylpropan-2-yl)benzene (32.00 g, 147 mmol) in dichloromethane (500 mL) was cooled to 0° C. Boron tribromide (14.14 mL, 147 mmol, 1.0 eq) was added drop wise. The reaction was allowed to warm to room temperature and was stirred for 4 hours. The reaction was then cooled to 0° C. and quenched by the slow addition of water. The layers were separated and the aqueous portion extracted with dichloromethane. The combined organic extracts were washed with brine and dried over sodium sulfate and concentrated. Crude was purified on silica gel eluting with 5% ethyl acetate in hexane to give 4-(1,1,1-trifluoro-2-methylpropan-2-yl)phenol (29.01 g, 97%).

1H NMR (CDCl3, 400 MHz): δ ppm 7.34 (2H, d), 6.82 (2H, d), 1.53 (6H, s).

(6K): 1H NMR (400 MHz, CDCl3): δ ppm 8.60 (bs, 1H), 7.55 (d, J=8.8, 2H), 7.32 (d, J=8.8, 2H), 5.54 (bs, 1H), 3.95 (m, 5H), 3.05 (t, J=6.6, 2H), 1.58 (6H, s)

m/z (M+1)=381.3

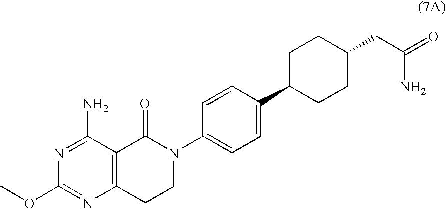

### 2-{trans-4-[4-(4-amino-2-methoxy-5-oxo-7,8-dihydropyrido[4,3-d]pyrimidin-6(5H)-yl)phenyl]cyclohexyl}acetamide

Prepared analogous to the (6A) from methyl 2-((1r,4r)-4-(4-hydroxyphenyl)cyclohexyl)acetate via the following synthesis:

Methyl 2-((1r,4r-4-(4-(4-amino-2-methoxy-5-oxo-7,8-dihydropyrido[4,3-d]pyrimidin-6(5H)-yl)phenyl)cyclohexyl)acetate (5.38 g, 12.67 mmol) and lithium hydroxide (2.13 g, 50.7 mmol) in methanol (100 mL), tetrahydrofuran (150 mL) and water (50 mL) was stirred at room temperature for 18 hours. Reaction was then heated to 50° C. and stirred for 2 hours. Reaction mixture cooled to room temperature and concentrated to ˜ 1/10 of the initial volume. 1M aqueous hydrochloric acid (45 mL), 0.5M aqueous citric acid (20 mL) and water (100 mL) added and stirred for 1 hour. Solids filtered off, washing with water. Solids were then stirred in methanol (150 mL) at 85° C. for 1 hour and then cooled to room temperature and stirred for 2 hours. Solids filtered off and dried under high vacuum to give 2-((1r,4r)-4-(4-(4-amino-2-methoxy-5-oxo-7,8-dihydropyrido[4,3-d]pyrimidin-6(5H)-yl)phenyl)cyclohexyl)acetic acid (7B) (4.7 g, 90%).

1H NMR (500 MHz, DMSO-d6) δ ppm 1.07-1.19 (m, 2H) 1.41-1.53 (m, 2H) 1.70-1.77 (m, 1H) 1.77-1.87 (m, 4H) 2.15 (d, 2H) 2.43-2.49 (m, 1H) 2.94 (t, 2H) 3.85 (s, 3H) 3.87 (t, 2H) 7.22-7.28 (m, 4H) 7.77 (d, 1H) 8.38 (d, 1H) 12.01 (s, 1H)

(7B) (4.7 g, 11.45 mmol), diisopropylethylamine (2.8 ml, 16.0 mmol) and benzotriazole-1-yl-oxy-tris-(dimethylamino)-phosphonium hexafluorophosphate (6080 mg, 13.70 mmol) were combined in dimethylformamide (75 mL) and stirred at room temperature for 1 hour. 4-Methoxybenzylamine (1.8 ml, 13.70 mmol) was added and the reaction was stirred at room temperature for 2 hours. Water (400 mL) was added and stirred for 10 minutes. Methyl tert-butyl ether (100 mL) added and stirred another 10 minutes. Solids filtered off, washed with methyl tert-butyl ether of water and dried in a vacuum oven at 45° C. to give N-(4-methoxybenzyl)-2-((1r,4r)-4-(4-(4-amino-2-methoxy-5-oxo-7,8-dihydropyrido[4,3-d]pyrimidin-6(5H)-yl)phenyl)cyclohexyl)acetamide (6.1 g, 100%).

1H NMR (500 MHz, DMSO-d6) δ ppm 1.05-1.14 (m, 2H) 1.40-1.50 (m, 2H) 1.74-1.82 (m, 5H) 2.06 (d, 2H) 2.43-2.50 (m, 1H) 2.94 (t, 2H) 3.72 (s, 3H) 3.84 (s, 3H) 3.86 (t, 2H) 4.20 (d, 2H) 6.88 (d, 2H) 7.17 (d, 2H) 7.23-7.27 (m, 4H) 7.79 (d, 1H) 8.25 (t, 1H) 8.38 (d, 1H)

N-(4-methoxybenzyl)-2-((1r,4r)-4-(4-(4-amino-2-methoxy-5-oxo-7,8-dihydropyrido[4,3-d]pyrimidin-6(5H)-yl)phenyl)cyclohexyl)acetamide (6.1 g, 11.52 mmol) in trifluoroacetic acid (35 mL, 470 mmol) and heated to 80° C. for 18 hours. Reaction concentrated to dryness and toluene (50 mL) added and concentrated again (2×). A small amount of the residue was stirred with a mixture of methyl tert-butyl ether (10 mL), saturated aqueous sodium bicarbonate (10 mL) and water (10 mL) at room temperature for 2 hours. The white precipitate that formed was collected and dried in a vacuum oven at 45° C. to give the title compound (7A).

1H NMR (500 MHz, DMSO-d6) δ ppm 1.04-1.13 (m, 2H) 1.40-1.48 (m, 2H) 1.69-1.76 (m, 1H) 1.77-1.83 (m, 4H) 1.97 (d, 2H) 2.44-2.49 (m, 1H) 2.94 (t, 2H) 3.84 (s, 3H) 3.87 (t, 2H) 6.72 (s, 1H) 7.23-7.27 (m, 5H) 7.78 (d, 1H) 8.37 (d, 1H)

m/z (M+1)=410.5

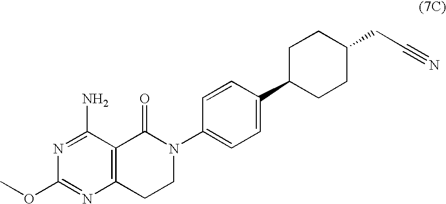

### {trans-4-[4-(4-amino-2-methoxy-5-oxo-7,8-dihydropyrido[4,3-d]pyrimidin-6(5H)-yl)phenyl]cyclohexyl}acetonitrile

(7A) (4.6 g, 11.23 mmol) and phosphorus oxychloride (40 mL) were combined and heated to 70° C. for 1.5 hours. The mixture was concentrated and to the residue, ethyl acetate (20 mL) was added, and basified with 1 M aqueous sodium hydroxide. The mixture was stirred for 1 hour and solids filtered off to give crude product. Crude purified on silica gel eluting with a gradient from 0% to 15% methanol in dichloromethane. Isolated product in dichloromethane was further purified with the addition of activated carbon. Solution stirred at room temperature for 2 hours. The solution was filtered through a small pad of celite and filtrate concentrated. Residue was crystallized from dichloromethane and methyl tert butyl ether to give the title compound (7C).

1H NMR (500 MHz, DMSO-d6) δ ppm 1.17-1.26 (m, 2H) 1.45-1.53 (m, 2H) 1.65-1.74 (m, 1H) 1.81-1.90 (m, 4H) 2.45-2.48 (m, 1H) 2.50 (d, 2H) 2.94 (t, 2H) 3.85 (s, 3H) 3.88 (t, 2H) 7.23-7.28 (m, 4H) 7.78 (d, 1H) 8.37 (d, 1H)

m/z (M+1)=392.3

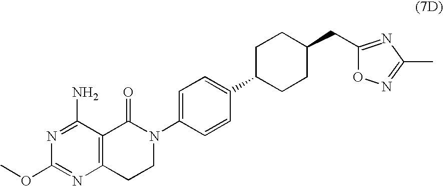

### 4-amino-2-methoxy-6-(4-((1r,4r)-4-((3-methyl-1,2,4-oxadiazol-5-yl)methyl)cyclohexyl)phenyl)-7,8-dihydropyrido[4,3-d]pyrimidin-5(6H)-one

(7B) (100 mg, 0.244 mmol) in thionyl chloride (1 mL, 10 mmol) and dimethylformamide (0.03 mL, 0.4 mmol) was stirred at room temperature for 3 hours. Reaction concentrated and dried under vacuum. Residue was dissolved in tetrahydrofuran (4 mL) and N-methylpyrrolidinone (2 mL) and N-hydroxy-acetamidine (181 mg, 2.44 mmol) was added. Stirred at room temperature for 18 hours. Reaction is concentrated ¼^{th} of the initial volume. Saturated aqueous sodium bicarbonate (10 mL) added and stirred at room temperature for 15 minutes. The solid was filtered off, washed with water, dried under vacuum to give 2-((1r,4r)-4-(4-(4-amino-2-methoxy-5-oxo-7,8-dihydropyrido[4,3-d]pyrimidin-6(5H)-yl)phenyl)cyclohexyl)-N—((Z)-1-(hydroxyimino)ethyl)acetamide (110 mg, 96%). Used in the next step without further purification.

2-((1r,4r)-4-(4-(4-amino-2-methoxy-5-oxo-7,8-dihydropyrido[4,3-d]pyrimidin-6(5H)-yl)phenyl)cyclohexyl)-N—((Z)-1-(hydroxyimino)ethyl)acetamide (110 mg, 0.236 mmol) in dimethylformamide (2 mL) was heated to 140° C. for 2 hours. Reaction cooled to room temperature and water (5 mL) added. Solid filtered off and purified on silica gel, eluting with a gradient from 80% to 100% of ethyl acetate in heptane to give the target compound (7D) (16 mg, 15%) as a white solid.

1H NMR (500 MHz, CHLOROFORM-d) δ ppm 1.21-1.31 (m, 2H) 1.46-1.57 (m, 2H) 1.88-2.00 (m, 5H) 2.41 (s, 3H) 2.48-2.57 (m, 1H) 2.82 (d, 2H) 3.06 (t, 2H) 3.93 (t, 2H) 3.97 (s, 3H) 5.56 (s, 1H) 7.23-7.27 (m, 4H) 8.64 (s, 1H)

m/z (M+1)=449.5

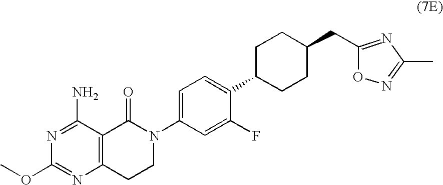

### 4-amino-6-(3-fluoro-4-{trans-4-[(3-methyl-1,2,4-oxadiazol-5-yl)methyl]cyclohexyl}phenyl)-2-methoxy-7,8-dihydropyrido[4,3-d]pyrimidin-5(6H)-one

Prepared analogous to (7D) from methyl 2-(4-(2-fluoro-4-hydroxyphenyl)cyclohexyl)acetate.

(7E): 1H NMR (400 MHz, CHLOROFORM-d) δ ppm 1.13-1.33 (m, 2H) 1.44-1.60 (m, 2H) 1.81-2.04 (m, 5H) 2.38 (s, 3H) 2.75-2.89 (m, 3H) 3.03 (t, J=6.85 Hz, 2H) 3.90 (t, J=6.85 Hz, 2H) 3.94 (s, 3H) 5.53 (br. s., 1H) 6.92-7.09 (m, 2H) 7.17-7.28 (m,1H) 8.56 (br.s.,1H)

m/z (M+1)=467.5

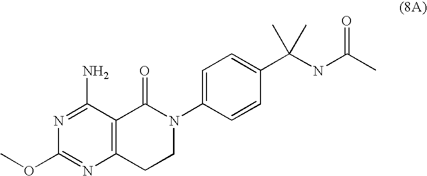

### N-{1-[4-(4-amino-2-methoxy-5-oxo-7,8-dihydropyrido[4,3-d]pyrimidin-6(5H)-yl)phenyl]-1-methylethyl}acetamide

Prepared analogous to (1A) from benzyl 2-(4-bromophenyl)-2-methylpropanoate to give Benzyl 2-(4-(4-amino-2-methoxy-5-oxo-7,8-dihydropyrido[4,3-d]pyrimidin-6(5H)-yl)phenyl)-2-methylpropanoate which was taken through the following synthesis to get to (8A).

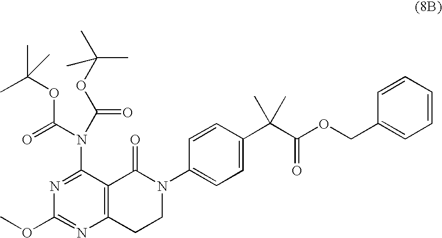

### Bis-BOC protected benzyl 2-(4-amino-2-methoxy-5-oxo-7,8-dihydropyrido[4,3-d]pyrimidin-6(5H)-yl)-2-methylpropanoate

Benzyl 2-(4-(4-amino-2-methoxy-5-oxo-7,8-dihydropyrido[4,3-d]pyrimidin-6(5H)-yl)phenyl)-2-methylpropanoate (100 mg, 0/224 mmol), di-tert-butyl dicarbonate (130 mg, 0.61 mmol) and 4-dimethylaminopyridine (7 mg, 0.06 mmol) were combined in tetrahydrofuran (5 mL) and stirred at room temperature for 24 hours. Reaction concentrated and purified on silica, eluting with a gradient from 30 to 70% ethyl acetate in heptane to give (8B). Used as is in the following reaction without further purification.

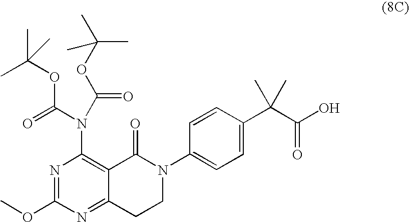

### Bis-BOC protected 2-(4-amino-2-methoxy-5-oxo-7,8-dihydropyrido[4,3-d]pyrimidin-6(5H)-yl)-2-methylpropanoic acid

(8B) (100 mg, 0.155 mmol) in tetrahydrofuran (30 mL) was added 10% palladium on activated carbon (50 mg) and allowed to stir in a parr shaker at room temperature under hydrogen (45 PSI) for 2 hours. Reaction mixture was carefully filtered through a pad of celite under a steady stream of nitrogen washing with copious amounts of ethyl acetate Filtrate concentrated to (8C) (82 mg, 95%) as a white solid.

1H NMR (400 MHz, CHLOROFORM-d) δ ppm 1.44 (s, 18H) 1.59 (s, 6H) 3.20 (t, 2H) 3.96 (t, 2H) 4.04 (s, 3H) 7.28 (d, 2H) 7.40 (d, 2H)

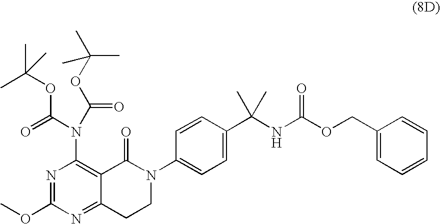

### Bis-BOC protected benzyl 2-(4-amino-2-methoxy-5-oxo-7,8-dihydropyrido[4,3-d]pyrimidin-6(5H)-yl)propan-2-ylcarbamate

(8C) (80 mg, 0.14 mmol), diphenyl phosphoryl azide (0.048 mL, 0.216 mmol), triethylamine (0.03 mL, 0.216 mmol), and 100 mg of 4A molecular sieves were combined in toluene (5 mL) and heated to reflux (110° C.) for 2 hours. Reaction cooled to room temperature and benzyl alcohol (0.3 mL, 2.88 mmol) added and heated back to reflux (110° C.) for 18 hours. Solids were filtered and the filtrate concentrated to give a crude residue that was purified on silica gel eluting with a gradient from 30 to 90% ethyl acetate in heptane to give (8D) (50 mg, 52%).

1H NMR (400 MHz, CHLOROFORM-d) δ ppm 1.43 (s, 18H) 1.65 (s, 6H) 3.20 (t, 2H) 3.96 (t, 2H) 4.04 (s, 3H) 5.01 (s, 2H) 5.16 (s, 1H) 7.24-7.41 (m, 9H)

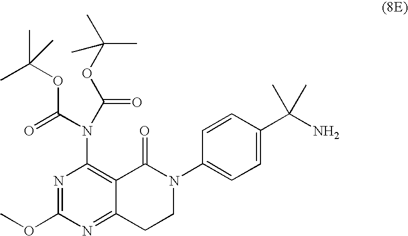

### Bis-BOC protected 4-amino-6-(2-aminopropan-2-yl)-2-methoxy-7,8-dihydropyrido[4,3-d]pyrimidin-5(6H)-one

(8D) (48 mg, 0.073 mmol) in tetrahydrofuran (30 mL) was added 10% palladium on activated carbon (30 mg)) and allowed to stir in a parr shaker at room temperature under hydrogen (45 PSI) for 72 hours. Reaction mixture was carefully filtered through a pad of celite under a steady stream of nitrogen washing with copious amounts of ethyl acetate Filtrate concentrated to give (8E) (32 mg, 99%) as a white solid.

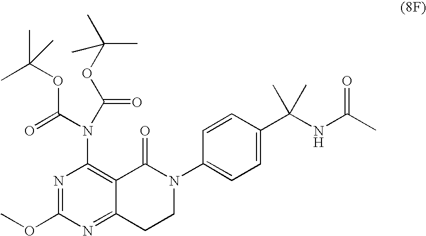

### Bis-BOC protected N-(2-(4-amino-2-methoxy-5-oxo-7,8-dihydropyrido[4,3-d]pyrimidin-6(5H)-yl)propan-2-yl)acetamide

(8E) (19 mg, 0.036 mmol) and triethylamine (0.08 mL, 0.6 mmol) were dissolved in dichloromethane (2 mL). Acetic anhydride (30 mg, 0.29 mmol) was added and reaction stirred for 3 hours. Reaction was concentrated and purified on silica gel eluting with a gradient from 0% to 8% methanol in ethyl acetate to give (8F) (9 mg, 40%).

(8F) (9 mg, 0.02 mmol) in methanol (1 mL) was added 4M hydrochloric acid in 1,4-dioxane (1 mL) and stirred at room temperature for 72 hours. Reaction concentrated and purified on a reverse phase column using the following conditions:

Preparative LC/MS method conditions:

MS mode: MS:ESI+scan range 160-850 daltons

Column: Waters Atlantis dC18 19×100 mm, 5 um

Modifier: Formic acid 0.05%

Method: 95% H20/5% MeCN (initial conditions) linear gradient to 70% H20/30% MeCN at 7.0 min, ramp to 5% H20/95% MeCN at 7.5 min, HOLD 5% H20/95% MeCN to 8.5 min. Flow rate, 25 mL/min.

QC Analysis method conditions:

MS mode: MS:ESI+ scan range 160-850 daltons

Column: Waters Xbridge C18 4.6×50 mm, 5 um

Modifier: Ammonium hydroxide 0.03%

Method: 95% H20/5% MeCN (initial conditions) linear gradient to 5% H20/95% MeCN at 4.0 min, HOLD 5% H20/95% MeCN to 5 min. Flow rate, 2 mL/min. to give the taget compound, (8A).

m/z (M+1)=370.7

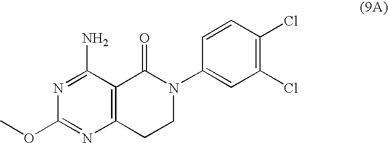

### 4-amino-6-(3,4-dichlorophenyl)-2-methoxy-7,8-dihydropyrido[4,3-d]pyrimidin-5(6H)-one

3,4-Dichloroaniline (550 mg, 3.4 mmol), ethyl propenoate (0.406 mL, 3.74 mmol) and triethylamine (0.24 mL, 3.5 mmol) in ethanol (0.25 mL) was heated to 100° C. for 72 hours. Reaction mixture was concentrated and toluene (20 mL) was added and concentrated (repeated twice) to give ethyl 3-(3,4-dichlorophenylamino)propanoate which was used in the following step without further purification.

Ethyl 3-(3,4-dichlorophenylamino)propanoate (890 mg, 3.4 mmol), cyanoacetic acid (346 mg, 4.07 mmol) and 4-dimethylaminopyridine (41.5 mg, 0.340 mmol) were combined in dimethylformamide (6 mL) and cooled to 0° C. Diisopropylcarbodiimide (0.631 mL, 4.07 mmol) added drop wise and once addition was complete, reaction was warmed up to room temperature and stirred for 2 hours. Water (50 mL) was added and reaction was extracted with ethyl acetate. Organic layer was washed with water and brine, dried over magnesium sulfate, filtered and concentrated. Crude purified on silica gel, eluting with a gradient from 10% to 70% ethyl acetate to give ethyl 3-(2-cyano-N-(3,4-dichlorophenyl)acetamido)propanoate (610 mg, 54%).

1H NMR (500 MHz, CHLOROFORM-d) δ ppm 1.24 (t, 3H) 2.59 (t, 2H) 3.24 (s, 2H) 4.01 (t, 2H) 4.10 (q, 2H) 7.16 (dd, 1H) 7.41 (d, 1H) 7.59 (d, 1H)

Ethyl 3-(2-cyano-N-(3,4-dichlorophenyl)acetamido)propanoate (610 mg, 1.85 mmol) was dissolved in methanol (20 mL) and 1,8-diazabicycloundec-7-ene (439 mg, 2.88 mmol) was added and the mixture was heated to 80° C. for 1 hour. Reaction was cooled, concentrated and water (20 mL), ethyl acetate (20 mL) and aqueous 1M hydrochloric acid (4 mL) added successively at room temperature and stirred for 30 minutes. The organic phase was separated, washed with brine, dried over magnesium sulfate, filtered and concentrated to give 1-(3,4-dichlorophenyl)-4-hydroxy-2-oxo-1,2,5,6-tetrahydropyridine-3-carbonitrile (345 mg, 65%) as a white solid.

1H NMR (500 MHz, DMSO-d6) δ ppm 2.82 (t, 2H) 3.81 (t, 2H) 7.30 (dd, 1H) 7.60 (d, 1H) 7.63 (d, 1H)

1-(3,4-dichlorophenyl)-4-hydroxy-2-oxo-1,2,5,6-tetrahydropyridine-3-carbonitrile (345 mg, 1.22 mmol) in dimethylformamide (0.007 mL, 0.092 mmol) and dichloromethane (14 mL) was cooled to 0° C. Oxalyl chloride (0.271 mL, 3.05 mmol) was added drop wise. Once addition was complete, reaction was slowly warmed to room temperature and stirred for 24 hours. Reaction was concentrated and toluene (25 mL) added and concentrated to dryness. Methanol (15 ml) was added and stirred at 65° C. for 18 hours. Reaction concentrated to give 4-amino-6-(3,4-dichlorophenyl)-2-methoxy-7,8-dihydropyrido[4,3-d]pyrimidin-5(6H)-one (365 mg, 100%) which was used in the next step without further purification.

1H NMR (400 MHz, DMSO-d6) δ ppm 3.04 (t, 2H) 3.85 (t, 2H) 4.02 (s, 3H) 7.30 (dd, 1H) 7.60 (d, 1H) 7.61 (d, 1H)

4-amino-6-(3,4-dichlorophenyl)-2-methoxy-7,8-dihydropyrido[4,3-d]pyrimidin-5(6H)-one (365 mg, 1.23 mmol) dissolved in methanol (10 mL) and O-methylisourea hydrochloride (380 mg, 3.44 mmol) and diisopropylethylamine (0.749 mL, 4.3 mmol) were added and heated to 80° C. 1 hour. Reaction was concentrated and purified on silica, eluting with a gradient from 50 to 100% ethyl acetate in heptane to give the target compound (9A) (170 mg, 40%) as a colorless solid.

1H NMR (400 MHz, DMSO-d6) δ ppm 2.92 (t, 2H) 3.82 (s, 3H) 3.89 (t, 2H) 7.35 (dd, 1H) 7.63 (d, 1H) 7.66 (d, 1H) 7.82 (d, 1H) 8.25 (d, 1H)

m/z (M+1)=339.0

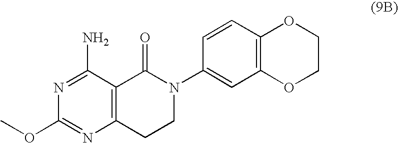

### 4-amino-6-(2,3-dihydro-1,4-benzodioxin-6-yl)-2-methoxy-7,8-dihydropyrido[4,3-d]pyrimidin-5(6H)-one

Prepared analogous to (9A) from the commercially available 2,3-dihydro-1,4-benzodioxin-7-amine.

(9B): 1H NMR (400 MHz, CHLOROFORM-d) δ ppm 3.00 (t, 2H) 3.83 (t, 2H) 3.92 (s, 3H) 4.23 (s, 4H) 5.61 (s, 1H) 6.74 (dd, 1H) 6.80 (d, 1H) 6.86 (d, 1H) 8.61 (s, 1H)

m/z (M+1)=329.1

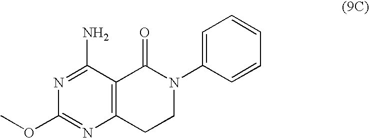

### 4-amino-2-methoxy-6-phenyl-7,8-dihydropyrido[4,3-d]pyrimidin-5(6H)-one

Prepared analogous to (9A) from the commercially available aniline.

(9C): 1H NMR (500 MHz, DMSO-d6) δ ppm 2.96 (t, 2H) 3.85 (s, 3H) 3.90 (t, 2H) 7.22-7.27 (m, 1H) 7.33-7.43 (m, 4H) 7.79 (d, 1H) 8.37 (d, 1H)

m/z (M+1)=271.1

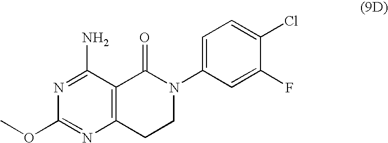

### 4-amino-6-(4-chloro-3-fluorophenyl)-2-methoxy-7,8-dihydropyrido[4,3-d]pyrimidin-5(6H)-one

Prepared analogous to (9A) from the commercially available 3-fluoro-4-chloroaniline.

(9D): 1H NMR (400 MHz, CHLOROFORM-d) δ ppm 3.05 (t, J=6.73 Hz, 2H) 3.91 (t, J=6.73 Hz, 2H) 3.95 (s, 3H) 5.57 (br. s., 1H) 7.03-7.08 (m, 1H) 7.17 (dd, J=10.15, 2.54 Hz, 1H) 7.41 (t, J=8.39 Hz, 1H) 8.51 (br. s., 1H)

m/z (M+1)=323.0

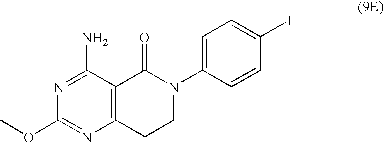

### 4-amino-6-(4-iodophenyl)-2-methoxy-7,8-dihydropyrido[4,3-d]pyrimidin-5(6H)-one

Prepared analogous to (9A) from the commercially available 4-iodobenzenamine.

(9E): m/z (M+1)=397.0

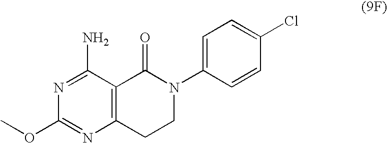

### 4-amino-6-(4-chlorophenyl)-2-methoxy-7,8-dihydropyrido[4,3-d]pyrimidin-5(6H)-one

Prepared analogous to (9A) from the commercially available 4-chloroaniline.

(9F): 1H NMR (CDCl3) δ ppm 8.54 (bs, 1H), 8.37 (s, 1H), 7.81 (d, J=8.8, 1H), 7.67 (d, J=8.8, 1H), 5.62 (bs, 1HHH), 4.22 (t, J=6.8, 2H), 3.95 (s, 3H), 3.02 (t, J=6.8, 2H)

m/z (M+1)=306.4

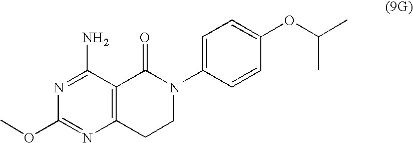

### 4-amino-6-(4-isopropoxyphenyl)-2-methoxy-7,8-dihydropyrido[4,3-d]pyrimidin-5(6H)-one

Prepared analogous to (9A) from the commercially available 4-isopropoxyaniline.

(9G): 1H NMR (500 MHz, CHLOROFORM-d) δ ppm 1.35 (d, 6H) 3.06 (t, 2H) 3.91 (t, 2H) 3.95-3.98 (m, 3H) 4.51-4.59 (m, 1H) 5.61 (s, 1H) 6.93 (d, 2H) 7.21 (d, 2H) 8.67 (s, 1H)

m/z (M+1)=329.2

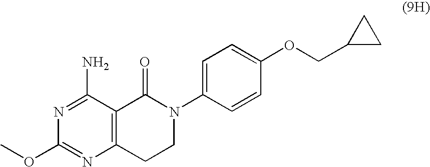

### 4-amino-6-[4-(cyclopropylmethoxy)phenyl]-2-methoxy-7,8-dihydropyrido[4,3-d]pyrimidin-5(6H)-one

Prepared analogous to (9A) from 4-(cyclopropylmethoxy)benzenamine which was prepared as follows:

4-Nitrophenol (900 mg, 6.47 mmol), cyclopropyl carbinol (700 mg, 9.7 mmol), triphenylphosphine (2.55 g, 9.7 mmol) combined in tetrahydrofuran (25 mL) and cooled to 0° C. Diisopropyl azodicarboxylate (1.96 g, 9.7 mmol) added drop wise. Once addition was complete, the reaction was warmed to room temperature and stirred for 10 hours. Reaction was concentrated and purified on silica gel, eluting with a gradient from 10% to 50% ethyl acetate in heptane to give 1-(cyclopropylmethoxy)-4-nitrobenzene (1.24 g, 99%).

1H NMR (400 MHz, CHLOROFORM-d) δ ppm 0.34-0.40 (m, 2H) 0.65-0.71 (m, 2H) 1.23-1.33 (m, 1H) 3.88 (d, 2H) 6.93 (d, 2H) 8.18 (d, 2H)

1-(cyclopropylmethoxy)-4-nitrobenzene (1.24 g, 6.418 mmol) in ethyl acetate (40 mL) was added 10% palladium on acivated carbon (50 mg) and allowed to stir in a parr shaker at room temperature under hydrogen (45 PSI) for 2 hours. Reaction mixture was carefully filtered through a pad of celite under a steady stream of nitrogen washing with ethyl acetate. Filtrate concentrated to give 4-(cyclopropylmethoxy)benzenamine (1.04 g, 99%) as a colorless oil, which was used for the next step without further purification.

(9H): 1H NMR (400 MHz, CHLOROFORM-d) δ ppm 0.31-0.36 (m, 2H) 0.60-0.67 (m, 2H) 1.22-1.29 (m, 1H) 3.06 (t, 2H) 3.79 (d, 2H) 3.88 (t, 2H) 3.95 (s, 3H) 5.57 (s, 1H) 6.92 (d, 2H) 7.19 (d, 2H) 8.69 (s, 1H)

m/z (M+1)=341.2

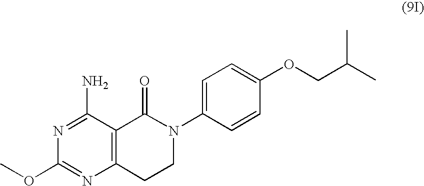

### 4-amino-6-(4-isobutoxyphenyl)-2-methoxy-7,8-dihydropyrido[4,3-d]pyrimidin-5(6H)-one

Prepared analogous to (9A) from 4-isobutoxybenzenamine which was prepared as follows:

4-Nitrophenol (1.15 g, 8.267 mmol), 1-iodo-2-methylpropane (1.44 mL, 12.4 mmol) and cesium carbonate (4.04 g, 12.4 mmol) were combined in dimethylformamide (12 mL) and stirred at room temperature for 18 hours and then heated to 60° C. for 24 hours. Water (50 ml) was added and the reaction was extracted with 1:1 ethyl acetate:heptane. Organics were washed with concentrated aqueous sodium carbonate (3×), brine, dried over magnesium sulfate, filtered and concentrated to give 1-isobutoxy-4-nitrobenzene (840 mg, 52%).

1H NMR (500 MHz, CHLOROFORM-d) δ ppm 1.06 (d, 6H) 2.09-2.20 (m, 1H) 3.83 (d, 2H) 6.96 (d, 2H) 8.21 (d, 2H)

1-isobutoxy-4-nitrobenzene (830 mg, 4.25 mmol) in ethyl acetate (40 mL) was added 10% palladium on acivated carbon (50 mg) and allowed to stir in a parr shaker at room temperature under hydrogen (45 PSI) for 2 hours. Reaction mixture was carefully filtered through a pad of celite under a steady stream of nitrogen washing with ethyl acetate. Filtrate concentrated to give 4-isobutoxybenzenamine (700 mg, 99%) as a colorless oil, which was used for the next step without further purification.

1H NMR (400 MHz, CHLOROFORM-d) δ ppm 0.99 (d, 6H) 1.96-2.08 (m, 1H) 3.36 (br. s., 2H) 3.63 (d, 2H) 6.63 (d, 2H) 6.73 (d, 2H)

(9I): 1H NMR (500 MHz, CHLOROFORM-d) δ ppm 1.03 (d, 6H) 2.06-2.14 (m, 1H) 3.06 (t, 2H) 3.73 (d, 2H) 3.90 (t, 2H) 3.96 (s, 3H) 5.63 (s, 1H) 6.95 (d, 2H) 7.21 (d, 2H) 8.67 (s, 1H)

m/z (M+1)=343.2

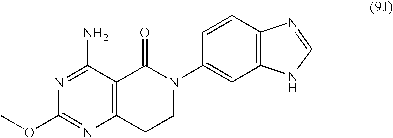

### 4-amino-6-(1H-benzimidazol-6-yl)-2-methoxy-7,8-dihydropyrido[4,3-d]pyrimidin-5(6H)-one

Prepared analogous to (9A) from 3H-benzo[d]imidazol-5-amine which was prepared as follows:

5-Nitrobenzimidazole (1.9444 g, 11.919 mmol) dissolved in acetonitrile (60 mL) and dimethylformamide (7 mL) and cooled to −10° C. 60% sodium hydride in mineral oil (1 g, 25 mmol) added portion wise over 15 minutes then benzyl chloromethyl ether (3.92 g, 25 mmol) was slowly added to cold reaction mixture. Once addition was complete, reaction was slowly warmed up to room temperature and stirred for 1.5 hours. Reaction was concentrated and diluted with water and ethyl acetate. Organic separated, dried over magnesium sulfate, filtered and concentrated to give a mixture of 1-(benzyloxymethyl)-6-nitro-1H-benzo[d]imidazole and 1-(benzyloxymethyl)-5-nitro-1H-benzo[d]imidazole. Crude mixture was used in the following step with out further purification.

Palladium on carbon (95.7 mg) was weighed into a parr shaker bottle and water (5 mL) and ethyl acetate (5 mL) added. Mixture of the crude nitro material (3.3 g, 11.65 mmol) dissolved in ethyl acetate (25 mL) was added slowly to reaction vessel under a steady stream of nitrogen. Reaction set on parr shaker at room temperature under hydrogen (45 psi) for 72 hours. Reaction mixture was carefully filtered through a pad of celite under a steady stream of nitrogen washing with copious amounts of ethyl acetate. Filtrate concentrated and resulting oil purified on silica gel, eluting with a gradient from 0% to 5% methanol in a 1:1 solution of ethyl acetate:dichloromethane to give 3-(benzyloxymethyl)-3H-benzo[d]imidazol-5-amine (813.6 mg, 27%) and 1-(benzyloxymethyl)-1H-benzo[d]imidazol-5-amine (635 mg, 21%). 3-(benzyloxymethyl)-3H-benzo[d]imidazol-5-amine was used to prepare (9J) as the BOM protected bezimidazole. The following de-protection was used to afford 4-amino-6-(1H-benzimidazol-6-yl)-2-methoxy-7,8-dihydropyrido[4,3-d]pyrimidin-5(6H)-one:

4-amino-6-(3-(benzyloxymethyl)-3H-benzo[d]imidazol-5-yl)-2-methoxy-7,8-dihydropyrido[4,3-d]pyrimidin-5(6H)-one (40 mg, 0.093 mmol) dissolved in methanol (2 mL) and tetrahydrofuran (1 mL) was carefully added palladium hydroxide (1.3 mg, 0.009 mmol) in methanol. Reaction set on parr shaker at room temperature under hydrogen (45 PSI) for 18 hours. 1% Hydrogen chloride in methanol (3 mL) was added and set back on parr shaker at room temperature under hydrogen (45 PSI) for 18 hours. Reaction mixture was carefully filtered through a pad of celite under a steady stream of nitrogen washing with 20% methanol in dichloromethane to give the target product (9J) (25 mg, 77%) as a light yellow solid.

1H NMR (400 MHz, METHANOL-d4) δ ppm 3.26 (s, 2H) 4.13 (s, 2H) 4.16 (s, 3H) 7.67 (dd, J=8.88, 1.85 Hz, 1H) 7.91 (s, 2H) 9.44 (s, 1H)

m/z (M+1)=311.0

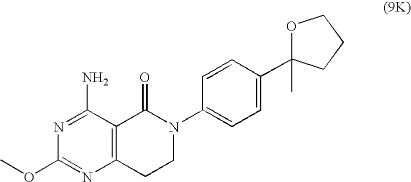

### 4-amino-2-methoxy-6-[4-(2-methyltetrahydrofuran-2-yl)phenyl]-7,8-dihydropyrido[4,3-d]pyrimidin-5(6H)-one

Prepared analogous to (9A) from 4-(2-methyl-tetrahydrofuran-2-yl)benzenamine which was prepared as follows:

Bromo-NN-bis(trimethylsilyl)aniline (3.3 g, 10.43 mmol) in tetrahydrofuran (20 mL) was cooled to −75° C. A 2.5M solution of n-butyllithium in hexane (5 mL, 12.5 mmol) was added to the reaction and allowed to stir at −75° C. for 5 hours. 1-Chloro-4-pentanone (1.63 g, 11.5 mmol) added to cold reaction mixture and once addition was complete, reaction warmed to room temperature and stirred for 120 hours. Reaction heated to 70° C. for 24 hours and reaction was concentrated. To the residue was added methanol (30 mL) and 1M aqueous hydrogen chloride (10 mL) and stirred at 60° C. for 2 hours. Reaction concentrated, neutralized with saturated sodium bicarbonate and extracted with ethyl acetate. The extract was washed with brine, dried over magnesium sulfate, filtered and concentrated. Crude purified on silica gel, eluting with a gradient from 20% to 70% ethyl acetate in heptane to give 4-(2-methyl-tetrahydrofuran-2-yl)benzenamine (510 mg, 27%).

1H NMR (500 MHz, CHLOROFORM-d) δ ppm 1.51 (s, 3H) 1.79-1.87 (m, 1H) 1.93-2.01 (m, 2H) 2.14-2.22 (m, 1H) 3.87-3.93 (m, 1H) 3.93 (s, 2H) 3.97-4.03 (m, 1H) 6.68 (d, 2H) 7.21 (d, 2H)

(9K): 1H NMR (500 MHz, DMSO-d6) δ ppm 1.43 (s, 3H) 1.69-1.77 (m, 1H) 1.90-2.03 (m, 2H) 2.06-2.13 (m, 1H) 2.80 (t, 2H) 3.73 (s, 3H) 3.80 (t, 2H) 3.86-3.94 (m, 2H) 7.22 (d, 2H) 7.37 (d, 2H) 7.79 (d, 1H) 8.37 (d, 1H)

m/z (M+1)=355.1

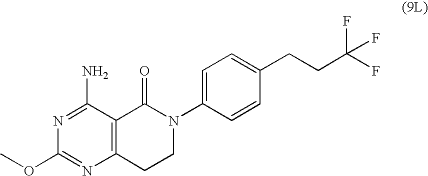

### 4-amino-2-methoxy-6-[4-(3,3,3-trifluoropropyl)phenyl]-7,8-dihydropyrido[4,3-d]pyrimidin-5(6H)-one

Prepared analogous to (9A) from 4-(3,3,3-trifluoropropyl)benzenamine which was prepared as follows:

Trifluoromethanesulfonic acid 2,2,2-trifluoroethyl ester (5.64 g, 24.3 mmol) was dissolved in toluene (30 mL) and triphenylphosphine (9.57 g, 36.5 mmol) was added. The reaction mixture was heated to 100° C. for 48 hours. Brown gum formed. Solvent decanted and the remaining brown gum was dried to give the ylide (8 g, 70%) which was used in the following reaction without further purification.

The ylide (7.85 g, 15.9 mmol) and 4-nitro benzaldehyde (1.6 g, 11 mmol) were combined in dimethylformamide (30 mL). Cesium fluoride (3.74 g, 24.4 mmol) added and reaction stirred at room temperature for 18 hours. Water (200 mL) was added and the reaction mixture was extracted with 1:2 ethyl acetate:heptane (300 mL). Organics dried over magnesium sulfate, filtered and concentrated. Crude purified on silica gel eluting with a gradient from 5% to 20% ethyl acetate in heptane to give (E)-1-nitro-4-(3,3,3-trifluoroprop-1-enyl)benzene (510 mg, 22%) as a brown oil.

GCMS was 217 at 1.72 min and 1.82 min.

(E)-1-nitro-4-(3,3,3-trifluoroprop-1-enyl)benzene (510 mg, 2.35 mmol) was dissolved in ethanol (15 mL) and ethyl acetate (15 mL). 20% Palladium hydroxide on carbon (250 mg) was carefully added to reaction vessel under a steady stream of nitrogen. The reaction was stirred in a parr shaker at room temperature under hydrogen (50 PSI) for 20 hours. The reaction mixture was filtered through a pad of celite and filtrate concentrated. Crude oil was purified on silica gel, eluting with a gradient from 5% to 40% ethyl acetate in heptane to give 4-(3,3,3-trifluoropropyl)benzenamine (210 mg, 47%).

GCMS was 189 at 1.35 min.

1H NMR (400 MHz, CHLOROFORM-d) δ ppm 2.22-2.42 (m, 2H) 2.68-2.80 (m, 2H) 3.57 (br. s., 2H) 6.59-6.66 (m, 2H) 6.91-7.01 (m, 2H)

(9L): 1H NMR (400 MHz, CHLOROFORM-d) δ ppm 2.29-2.47 (m, 2H) 2.82-2.92 (m, 2H) 3.04 (t, J=6.83 Hz, 2H) 3.86-3.97 (m, 5H) 5.53 (br. s., 1H) 7.24 (s, 4H) 8.59 (br. s., 1H)

m/z (M+1)=367.1

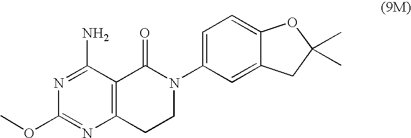

### 4-amino-6-(2,2-dimethyl-2,3-dihydro-1-benzofuran-5-yl)-2-methoxy-7,8-dihydropyrido[4,3-d]pyrimidin-5(6H)-one

Prepared analogous to (9A) from 2,2-dimethyl-2,3-dihydrobenzofuran-5-amine which was prepared as follows:

A suspension of 4-acetamidophenol (40 g, 265 mmol), 3-chloro-2-methyl propene (25.8 mL, 264 mmol) and potassium carbonate (40.8 g, 295 mmol) in dimethylformamide were heated to 100° C. for 6 hours. Reaction cooled to room temperature and poured onto ice water (500 mL) and extracted into ethyl acetate (300 mL). The organic layer was washed with water (2×1 L), dried over magnesium sulfate and concentrated to give N-(4-(2-methylallyloxy)phenyl)acetamide (32.39 g, 60% yield) as a yellow solid.

1H NMR (400 MHz CDCl3) δ ppm 7.36 (d, 2H), 7.01 (s, 1H), 6.86 (d, 2H), 5.04 (d, 2H), 4.40 (s, 3H), 2.15 (s, 3H), 1.81 (s, 3H).

N-(4-(2-methylallyloxy)phenyl)acetamide (26.76 g, 130 mmol) in N,N-diethylaniline (500 mL) was heated to 200° C. for 48 hours. The reaction was allowed to cool to room temperature and 2M hydrochloric acid added. The mixture was extracted with ethyl acetate (3×250 mL) and the combined organics were washed with 2M hydrochloric acid (2×250 mL), dried over magnesium sulfate, filtered and concentrated. The residue was taken up in methanol (400 mL) and cooled with an ice bath. Hydrochloric acid (34%, 150 mL) was added drop wise and the mixture was allowed to warm to room temperature and then heated to reflux overnight. Reaction cooled to room temperature and concentrated. Water was added to the remaining mixture and extracted with ethyl acetate (2×200 mL). The aqueous was basified to pH 5 with 2M sodium hydroxide then neutralized with saturated aqueous sodium bicarbonate. The aqueous was extracted with dichloromethane (3×250 mL), dried over magnesium sulfate, filtered and concentrated to give The combined organics were dried over magnesium sulfate, filtered and concentrated to give 2,2-dimethyl-2,3-dihydrobenzofuran-5-amine (13.31 g, 63%) as a brown oil.

1H NMR (CDCl3, 400 MHz) δ ppm 6.54 (2H, d), 6.44 (1H, dd), 3.38 (2H, br, s), 2.92 (2H, s), 1.44 (6H, s).

(9M): 1H NMR (CDCl3, 400 MHz) δ ppm 8.65 (1H, s), 7.08 (1H, s), 6.98 (1H, d), 6.74 (1H, d), 5.51 (1H, s), 3.95 (3H, s), 3.68 (2H, t), 3.03 (4H, m), 1.48 (6H, s)

m/z (M+1)=341.2

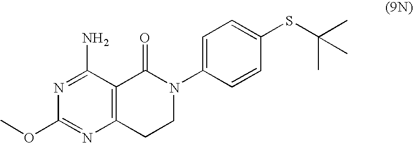

### 4-amino-6-[4-(tert-butylthio)phenyl]-2-methoxy-7,8-dihydropyrido[4,3-d]pyrimidin-5(6H)-one

60% Sodium hydride in mineral oil (850 mg, 21.3 mmol) was added portion wise to dimethylformamide (10 mL) at room temperature and stirred for 20 minutes. Tert-butylthiol was carefully added drop wise at room temperature. Once addition was complete, 1-fluoro-4-nitrobenzene (1.5 g, 10.63 mmol) was carefully added drop wise at room temperature (strong exothermic effect was observed and water bath was used to cool the reaction). Reaction stirred for 30 minutes at room temperature. Water (50 mL) added and stirred for 15 minutes. Solution was extracted with 1:3 ethyl acetate: heptane (2×). Combined organics dried over sodium sulfate, filtered and concentrated. Crude purified on silica gel, eluting with a gradient from 0 to 30% ethyl acetate in heptane to give tert-butyl(4-nitrophenyl)sulfane (2.05 g, 91%) as a slightly orange solid.

1H NMR (400 MHz, CHLOROFORM-d) δ ppm 1.33 (s, 9H) 7.65 (d, 2H) 8.14 (d, 2H)

13C NMR (101 MHz, CHLOROFORM-d) δ ppm 31.31 (s, 3C) 47.75 (s, 1C) 123.53 (s, 2C) 137.07 (s, 2C) 142.53 (s, 1C)

Tert-butyl(4-nitrophenyl)sulfane (2.05 g, 9.702 mmol) in ethanol (70 mL) and 10M hydrochloric acid (2.4 mL). Tin powder (4 g, 33 mmol) was added in one portion at room temperature and the mixture was stirred for 24 hours. 1 M aqueous sodium hydroxide (30 mL) and water (100 mL) added and extracted with ethyl acetate (100 mL). The extract was washed with brine, dried over sodium sulfate, filtered, concentrated and purified on silica, eluting with a gradient from 10% to 40% ethyl acetate in heptane to give 4-(tert-butylthio)benzenamine (1.5 g, 85%).

1H NMR (400 MHz, CHLOROFORM-d) δ ppm 1.23 (s, 9H) 3.89 (s, 2H) 6.62 (d, 2H) 7.29 (d, 2H)

4-(tert-butylthio)benzenamine (660 mg, 3.64 mmol), ethyl propenoate (0.435 mL, 4 mmol), and triethylamine (0.2 mL, 3 mmol) combined in ethanol (0.2 mL) and heated to 100° C. for 24 hours. Reaction mixture concentrated and crude purified on silica gel, eluting with a gradient from 0 to 40% ethyl acetate in heptane to give ethyl 3-(4-(tert-butylthio)phenylamino)propanoate (670 mg, 65%).

1H NMR (400 MHz, CHLOROFORM-d) δ ppm 1.23 (s, 9H) 1.25 (t, 3H) 2.60 (t, 2H) 3.44 (t, 2H) 4.14 (q, 2H) 4.34 (br. s., 1H) 6.55 (d, 2H) 7.30 (d, 2H)

Ethyl 3-(4-(tert-butylthio)phenylamino)propanoate (660 mg, 2.34 mmol), 4-dimethylaminopyridine (28.7 mg, 0.235 mmol) and cyanoacetic acid (239 mg, 2.81 mmol) were combined in dimethylformamide (10 mL) and cooled to 0° C. Diisopropylcarbodiimide (0.436 mL, 2.81 mmol) was added drop wise and once addition was complete, reaction was warmed up to room temperature and stirred for 2 hours. Water (80 mL) was added and reaction was extracted with 1:1 ethyl acetate-heptane. Organic layer was washed with water and brine, dried over magnesium sulfate, filtered and concentrated to give ethyl 3-(N-(4-(tert-butylthio)phenyl)-2-cyanoacetamido)propanoate (817 mg, 100%) which was used in the next step without further purification.

Ethyl 3-(N-(4-(tert-butylthio)phenyl)-2-cyanoacetamido)propanoate (817 mg, 2.34 mmol) was dissolved in methanol (40 mL) and 1,8-diazabicycloundec-7-ene (464 mg, 3.05 mmol) was added the mixture ane heated to 80° C. for 15 minutes. Reaction was cooled, concentrated and dissolved in ethyl acetate (5 mL), heptane (15 mL), and water (15 mL). 1M hydrochloric acid (4 mL) was added and precipitate formed. Mixture was stirred for 30 minutes and solids collected and dried in a vacuum oven at 45° C. to give 1-(4-(tert-butylthio)phenyl)-4-hydroxy-2-oxo-1,2,5,6-tetrahydropyridine-3-carbonitrile (595 mg, 83%) as a white solid.

1H NMR (400 MHz, DMSO-d6) δ ppm 1.21 (s, 9H) 2.79 (t, 2H) 3.80 (t, 2H) 7.27 (d, 2H) 7.44 (d, 2H)

1-(4-(tert-butylthio)phenyl)-4-hydroxy-2-oxo-1,2,5,6-tetrahydropyridine-3-carbonitrile (590 mg, 1.95 mmol) was suspended in dichloromethane (15 mL) and dimethylformaide (0.009 ml, 0.117 mmol). Reaction cooled to 0° C. and oxalyl chloride (1.08 ml, 12.2 mmol) added drop wise. Once addition was complete, reaction was slowly warmed to room temperature and stirred for 24 hours. Reaction was concentrated and toluene (25 mL) added and concentrated to dryness. Methanol (15 mL) was added and stirred at 65° for 18 hours and then at 55° C. for 72 hours. Reaction concentrated to give 1-(4-(tert-butylthio)phenyl)-4-methoxy-2-oxo-1,2,5,6-tetrahydropyridine-3-carbonitrile (617 mg, 100%) which was used in the next step without further purification.

1H NMR (400 MHz, DMSO-d6) δ ppm 1.21 (s, 9H) 3.03 (t, 2H) 3.87 (t, 2H) 4.01 (s, 3H) 7.30 (d, 2H) 7.45 (d, 2H)

1-(4-(tert-butylthio)phenyl)-4-methoxy-2-oxo-1,2,5,6-tetrahydropyridine-3-carbonitrile (617 mg, 1.95 mmol) dissolved in methanol (10 mL) and cyanamide (257 mg, 4.29 mmol) and sodium methoxide (347 mg, 6.43 mmol) were added in one portion at room temperature and stirred for 2 hours. 1 M aqueous solution of potassium hydrogen sulfate (7 mL) was added and reaction concentrated. Water (5 mL) was added and the solids filtered off and dried under vacuum. Solids were then dissolved in methanol (10 mL) and 96% sulfuric acid (0.27 mL) and heated to 70° C. for 20 hours. Solid sodium bicarbonate (1 g) was and stirred at 70° C. for 15 minutes. Tetrahydrofuran (20 mL) was added, cooled to room temperature and solids filtered off. Filtrate was concentrated and purified on silica gel, eluting with a gradient from 40 to 90% ethyl acetate in heptane to give the target compound, (9N) (150 mg, 21%).

1H NMR (500 MHz, CHLOROFORM-d) δ ppm 1.31 (s, 9H) 3.07 (t, 2H) 3.97 (t, 2H) 3.96 (s, 3H) 5.68 (s, 1H) 7.31 (d, 2H) 7.58 (d, 2H) 8.61 (d, 1H)

m/z (M+1)=359.1

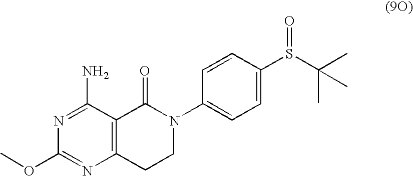

### 4-amino-6-[4-(tert-butylsulfinyl)phenyl]-2-methoxy-7,8-dihydropyrido[4,3-d]pyrimidin-5(6H)-one

(9N) (22 mg, 0.061 mmol) in dichloromethane (4 mL) was cooled to −78° C. and 3-chloroperoxybenzoic acid (21 mg, 0.095 mmol) was added in one portion and continued to stir at −78° C. for 1.5 hours. Saturated aqueous sodium sulfite (3 mL) was added and the reaction mixture was slowly warmed to room temperature. Ethyl acetate (5 ml) and saturated aqueous sodium bicarbonate (3 mL) were added, and the mixture was stirred at room temperature for 30 minutes. The mixture was extracted with ethyl acetate (10 mL), was washed with brine, dried over magnesium sulfate, filtered, concentrated and purified on silica gel, eluting with a gradient from 4 to 10% methanol in ethyl acetate to give the target compound (9O) (10 mg, 44%).

1H NMR (500 MHz, CHLOROFORM-d) δ ppm 1.22 (s, 9H) 3.13 (t, 2H) 3.99 (s, 3H) 4.02 (t, 2H) 5.72 (s, 1H) 7.49 (d, 2H) 7.66 (d, 2H) 8.62 (s, 1H)

m/z (M+1)=375.1

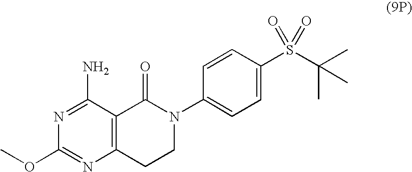

### 4-amino-6-[4-(tert-butylsulfonyl)phenyl]-2-methoxy-7,8-dihydropyrido[4,3-d]pyrimidin-5(6H)-one

(9N) (30 mg, 0.084 mmol) in dichloromethane (4 mL) was cooled to −78° C. and 3-chloroperoxybenzoic acid (57 mg, 0.26 mmol) was added in one portion and continued to stir at −78° C. for 3 hours. Saturated aqueous sodium sulfite (3 mL) was added and the reaction mixture was slowly warmed to room temperature. Ethyl acetate (5 ml) and saturated aqueous sodium bicarbonate (3 mL) were added, and the mixture was stirred at room temperature for 30 min. The mixture was extracted with ethyl acetate (10 mL), was washed with brine, dried over magnesium sulfate, filtered, concentrated and purified on silica gel, eluting with a gradient from 0% to 5% methanol in ethyl acetate to give the target compound (9P) (23 mg, 70%).

1H NMR (500 MHz, CHLOROFORM-d) δ ppm 1.38 (s, 9H) 3.12 (t, 2H) 3.99 (s, 3H) 4.04 (t, 2H) 5.76 (s, 1H) 7.55 (d, 2H) 7.92 (d, 2H) 8.56 (s, 1H)

m/z (+1)=391.1

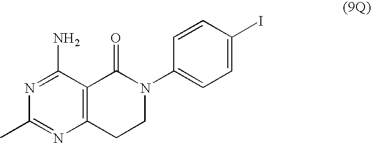

### 4-amino-6-(4-iodophenyl)-2-methyl-7,8-dihydropyrido[4,3-d]pyrimidin-5(6H)-one

Prepared analogous to example (9A) from 4-iodoaniline.

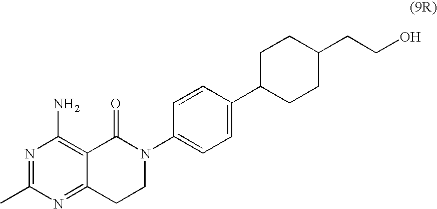

### 4-amino-6-{4-[4-(2-hydroxyethyl)cyclohexyl]phenyl}-2-methyl-7,8-dihydropyrido[4,3-d]pyrimidin-5(6H)-one

(9Q) and methyl 2-(4-(4,4,5,5-tetramethyl-1,3,2-dioxaborolan-2-yl)cyclohex-3-enyl)acetate (73.7 mg, 0.263 mmol) were combined in tetrahydrofuran (2 mL). Palladium (0) tetrakis(triphenylphosphine) (13 mg, 0.011 mmol) and cesium carbonate (105 mg, 0.316 mmol) added and reaction heated to reflux for 16 hours. The reaction mixture was cooled to room temperature, diluted with ethyl acetate, washed with water and brine, dried over magnesium sulfate and concentrated. The crude material was purified on silica gel eluting with a gradient from 2% to 5% methanol in dichloromethane to give methyl 2-(4-(4-(4-amino-2-methyl-5-oxo-7,8-dihydropyrido[4,3-d]pyrimidin-6(5H)-yl)phenyl)cyclohex-3-enyl)acetate (82 mg, 77%) as a yellow solid.

m/z (M+1)=407.4

Methyl 2-(4-(4-(4-amino-2-methyl-5-oxo-7,8-dihydropyrido[4,3-d]pyrimidin-6(5H)-yl)phenyl)cyclohex-3-enyl)acetate (82 mg, 0.2 mmol) was dissolved in ethanol (10 mL) and ethyl acetate(10 mL). Under a steady stream of hydrogen, 20% palladium hydroxide on carbon (40 mg) was added. The reaction mixture was placed in a parr shaker and set up to run at room temperature under 50 PSI of hydrogen for 16 hours. Reaction filtered through a pad of celite under a nitrogen atmosphere. Filtrated concentrated to give methyl 2-(4-(4-(4-amino-2-methyl-5-oxo-7,8-dihydropyrido[4,3-d]pyrimidin-6(5H)-yl)phenyl)cyclohexyl)acetate (80 mg, 97%) as an off-white solid.

Methyl 2-(4-(4-(4-amino-2-methyl-5-oxo-7,8-dihydropyrido[4,3-d]pyrimidin-6(5H)-yl)phenyl)cyclohexyl)acetate (35 mg, 0.086 mmol) dissolved in tetrahydrofuran (2 mL) and cooled to 0° C. A 1 M solution of diisobutylaluminium hydride in dichloromethane was added drop wise to the reaction solution. Stirred for 30 minutes at 0° C. and stirred at room temperature for 4 hours. Ethyl acetate (10 ml) and 1M Rochelle salt solution (10 mL) were added and the mixture was stirred until solution turned clear. Organic layer was separated and dried over magnesium sulfate, filtered and concentrated. Crude purified on silica gel column eluting with a gradient from 2% to 6% methanol in dichloromethane to give the target compound (9R) (15 mg, 46%) as a white solid.

1H NMR (400 MHz, METHANOL-d4) δ ppm 1.05-1.19 (m, 1H) 1.41-1.57 (m, 2H) 1.60-1.75 (m, 5H) 1.81-1.94 (m, 3H) 2.42 (s, 3H) 2.46-2.63 (m, 1H) 3.04 (t, J=6.83 Hz, 2H) 3.57-3.65 (m, 2H) 3.87-3.97 (m, 2H) 7.21-7.34 (m, 4H)

m/z (M+1)=381.4

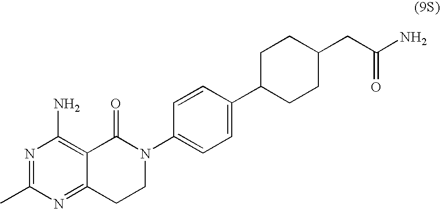

### 2-{4-[4-(4-amino-2-methyl-5-oxo-7,8-dihydropyrido[4,3-d]pyrimidin-6(5H)-yl)phenyl]cyclohexyl}acetamide

Methyl 2-(4-(4-(4-amino-2-methyl-5-oxo-7,8-dihydropyrido[4,3-d]pyrimidin-6(5H)-yl)phenyl)cyclohexyl)acetate (210 mg, 0.514 mmol) was dissolved in a solution of tetrahydrofuran (12 mL), methanol (3 mL) and water (2.1 mL). Lithium hydroxide (49.2 mg, 2.06 mmol) was added and reaction solution heated to 60° C. for 3 hours. 1M aqueous hydrochloric acid was added to reaction solution to adjust PH to about 3 and reaction mixture was extracted with 20% iso-propanol in dichloromethane. The organic layer was dried over magnesium sulfate, filtered and concentrated to give 2-(4-(4-(4-amino-2-methyl-5-oxo-7,8-dihydropyrido[4,3-d]pyrimidin-6(5H)-yl)phenyl)cyclohexyl)acetic acid (150 mg, 74%) as a white solid.

m/z (M+1)=395.3

2-(4-(4-(4-amino-2-methyl-5-oxo-7,8-dihydropyrido[4,3-d]pyrimidin-6(5H)-yl)phenyl)cyclohexyl)acetic acid (100 mg, 0.254 mmol) was dissolved in 1,2-dichloroethane and cooled to 0° C. Oxalyl chloride (10 eq.) was added drop wise and once addition was complete, the reaction mixture was warmed up to room temperature for 3 hours. Reaction was concentrated and 1,2-dichoroethane was added and concentrated (2×). A 0.5M solution of ammonia in 1,4-dioxane was added to the concentrate and stirred at room temperature for 16 hours. Methanol (2 ml), ethyl acetate (30 mL) and water were added. The organic layer was washed with saturated aqueous solution of sodium bicarbonate and brine and dried over magnesium sulfate, filtered and concentrated. Crude purified on silica gel eluting with a gradient from 2% to 10% methanol in dichloromethane to the target compound (9S) (50 mg, 50%) as a white solid.

m/z (M+1)=394.5

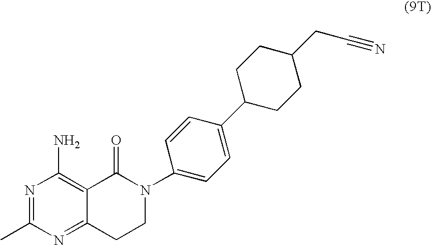

### {4-[4-(4-amino-2-methyl-5-oxo-7,8-dihydropyrido[4,3-d]pyrimidin-6(5H)-yl)phenyl]cyclohexyl}acetonitrile

2-{4-[4-(4-amino-2-methyl-5-oxo-7,8-dihydropyrido[4,3-d]pyrimidin-6(5H)-yl)phenyl]cyclohexyl}acetamide (40 mg, 0.1 mmol) in tetrahydrofuran (1 mL) and dimethylformamide (0.008 mL, 0.102 mmol). Oxalyl chloride (0.04 mL, 0.5 mmol) was added at room temperature and stirred for 2 hours. Saturation aqueous sodium bicarbonate was carefully added to the reaction mixture and concentrated to get rid of organic solvents. Reaction was extracted with ethyl acetate and combined organics washed with water, dried over magnesium sulfate and concentrated. Crude purified on silica gel eluting with a gradient from 1% to 5% methanol in dichloromethane to give the target compound (9T) (5 mg, 10%) as a white solid.

1H NMR (400 MHz, CHLOROFORM-d) δ ppm 1.20-1.35 (m, 1H) 1.40-1.56 (m, 1H) 1.62 (br. s., 2H) 1.69-1.83 (m, 2H) 1.89-2.04 (m, 2H) 2.31 (d, J=6.64 Hz, 2H) 2.44 (d, J=7.81 Hz, 1H) 2.46-2.56 (m, 4H) 3.07 (t, J=7.42 Hz, 2H) 3.90-4.00 (m, 2H) 5.56 (br.s., 1H) 7.20-7.28 (m, 4H) 8.52 (br.s., 1H)

m/z (M+1)=376.4

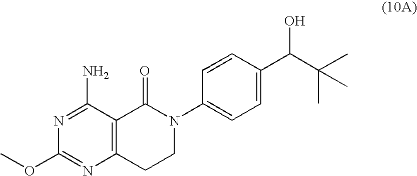

### 4-amino-6-[4-(1-hydroxy-2,2-dimethylpropyl)phenyl]-2-methoxy-7,8-dihydropyrido[4,3-d]pyrimidin-5(6H)-one

t-butyl magnesium chloride in tetrahydrofuran (9 mL, 8.11 mmol) was added to a solution of 4-bromo benzaldehyde (1 g, 5.4 mmol) in tetrahydrofuran (20 mL) at 0° C. Once addition was complete, reaction warmed up to room temperature and stirred for 18 hours. Saturated aqueous ammonium chloride (10 mL) added and extracted with ethyl acetate. The organic layer was separated and dried over magnesium sulfate and concentrated. Crude purified on silica gel eluting with a gradient from 3% to 10% ethyl acetate in heptane to give 1-(4-bromophenyl)-2,2-dimethylpropan-1-ol (630 mg, 47%) as a colorless oil.

1H NMR (400 MHz, CHLOROFORM-d) δ ppm 0.89 (s, 9H) 1.85 (s, 1H) 4.34 (s, 1H) 7.16 (d, J=8.20 Hz, 2H) 7.42 (d, J=8.39 Hz, 2H)

To a solution of 1-(4-bromophenyl)-2,2-dimethylpropan-1-ol (630 mg, 2.59 mmol) in dimethylformamide at room temperature under nitrogen was added tert-butyldimethylsilyl chloride (818 mg, 5.43 mmol) and stirred for 65 hours. Reaction mixture was concentrated, diluted with water (50 mL) and extracted with 1:1 ethyl acetate and heptane (100 mL). The organic phase was separated, washed with brine, dried over magnesium sulfate, and concentrated to give (1-(4-bromophenyl)-2,2-dimethylpropoxy)(tert-butyl)dimethylsilane (900 mg, 97%) as a colorless oil. Used without further purification in the following step.

Aminoester hydrochloride (310 mg, 2.02 mmol), (1-(4-bromophenyl)-2,2-dimethylpropoxy)(tert-butyl)dimethylsilane (600 mg, 1.68 mmol), cesium carbonate (1.09 g, 3.36 mmol) and diisopropylethylamine (0.3 mL, 2 mmol) were combined in degassed toluene (200 mL). X-Phos (40 mg, 0.08 mmol) and palladium acetate (18.9 mg, 0.084 mmol) were added and the reaction was bubbled with nitrogen for an additional 5 minutes. The reaction mixture was heated to reflux for 20 hours. The reaction mixture was cooled to room temperature and filtered through a pad of celite using ethyl acetate to wash. Filtrate concentrated and purified on silica gel eluting with a gradient from 3% to 15% ethyl actetate in heptane to give ethyl 3-(4-(1-(tert-butyldimethylsilyloxy)-2,2-dimethylpropyl)phenylamino)propanoate (150 mg, 22%) as a yellow oil.

1H NMR (400 MHz, CHLOROFORM-d) δ ppm −0.34 (s, 3H) −0.03 (s, 3H) 0.82 (s, 9H) 0.87 (s, 9H) 1.20-1.28 (m, 3H) 2.60 (t, J=6.34 Hz, 2H) 3.42 (t, J=6.34 Hz, 2H) 4.01 (br. s., 1H) 4.10-4.19 (m, 2H) 6.52 (d, J=8.59 Hz, 2H) 7.02 (d, J=8.39 Hz, 2H)

To a suspension of cyanoacetic acid (64.8 mg, 0.762 mmol) and dimethylformamide (0.6 uL) in dichloromethane (2 mL) was added oxalyl chloride (0.06 mL) at room temperature and the reaction mixture was stirred for 40 minutes. To this was then added ethyl 3-(4-(1-(tert-butyldimethylsilyloxy)-2,2-dimethylpropyl)phenylamino)propanoate in dichloromethane and the reaction cooled to 0° C. Triethylamine (0.106 mL, 0.762 mmol) was added and reaction mixture slowly allowed to warm up to room temperature and stirred for 18 hours. Reaction was washed with saturated aqueous sodium bicarbonate (10 mL), dried over magnesium sulfate, concentrated and purified on silica gel eluting with a gradient from 5% to 25% ethyl acetate in heptane to give ethyl 3-(N-(4-(1-(tert-butyldimethylsilyloxy)-2,2-dimethylpropyl)phenyl)-2-cyanoacetamido)propanoate (100 mg, 57%) as a brown oil.

m/z (M+1)=461.2

Ethyl 3-(N-(4-(1-(tert-butyldimethylsilyloxy)-2,2-dimethylpropyl)phenyl)-2-cyanoacetamido)propanoate (100 mg, 0.217 mmol) and 1,8-diazabicyclo[5.4.0]undec-7-ene (92 mg, 0.6 mmol) in methanol (2 mL) was heated to 90° C. for 1 hour. Reaction mixture was concentrated and diluted with water (25 mL), ethyl acetate (5 mL), heptane (10 mL) and 1 ml of 1 M hydrochloric acid. Precipitate formed and reaction solution was filtered to give 1-(4-(1-(tert-butyldimethylsilyloxy)-2,2-dimethylpropyl)phenyl)-4-hydroxy-2-oxo-1,2,5,6-tetrahydropyridine-3-carbonitrile (20 mg, 22%) as a white solid.

1-(4-(1-(tert-butyldimethylsilyloxy)-2,2-dimethylpropyl)phenyl)-4-hydroxy-2-oxo-1,2,5,6-tetrahydropyridine-3-carbonitrile (800 mg, 1.93 mmol) in dimethylformamide (0.015 mL) and dichloromethane (20 mL) was cooled to 0° C. Oxalyl chloride (0.668 mL, 7.72 mmol) was added drop wise and the mixture was warmed to room temperature and stirred for 1 hour. The mixture was concentrated to dryness and toluene (5 mL) added and reaction mixture concentrated to dryness. Methanol (10 mL) was then added and stirred at 55° C. for 18 hours. Reaction mixture was then concentrated and purified on silica gel eluting with a gradient from 30% to 70% ethyl acetate in heptane to give 4-methoxy-1-(4-(1-methoxy-2,2-dimethylpropyl)phenyl)-2-oxo-1,2,5,6-tetrahydropyridine-3-carbonitrile (10B) (300 mg, 49%) as a white solid.

1H NMR (500 MHz, METHANOL-d4) δ ppm 0.89 (s, 9H) 2.97 (t, J=6.83 Hz, 2H) 3.88-3.94 (m, 2H) 4.14 (s, 3H) 4.33 (s, 1H) 7.18 (d, J=8.29 Hz, 2H) 7.32 (d, J=8.29 Hz, 2H)

1-(4-(1-hydroxy-2,2-dimethylpropyl)phenyl)-4-methoxy-2-oxo-1,2,5,6-tetrahydropyridine-3-carbonitrile (10C) (250 mg, 39%) was also isolated.

1H NMR (500 MHz, CHLOROFORM-d) δ ppm 0.89 (s, 9H) 2.86 (t, J=6.71 Hz, 2H) 3.19 (s, 3H) 3.77 (s, 1H) 3.92 (t, J=6.83 Hz, 2H) 4.18 (s, 3H) 7.25 (d, J=2.93 Hz, 4H)

m/z (M+1)=329.1

(10C) (130 mg, 0.414 mmol) was dissolved in methanol (2 mL). Methyl isourea (91.5 mg, 0.828 mmol) and diisopropylethylamine (0.361 mL, 2.07 mmol) were added and heated to 80° C. for 1 hour. The mixture was concentrated and dissolved in ethyl acetate and washed with water, dried over magnesium sulfate, concentrated and purified on silica gel eluting with a gradient from 40% to 70% ethyl acetate in heptane to give the target compound, (10A) (78 mg, 53%) as a white solid

1H NMR (400 MHz, CHLOROFORM-d) δ ppm 0.93 (s, 9H) 1.90 (d, J=2.54 Hz, 1H) 3.05 (t, J=6.83 Hz, 2H) 3.88-3.97 (m, 5H) 4.36-4.45 (m, 1H) 5.59 (br. s., 1H) 7.25 (d, J=8.59 Hz, 2H) 7.31-7.41 (m, 2H) 8.61 (br. s., 1H)

m/z (M+1)=357.1

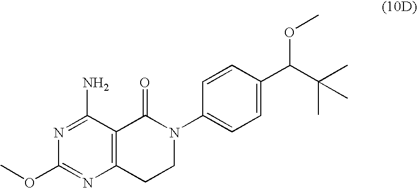

### 4-amino-2-methoxy-6-[4-(1-methoxy-2,2-dimethylpropyl)phenyl]-7,8-dihydropyrido[4,3-d]pyrimidin-5(6H)-one

To a solution of cyanamide (52.6 mg, 0.875 mmol) in methanol (5 mL) was added sodium methoxide (65 mg, 1.14 mmol), and stirred for 15 minutes at room temperature. The solution was added to a suspension of (10B) (250 mg, 0.761 mmol) in methanol (5 mL) and the mixture stirred for 1 hour at room temperature. Sulfuric acid (0.21 mL, 3.8 mmol) was added and allowed to stir at 55° C. for 16 hours. Reaction was concentrated and water (10 mL) added. Reaction was then made basic with aqueous 1N sodium hydroxide. Reaction mixture was then diluted with ethyl acetate and layers separated. The organic layer was then dried over magnesium sulfate, filtered, concentrated and purified on silica gel eluting with a gradient from 15 to 5% methanol in dichloromethane to give the target compound, (10D) (120 mg, 42%) as a white solid

1H NMR (500 MHz, CHLOROFORM-d) δ ppm 0.91 (s, 9H) 3.09 (t, J=6.83 Hz, 2H) 3.22 (s, 3H) 3.80 (s, 1H) 3.99 (t, J=6.71 Hz, 5H) 5.59 (br. s., 1H) 7.24-7.36 (m, 4H) 8.68 (br. s., 1H)

m/z (M+1)=371.1

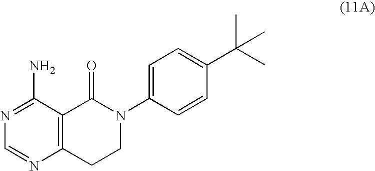

### 4-amino-6-(4-tert-butylphenyl)-7,8-dihydropyrido[4,3-d]pyrimidin-5(6H)-one

Sodium hydride (0.40 g, 0.016 mol) and cyanamide (0.44 g, 10.6 mmol) were added to dioxane (20 mL) and the mixture stirred for 10 minutes. The mixture was added drop wise to (6A-1) (2.0 g, 7.04 mmol) in dioxane (100 mL) and the resulting mixture stirred at room temperature for 4 hours. Hydrochloric acid in dioxane (4N, 10 mL) was added and the mixture heated to reflux overnight. Reaction concentrated and the crude product was purified by flash column chromatography eluting with 66% ethyl acetate in hexane to give 4-amino-6-(4-tert-butylphenyl)-2-chloro-7,8-dihydropyrido[4,3-d]pyrimidin-5(6H)-one (1.51 g, 65%).

1H NMR (400 MHz, CDCl3): δ ppm 8.77 (bs, 1H), 7.43 (d, 2H), 7.23 (d, 2H), 5.97 (bs, 1H), 3.98 (t, 2H), 3.11 (t, 2H), 1.32 (s, 9H)

4-Amino-6-(4-tert-butylphenyl)-2-chloro-7,8-dihydropyrido[4,3-d]pyrimidin-5(6H)-one (1.10 g, 3.3 mmol) was dissolved in ethyl acetate (40 mL) and triethylamine (1.0 mL). Palladium hydroxide was added and the mixture stirred under 10 Torr hydrogen for 20 hours. The mixture was then filtered through celite and filtrate concentrated. The crude product was purified on silica gel eluting with 50% ethyl acetate in hexane to give the target compound (11A) (0.30 g, 31%).

1H NMR (400 MHz, d-MeOH): δ ppm 8.34 (s, 1H), 7.48 (d, 2H), 7.28 (d, 2H), 3.95 (t, 2H), 3.08 (t, 2H), 1.32 (s, 9H)

m/z (M+1)=297.2

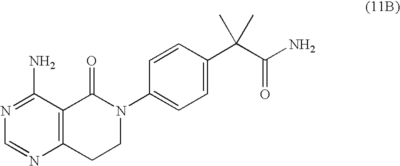

### 2-[4-(4-amino-5-oxo-7,8-dihydropyrido[4,3-d]pyrimidin-6(5H)-yl)phenyl]-2-methylpropanamide

XPhos (1.5 g, 3.1 mmol) and palladium acetate were added to degassed toluene (600 mL) and stirred for 15 minutes at room temperature. Cesium carbonate (60 g, 185 mmol), diisopropylethyl amine (21.6 mL), (3A-1) (16 g, 62 mmol) and beta-alanine ethyl ester (19 g, 124 mmol) were added and the mixture stirred at reflux for 6 hours. The crude reaction mixture was filtered through a pad of celite, washing with toluene. The filtrate was concentrated to afford a yellow oil, which was dissolved in ethyl acetate (300 mL) and washed with 1M hydrochloric acid (500 mL). The organic layer was dried over magnesium sulfate, filtered and concentrated to give (11B-1).

1H NMR (400 MHz CDCl3) δ ppm 7.14 (d, 2H), 6.58 (d, 2H), 4.13-4015 (m, 2H), 3.62 (s, 3H), 3.42 (q, 2H), 2.60 (t, 2H), 1.52 (s, 6H), 1.23-1.25 (m, 3H)

A solution of (11B-1) (18 g, 61 mmol), cyanoacetic acid (10.4 g, 122 mmol), dimethylaminopyridine (7.4 g, 61 mmol) and 1-ethyl-3-(3-dimethylaminopropyl)carbodiimide (16.4 g, 85.4 mmol) in dichloromethane (700 mL) were stirred at room temperature for 60 hours. Reaction concentrated and purified on silica gel eluting with 20% ethyl acetate in hexanes to give (11B-2) (10.99 g, 52% yield).

1H NMR (400 MHz CDCl3) δ ppm 7.42 (d, 2H), 7.18 (d, 2H), 3.99-4.07 (m, 4H), 3.68 (s, 3H), 3.18 (s, 2H), 2.58 (t, 2H), 1.59 (s, 6H), 1.18 (t, 3H)

(11B-2) (10.99 g, 30.8 mmol) was added to degassed methanol (60 mL) and 1,8-diazabicycloundec-7-ene (5.6 mL) was added and heated to reflux for 4 hours. Reaction was concentrated and poured onto aqueous citric acid (50 mL). The product was extracted into ethyl acetate (2×200 mL), dried over magnesium sulfate and concentrated to give methyl 2-(4-(3-cyano-4-hydroxy-2-oxo-5,6-dihydropyridin-1(2H)-yl)phenyl)-2-methylpropanoate (11B-3) (6.3 g, 65%) as a light yellow solid.

1H NMR (400 MHz MeOD) δ ppm 7.34 (d, 2H), 7.25 (d, 2H), 3.80-3.90 (m, 2H), 3.62 (s, 3H), 2.80-2.87 (m, 2H), 1.54 (s, 3H)

(11B-3) (1.86 g, 5.9 mmol) was dissolved in dichloromethane (50 mL) and cooled to 0° C. Oxalyl chloride (1.5 mL, 17.8 mmol) was added followed by the drop wise addition of dimethylformamide (200 uL). Once addition was complete, reaction was warmed up to room temperature and stirred for 1 hour. Reaction was concentrated and toluene was added to the reaction residue and concentrated to dryness. The residue was then dissolved in methanol and heated to reflux for 5 hours. Reaction cooled to room temperature and solids precipitated out of solution. Solids were collected to give methyl 2-(4-(3-cyano-4-methoxy-2-oxo-5,6-dihydropyridin-1(2H)-yl)phenyl)-2-methylpropanoate (11B-4) (811 mg, 47% yield).

1H NMR (400 MHz CDCl3) δ ppm 7.33 (d, 2H), 7.23 (d, 2H), 4.21 (s, 3H), 3.85-3.88 (m, 2H), 3.64 (s, 3H), 2.80 (t, 2H), 1.53 (s, 6H)

(11B-4) (811 mg, 2.5 mmol), cyanamide (240 mg, 5.57 mmol) and sodium methoxide (400 mg, 7.5 mmol) in methanol (70 mL) was stirred at room temperature for two hours. Reaction concentrated and dioxane (70 mL) and hydrochloric acid in dioxane (4M, 6 mL) were added and stirred for 16 hours. Water (250 mL) was added and reaction extracted with ethyl acetate. The organic layer was washed with brine (200 mL), dried over magnesium sulfate and concentrated to give methyl 2-(4-(4-amino-2-chloro-5-oxo-7,8-dihydropyrido[4,3-d]pyrimidin-6(5H)-yl)phenyl)-2-methylpropanoate (11B-5) (900 mg, 96% yield).

1H NMR (300 MHz CDCl3) δ ppm 8.74 (s, 1H), 7.39 (d, 2H), 7.26 (d, 2H), 5.91 (s, 1H), 3.95-3.96 (m, 2H), 3.65 (s, 3H), 3.10 (t, 2H), 1.57 (s, 6H).

Palladium hydroxide (350 mg) was added to a stirred solution of (11-B5) (900 mg, 2.6 mmol) and ammonium formate (1.3 g) in methanol (8 mL) and ethyl acetate (18 mL). The solution was stirred at reflux for 4 hours. Reaction mixture was filtered though a pad of celite washing with methanol. The filtrate was concentrated to give methyl 2-(4-(4-amino-5-oxo-7,8-dihydropyrido[4,3-d]pyrimidin-6(5H)-yl)phenyl)-2-methylpropanoate (11-B6) (667 mg, 75% yield).

1H NMR (400 MHz CDCl3) δ ppm 8.56 (s, 1H), 8.48 (s, 1H), 7.39 (d, 2H), 7.29 (d, 2H), 5.66 (s, 1H), 3.96-4.00 (m, 2H), 3.65 (s, 3H), 3.14 (t, 2H), 1.58 (s, 6H)

Lithium hydroxide (784 mg) in water (8 mL) was added to a stirred solution of (11-B6) (667 mg, 2 mmol) in dioxane (25 mL). The solution was heated to 45° C. and stirred for 16 hours. The pH of the reaction mixture was adjusted to 3 by the addition of 10% aqueous citric acid. A precipitate was formed, which was collected by filtration and washed with water, methanol and dichloromethane to give 2-(4-(4-amino-5-oxo-7,8-dihydropyrido[4,3-d]pyrimidin-6(5H)-yl)phenyl)-2-methylpropanoic acid (11B-7) (451 mg, 74% yield) as a yellow solid.

1H NMR (400 MHz DMSO-D6) δ ppm 8.35 (S, 1H), 8.25 (s, 1H), 7.79 (s, 1H), 7.34 (d, 2H), 7.30 (d, 2H), 3.89 (t, 2H), 2.98 (t, 2H), 1.45 (s, 6H)

3-[cyano(ethyl)amino]propyl-dimethylazanium chloride (344 mg, 1.8 mmol) and O-benzotriazole-N,N,N′,N′-tetramethyl-uronium-hexafluoro-phosphate (343 mg, 2.23 mmol) were added to a stirred suspension of (11-B7) (451 mg, 1.4 mmol) in dimethylformamide (4.2 mL). After stirring for 30 minutes, ammonium hydroxide (3 mL) was added and the suspension stirred for one week. The mixture was concentrated and a further portion of 3-[cyano(ethyl)amino]propyl-dimethylazanium chloride (344 mg) and O-benzotriazole-N,N,N′,N′-tetramethyl-uronium-hexafluoro-phosphate (343 mg) added and the mixture stirred for 1 hour. Ammonia (3 mL) was then added and the reaction mixture stirred for 4 hours. The reaction mixture was filtered before the solid being washed with water (50 mL) then hexane (50 mL). The crude product was re-crystallized in methanol to afford the target compound (11B) (101 mg, 22% yield) as an off white solid.

1H NMR (400 MHz DMSO-D6) δ ppm 8.35 (s, 1H), 8.24 (s, 1H), 7.78 (s, 1H), 7.34 (d, 2H), 7.26 (d, 2H), 6.90 (d, 2H), 3.89 (t, 2H), 2.96 (t, 2H), 1.41 (s, 6H)

m/z (M+1)=326.3

### Pharmacological Testing

The practice of the invention for the treatment of diseases modulated by the inhibition of DGAT-1 can be evidenced by activity in at least one of the protocols described hereinbelow.

### In Vitro Assay for Inhibition of DGAT-1 Activity

Human full-length diacylglycerol:acylCoA acyltransferase 1 (DGAT-1) was expressed in Sf9 insect cells which are then lysed and a crude membrane fraction (105,000×g pellet) was prepared. The DGAT-1 gene is a human DGAT-1 gene described in J Biol Chem 273:26765 (1998) and U.S. Pat. No. 6,100,077.

In vitro inhibition of DGAT-1 was measured using a modification, further described below, of the assay methodology described in U.S. Pat. No. 6,994,956 B2.

The cells were cultured as follows. Sf9 cells (20 L) were infected with 4 mL of DGAT-1 Baculovirus Infected Insect Cells (BIIC) for 72 hours in a Wave Bioreactor System 20/50P (Wave Biotec/GE Healthcare).

Crude DGAT-1 microsomes were prepared as follows. Cell pellets were washed once with ice-cold Dulbecco's phosphate-buffered saline. Cells were collected in tabletop centrifuge (Beckman GS-6KR), 15 minutes, 2000×g, 4° C. Twenty (20) mL of ice-cold Microsome Buffer (MB) was added per 5 g of cell pellet. The suspension was passed through a microfluidizer 3 times (18K psi). The lysate was transferred to centrifuge tubes and centrifuged for 20 minutes at 5000×g (Beckman-Coulter, Inc. Allegra® 64R High-Speed Refrigerated Benchtop Centrifuge, F0650 rotor) at 4° C. The supernatant was transferred to ultracentrifuge tubes and centrifuged at 125,000×g for 1 hour in a Beckman Ti-45 rotor, 4° C. The supernatant fluid was discarded. The pellet was resuspended in 70 mL of MB by sonication. The microsome concentration was determined using Bio-Rad Protein DC Protein Assay. The microsomes were filter sterilized with a 0.22 μm filter. The samples were portioned, flash frozen and stored at −80° C.

The Microsome Buffer, used for microsome preparation, was prepared by conventional means and contained 125 mM sucrose, 3 mM imidazole, 0.2 μg/mL aprotinin, 0.2 μg/mL leupeptin and 5 mM dithiothreitol (Cleland's reagent), DGAT-1 activity was measured in 384-well format in a total assay volume of 25 μl that contained, Hepes buffer (50 mM, pH 7.5), MgCl₂ (10 mM), bovine serum albumin (0.6 mg/ml), [¹⁴C]decanoylCoA (20 μM, 58 Ci/mol) and membranes (25 μg/ml) into which 1,2 dioleoyl-sn-glycerol (75 μM) in acetone has already been incorporated. Inhibitors in DMSO were pre-incubated with membranes before initiating the DGAT-1 reaction by the addition of decanoylCoA. Two control DGAT-1 reactions were also incubated in parallel: 1) DMSO without inhibitor to measure zero percent effect of inhibition and 2) and a maximally inhibited DGAT-1 reaction (“blank”) incubated with 1 μM {trans-4-[4-(4-amino-2,7,7-trimethyl-7H-pyrimido[4,5-b][1,4]oxazin-6-yl)phenyl]cyclohexyl}acetic acid (WO2004/047755). The DMSO concentration was 2.5%. The inhibitors were present at a range of eight concentrations to generate an apparent IC₅₀ for each compound. The eight inhibitor concentration employed ranged from 10 μM to 3 nM (from high to low concentration). Specifically, the eight concentrations used were 10 μM, 3 μM, 1 μM, 300 nM, 100 nM, 30 nM, 10 nM and 3 nM.

The reactions were allowed to proceed for 1.5 hours at room temperature and then terminated by the addition of 10 μl of HCl (0.5 M), (or in the case of G7829E Human IC50 test, 5 μl of 1.4% phosphoric acid in leu of HCl). Reaction mixtures were neutralized by the addition of 15 μl of tris(hydroxy-methyl)aminomethane (1M, pH 8.0) and then mixed by trituration with 37.5 μl of Microscint™-E (Perkin Elmer). Plates contents were allowed to partition for 15 to 30 min before ¹⁴C was measured in a scintillation spectrometer (Wallac Microbeta Trilux 1450-030 liquid scintillation counter 12 detector in the top-count DPM mode). Percent inhibition of test compounds was computed as 100-((DPM DMSO uninhibited-DPM test compound)/(DPM DMSO uninhibited)).

Four separate assays were performed using 4 different methods of analysis. The method of analysis of Assay 1 was the same as Assay 4 (described above) except microsomes were utilized at 25 μg/mL instead of 5 μg/mL. The method of analysis of Assay 2 was the same as Assay 4 (described above) except eleven (11) concentrations of inhibitor were employed instead of eight (8). The method of analysis of Assay 3 was the same as Assay 2 except the compounds were serially diluted in a different laboratory.

The exemplified compounds of the invention were tested for in vitro DGAT-1 inhibition, and were found to exhibit DGAT-1 inhibition with IC₅₀ values set forth in Table 1. Where this DGAT-1 inhibition assay was performed on a compound more than once, an average is provided for that compound.

TABLE 1
DGAT-1 Reduced Microsome Multidose Assay Results

| | | G7329B | G7329D | G7329E | G7329F | G7829E |
|---|---|---|---|---|---|---|
| Ex | | Human | Human | Human | Human | Human |
| No | Structural Name | IC50 | IC50 | IC50 | IC50 | IC50 |
| 1A | 4-amino-2-methoxy-6-[4- | — | — | — | 0.0904 μM | |
| | (trifluoromethoxy)phenyl]- | | | | (n = 4) | |
| | 7,8-dihydropyrido[4,3- | | | | | |
| | d]pyrimidin-5(6H)-one | | | | | |
| 1B | 4-amino-6-(4-ethylphenyl)- | — | — | — | 0.0527 μM | |
| | 2-methoxy-7,8- | | | | (n = 2) | |
| | dihydropyrido[4,3- | | | | | |
| | d]pyrimidin-5(6H)-one | | | | | |
| 1C | 4-amino-6-[4- | — | — | — | 0.0110 μM | |
| | (cyclopropylmethyl)- | | | | (n = 3) | |
| | phenyl]-2-methoxy-7,8- | | | | | |
| | dihydropyrido[4,3- | | | | | |
| | d]pyrimidin-5(6H)-one | | | | | |
| 1D | 4-amino-6-(2,3-dihydro- | — | — | — | 0.0404 μM | |
| | 1H-inden-5-yl)-2-methoxy- | | | | (n = 4) | |
| | 7,8-dihydropyrido-[4,3- | | | | | |
| | d]pyrimidin-5(6H)-one | | | | | |
| 1E | 4-amino-6-(4- | — | — | — | 0.0288 μM | |
| | cyclopropylphenyl)-2- | | | | (n = 4) | |
| | methoxy-7,8- | | | | | |
| | dihydropyrido[4,3- | | | | | |
| | d]pyrimidin-5(6H)-one | | | | | |
| 1F | 4-amino-6-[4-(2,2- | | | | | 0.192 uM |
| | dimethylpropanoyl)phenyl]- | | | | | (n = 2) |
| | 2-methoxy-7,8- | | | | | |
| | dihydropyrido[4,3- | | | | | |
| | d]pyrimidin-5(6H)-one | | | | | |
| 1G | 4-amino-6-(3-fluoro-4- | — | — | — | 0.00966 μM | |
| | isopropyl-phenyl)-2- | | | | (n = 3) | |
| | methoxy-7,8- | | | | | |
| | dihydropyrido[4,3- | | | | | |
| | d]pyrimidin-5(6H)-one | | | | | |
| 1H | 4-amino-6-[4-(3,3- | — | — | — | 0.0576 μM | |
| | difluorocyclobutyl)phenyl]- | | | | (n = 3) | |
| | 2-methoxy-7,8- | | | | | |
| | dihydropyrido[4,3- | | | | | |
| | d]pyrimidin-5(6H)-one | | | | | |
| 1I | 4-amino-6-[4-(trans-3- | — | — | — | 0.0958 μM | |
| | hydroxycyclobutyl)phenyl]- | | | | (n = 3) | |
| | 2-methoxy-7,8- | | | | | |
| | dihydropyrido[4,3- | | | | | |
| | d]pyrimidin-5(6H)-one | | | | | |
| 1J | 4-amino-6-[4-(cis-3- | — | — | — | 0.0598 | |
| | hydroxycyclobutyl)phenyl]- | | | | (n = 2) | |
| | 2-methoxy-7,8- | | | | | |
| | dihydropyrido[4,3- | | | | | |
| | d]pyrimidin-5(6H)-one | | | | | |
| 1K | 4-amino-6-[3-(2-hydroxy- | | | | | 0.831 uM |
| | 1,1-dimethylethyl) phenyl]- | | | | | (n = 4) |
| | 2-methoxy-7,8- | | | | | |
| | dihydropyrido[4,3- | | | | | |
| | d]pyrimidin-5(6H)-one | | | | | |
| 1L | 4-amino-2-methoxy-6-{4- | | | | | 0.0348 uM |
| | [1-methyl-1-(1,3-oxazol-2- | | | | | (n = 8) |
| | yl)ethyl]phenyl}-7,8- | | | | | |
| | dihydropyrido[4,3- | | | | | |
| | d]pyrimidin-5(6H)-one | | | | | |
| 1M | 4-amino-2-methoxy-6-{4- | | | | | 0.0113 uM |
| | [1-methyl-1-(1,3-oxazol-5- | | | | | (n = 6) |
| | yl)ethyl]phenyl}-7,8- | | | | | |
| | dihydropyrido[4,3- | | | | | |
| | d]pyrimidin-5(6H)-one | | | | | |
| 1N | 4-amino-2-methoxy-6-{4- | | | | | 0.0319 uM |
| | [1-methyl-1-(1H-pyrazol-3- | | | | | (n = 5) |
| | yl)ethyl]phenyl}-7,8- | | | | | |
| | dihydropyrido[4,3- | | | | | |
| | d]pyrimidin-5(6H)-one | | | | | |
| 1O | 4-amino-6-{4-[1- | — | — | — | 0.0359 μM | |
| | (hydroxymethyl)cyclobutyl] | | | | (n = 5) | |
| | phenyl}-2-methoxy-7,8- | | | | | |
| | dihydropyrido[4,3- | | | | | |
| | d]pyrimidin-5(6H)-one | | | | | |
| 1P | 1-[4-(4-amino-2-methoxy- | — | — | — | 0.0671 μM | |
| | 5-oxo-7,8- | | | | (n = 3) | |
| | dihydropyrido[4,3- | | | | | |
| | d]pyrimidin-6(5H)- | | | | | |
| | yl)phenyl]- | | | | | |
| | cyclobutanecarboxylic | | | | | |
| | acid, Potassium salt | | | | | |
| 1S | 4-amino-6-[4-(2-hydroxy-2- | | | | | 0.0443 uM |
| | methylpropyl)phenyl]-2- | | | | | (n = 6) |
| | methoxy-7,8- | | | | | |
| | dihydropyrido[4,3- | | | | | |
| | d]pyrimidin-5(6H)-one | | | | | |
| 1T | 4-amino-6-[4-(2-hydroxy- | — | — | — | 0.0529 μM | |
| | 1,1-dimethylethyl) phenyl]- | | | | (n = 11) | |
| | 2-methoxy-7,8- | | | | | |
| | dihydropyrido[4,3- | | | | | |
| | d]pyrimidin-5(6H)-one | | | | | |
| 1U | 4-amino-6-(1,1-dimethyl- | | | | | 0.229 uM |
| | 1,3-dihydro-2-benzofuran- | | | | | (n = 5) |
| | 5-yl)-2-methoxy-7,8- | | | | | |
| | dihydropyrido[4,3- | | | | | |
| | d]pyrimidin-5(6H)-one | | | | | |
| 1V | 4-amino-6-[4-(1- | | | | | 0.113 uM |
| | hydroxycyclohexyl)phenyl]- | | | | | (n = 6) |
| | 2-methoxy-7,8- | | | | | |
| | dihydropyrido[4,3- | | | | | |
| | d]pyrimidin-5(6H)-one | | | | | |
| 1W | 4-amino-6-[4-(1-ethyl-1- | | | | | 0.0340 uM |
| | hydroxypropyl)phenyl]-2- | | | | | (n = 8) |
| | methoxy-7,8- | | | | | |
| | dihydropyrido[4,3- | | | | | |
| | d]pyrimidin-5(6H)-one | | | | | |
| 1X | 4-amino-2-methoxy-6-{4- | — | — | — | 0.00519 μM | |
| | [2,2,2-trifluoro-1-hydroxy- | | | | (n = 4) | |
| | 1-(trifluoromethyl) | | | | | |
| | ethyl]phenyl}-7,8- | | | | | |
| | dihydropyrido[4,3- | | | | | |
| | d]pyrimidin-5(6H)-one | | | | | |
| 1Y | 4-amino-2-methoxy-6-[4- | — | — | — | 0.0460 μM | |
| | (2,2,2-trifluoro-1- | | | | (n = 4) | |
| | hydroxyethyl)phenyl]-7,8- | | | | | |
| | dihydropyrido[4,3- | | | | | |
| | d]pyrimidin-5(6H)-one | | | | | |
| 1Z | 4-amino-6-(4- | — | — | — | 0.00492 μM | |
| | isobutylphenyl)-2- | | | | (n = 2) | |
| | methoxy-7,8- | | | | | |
| | dihydropyrido[4,3- | | | | | |
| | d]pyrimidin-5(6H)-one | | | | | |
| 2A | methyl 4-(4-amino-2- | — | — | — | 0.0503 μM | |
| | methoxy-5-oxo-7,8- | | | | (n = 2) | |
| | dihydropyrido[4,3- | | | | | |
| | d]pyrimidin-6(5H)- | | | | | |
| | yl)benzoate | | | | | |
| 2B | 4-amino-6-[4-(1-hydroxy-1- | — | — | — | 0.177 μM | |
| | methylethyl)phenyl]-2- | | | | (n = 3) | |
| | methoxy-7,8- | | | | | |
| | dihydropyrido[4,3- | | | | | |
| | d]pyrimidin-5(6H)-one | | | | | |
| 2C | 4-amino-6-[4-(1-fluoro-1- | — | — | — | 0.0866 μM | |
| | methyl-ethyl)phenyl]-2- | | | | (n = 6) | |
| | methoxy-7,8- | | | | | |
| | dihydropyrido[4,3- | | | | | |
| | d]pyrimidin-5(6H)-one | | | | | |
| 2D | 4-amino-2-methoxy-6-[4- | — | — | — | 0.0346 μM | |
| | (1-methoxy-1- | | | | (n = 3) | |
| | methylethyl)phenyl]-7,8- | | | | | |
| | dihydropyrido[4,3- | | | | | |
| | d]pyrimidin-5(6H)-one | | | | | |
| 2E | 4-(4-amino-2-methoxy-5- | — | — | — | 1.48 μM | |
| | oxo-7,8-dihydropyrido[4,3- | | | | (n = 3) | |
| | d]pyrimidin-6(5H)- | | | | | |
| | yl)benzoic acid | | | | | |
| 2F | 2,2-dimethylpropyl 4-(4- | | | | 0.112 μM | 0.0551 uM |
| | amino-2-methoxy-5-oxo- | | | | (n = 2) | (n = 6) |
| | 7,8-dihydropyrido[4,3- | | | | | |
| | d]pyrimidin-6(5H)- | | | | | |
| | yl)benzoate | | | | | |
| 2G | cyclohexyl 4-(4-amino-2- | | | | 0.0744 μM | |
| | methoxy-5-oxo-7,8- | | | | (n = 2) | |
| | dihydropyrido[4,3- | | | | | |
| | d]pyrimidin-6(5H)- | | | | | |
| | yl)benzoate | | | | | |
| 2H | 4-(4-amino-2-methoxy-5- | — | — | — | 1.37 μM | |
| | oxo-7,8-dihydropyrido [4,3- | | | | (n = 3) | |
| | d]pyrimidin-6(5H)-yl)-N,N- | | | | | |
| | dimethylbenzamide | | | | | |
| 3A | methyl 2-[4-(4-amino-2- | — | — | — | 0.0137 μM | |
| | methoxy-5-oxo-7,8- | | | | (n = 4) | |
| | dihydropyrido[4,3- | | | | | |
| | d]pyrimidin-6(5H)- | | | | | |
| | yl)phenyl]-2- | | | | | |
| | methylpropanoate | | | | | |
| 3B | 4-amino-6-[4-(1-isoxazol- | | | | | 0.0550 uM |
| | 3-yl-1-methylethyl) | | | | | (n = 8) |
| | phenyl]-2-methoxy-7,8- | | | | | |
| | dihydropyrido[4,3- | | | | | |
| | d]pyrimidin-5(6H)-one | | | | | |
| 3C | 4-amino-6-[4-(1-isoxazol- | | | | | 0.00450 uM |
| | 5-yl-1-methylethyl) | | | | | (n = 8) |
| | phenyl]-2-methoxy-7,8- | | | | | |
| | dihydropyrido[4,3- | | | | | |
| | d]pyrimidin-5(6H)-one | | | | | |
| 3D | 2-[4-(4-amino-2-methoxy- | — | — | — | 0.0462 μM | |
| | 5-oxo-7,8- | | | | (n = 8) | |
| | dihydropyrido[4,3- | | | | | |
| | d]pyrimidin-6(5H)- | | | | | |
| | yl)phenyl]-2-methyl- | | | | | |
| | propanoic acid | | | | | |
| 3F | 4-amino-2-methoxy-6-{4- | — | — | — | 0.0515 μM | |
| | [1-methyl-1-(3-methyl- | | | | (n = 6) | |
| | 1,2,4-oxadiazol-5- | | | | | |
| | yl)ethyl]phenyl}-7,8- | | | | | |
| | dihydropyrido[4,3- | | | | | |
| | d]pyrimidin-5(6H)-one | | | | | |
| 3G | 2-[4-(4-amino-2-methoxy- | — | — | — | 0.126 μM | |
| | 5-oxo-7,8- | | | | (n = 2) | |
| | dihydropyrido[4,3- | | | | | |
| | d]pyrimidin-6(5H)- | | | | | |
| | yl)phenyl]-N,2-dimethyl- | | | | | |
| | propanamide | | | | | |
| 3H | 2-[4-(4-amino-2-methoxy- | — | — | — | 0.260 μM | |
| | 5-oxo-7,8- | | | | (n = 2) | |
| | dihydropyrido[4,3- | | | | | |
| | d]pyrimidin-6(5H)- | | | | | |
| | yl)phenyl]-N,N,2- | | | | | |
| | trimethylpropanamide | | | | | |
| 3I | 2-[4-(4-amino-2-methoxy- | — | — | — | 0.0528 μM | |
| | 5-oxo-7,8- | | | | (n = 3) | |
| | dihydropyrido[4,3- | | | | | |
| | d]pyrimidin-6(5H)- | | | | | |
| | yl)phenyl]-2-methyl- | | | | | |
| | propanamide | | | | | |
| 3K | 4-amino-2-methoxy-6-{4- | | | | 0.795 uM | |
| | [1-methyl-1-(1-methyl-1H- | | | | (n = 1) | |
| | 1,2,4-triazol-5- | | | | | |
| | yl)ethyl]phenyl}-7,8- | | | | | |
| | dihydropyrido[4,3- | | | | | |
| | d]pyrimidin-5(6H)-one | | | | | |
| 3L | 4-amino-2-methoxy-6-{4- | | | | 0.0689 uM | 0.0673 uM |
| | [1-methyl-1-(1,2,4- | | | | (n = 2) | |
| | oxadiazol-5- | | | | | |
| | yl)ethyl]phenyl}-7,8- | | | | | |
| | dihydropyrido[4,3- | | | | | |
| | d]pyrimidin-5(6H)-one | | | | | |
| 3M | 4-amino-2-methoxy-6-{4- | | | | 0.0778 uM | 0.0552 uM |
| | [1-methyl-1-(1,2,4- | | | | (n = 1) | (n = 2) |
| | oxadiazol-3- | | | | | |
| | yl)ethyl]phenyl}-7,8- | | | | | |
| | dihydropyrido[4,3- | | | | | |
| | d]pyrimidin-5(6H)-one | | | | | |
| 3N | 4-amino-2-methoxy-6-{4- | | | | 0.0785 uM | |
| | [1-methyl-1-(1H-1,2,4- | | | | (n = 2) | |
| | triazol-5-yl)ethyl]phenyl}- | | | | | |
| | 7,8-dihydropyrido[4,3- | | | | | |
| | d]pyrimidin-5(6H)-one | | | | | |
| 3O | 2-[4-(4-amino-2-methoxy- | — | — | — | 0.0883 μM | |
| | 5-oxo-7,8- | | | | (n = 5) | |
| | dihydropyrido[4,3- | | | | | |
| | d]pyrimidin-6(5H)- | | | | | |
| | yl)phenyl]-2- | | | | | |
| | methylpropanenitrile | | | | | |
| 3P | 4-amino-2-methoxy-6-{4- | — | — | — | 0.0209 μM | |
| | [1-methyl-1-(1H-tetrazol-5- | | | | (n = 3) | |
| | yl)ethyl]phenyl}-7,8- | | | | | |
| | dihydropyrido[4,3- | | | | | |
| | d]pyrimidin-5(6H)-one | | | | | |
| 3Q | 4-amino-2-methoxy-6-{4- | | | | | 0.0953 uM |
| | [1-methyl-1-(1-methyl-1H- | | | | | (n = 6) |
| | tetrazol-5-yl)ethyl]phenyl}- | | | | | |
| | 7,8-dihydropyrido[4,3- | | | | | |
| | d]pyrimidin-5(6H)-one | | | | | |
| 3R | 4-amino-2-methoxy-6-{4- | | | | | 0.0211 uM |
| | [1-methyl-1-(2-methyl-2H- | | | | | (n = 6) |
| | tetrazol-5-yl)ethyl]phenyl}- | | | | | |
| | 7,8-dihydropyrido[4,3- | | | | | |
| | d]pyrimidin-5(6H)-one | | | | | |
| 3T | tert-butyl {2-[4-(4-amino-2- | | | | | 0.0196 uM |
| | methoxy-5-oxo-7,8- | | | | | (n = 4) |
| | dihydropyrido[4,3- | | | | | |
| | d]pyrimidin-6(5H)- | | | | | |
| | yl)phenyl]-2- | | | | | |
| | methylpropyl}carbamate | | | | | |
| 3U | N-{2-[4-(4-amino-2- | — | — | — | 0.0843 μM | |
| | methoxy-5-oxo-7,8- | | | | (n = 4) | |
| | dihydropyrido[4,3- | | | | | |
| | d]pyrimidin-6(5H)- | | | | | |
| | yl)phenyl]-2- | | | | | |
| | methylpropyl}acetamide | | | | | |
| 4A | 1-[4-(4-amino-2-methoxy- | | | | | 0.0207 uM |
| | 5-oxo-7,8- | | | | | (n = 9) |
| | dihydropyrido[4,3- | | | | | |
| | d]pyrimidin-6(5H)- | | | | | |
| | yl)phenyl] | | | | | |
| | cyclobutanecarbonitrile | | | | | |
| 5A | 1-[4-(4-amino-2-methoxy- | | | | | 0.0786 uM |
| | 5-oxo-7,8- | | | | | (n = 14) |
| | dihydropyrido[4,3- | | | | | |
| | d]pyrimidin-6(5H)- | | | | | |
| | yl)phenyl] | | | | | |
| | cyclobutanecarboxamide | | | | | |
| 5B | 1-[4-(4-amino-2-methoxy- | | | | | 0.184 uM |
| | 5-oxo-7,8- | | | | | (n = 8) |
| | dihydropyrido[4,3- | | | | | |
| | d]pyrimidin-6(5H)-yl) | | | | | |
| | phenyl] | | | | | |
| | cyclohexanecarboxamide | | | | | |
| 5C | 1-[4-(4-amino-2-methoxy- | | | | | 0.217 uM |
| | 5-oxo-7,8- | | | | | (n = 10) |
| | dihydropyrido[4,3- | | | | | |
| | d]pyrimidin-6(5H)- | | | | | |
| | yl)phenyl] | | | | | |
| | cyclopentanecarboxamide | | | | | |
| 6A | 4-amino-6-(4-tert- | — | — | 0.0490 μM | 0.00888 μM | |
| | butylphenyl)-2-methoxy- | | | (n = 4) | (n = 11) | |
| | 7,8-dihydropyrido[4,3- | | | | | |
| | d]pyrimidin-5(6H)-one | | | | | |
| 6B | 4-amino-6-(3- | — | — | — | 0.873 μM | |
| | isopropylphenyl)-2- | | | | (n = 3) | |
| | methoxy-7,8- | | | | | |
| | dihydropyrido[4,3- | | | | | |
| | d]pyrimidin-5(6H)-one | | | | | |
| 6C | 4-amino-6-(3-tert- | — | — | — | 0.0377 μM | |
| | butylphenyl)-2-methoxy- | | | | (n = 3) | |
| | 7,8-dihydropyrido[4,3- | | | | | |
| | d]pyrimidin-5(6H)-one | | | | | |
| 6D | 4-amino-6-(4-tert- | — | — | — | 0.899 μM | |
| | butylphenyl)-2-ethoxy-7,8- | | | | (n = 1) | |
| | dihydropyrido[4,3- | | | | | |
| | d]pyrimidin-5(6H)-one | | | | | |
| 6E | 4-amino-6-(4- | — | — | 0.0585 μM | 0.0171 μM | |
| | isopropylphenyl)-2- | | | (n = 4) | (n = 2) | |
| | methoxy-7,8- | | | | | |
| | dihydropyrido[4,3- | | | | | |
| | d]pyrimidin-5(6H)-one | | | | | |
| 6F | 4-amino-2-methoxy-6-[4- | — | — | — | 0.329 μM | |
| | (trifluoromethyl)phenyl]- | | | | (n = 5) | |
| | 7,8-dihydropyrido[4,3- | | | | | |
| | d]pyrimidin-5(6H)-one | | | | | |
| 6G | 4-amino-6-(2-fluoro-4- | — | — | — | 0.0198 μM | |
| | isopropyl-phenyl)-2- | | | | (n = 2) | |
| | methoxy-7,8- | | | | | |
| | dihydropyrido[4,3- | | | | | |
| | d]pyrimidin-5(6H)-one | | | | | |
| 6H | 4-amino-6-(4-tert-butyl-3- | — | — | — | 0.0126 μM | |
| | fluorophenyl)-2-methoxy- | | | | (n = 1) | |
| | 7,8-dihydropyrido[4,3- | | | | | |
| | d]pyrimidin-5(6H)-one | | | | | |
| 6I | 4-amino-6-(4-tert-butyl-2- | — | — | — | 0.00490 μM | |
| | fluorophenyl)-2-methoxy- | | | | (n = 2) | |
| | 7,8-dihydropyrido[4,3- | | | | | |
| | d]pyrimidin-5(6H)-one | | | | | |
| 6J | 4-amino-2-methoxy-6- | | | | 0.256 uM | |
| | (1,3,3-trimethyl-2-oxo-2,3- | | | | (n = 3) | |
| | dihydro-1H-indol-6-yl)-7,8- | | | | | |
| | dihydropyrido[4,3- | | | | | |
| | d]pyrimidin-5(6H)-one | | | | | |
| 6K | 4-amino-2-methoxy-6-[4- | — | — | — | 0.0327 μM | |
| | (2,2,2-trifluoro-1,1- | | | | (n = 5) | |
| | dimethylethyl)phenyl]-7,8- | | | | | |
| | dihydropyrido[4,3- | | | | | |
| | d]pyrimidin-5(6H)-one | | | | | |
| 7C | {trans-4-[4-(4-amino-2- | — | — | 0.133 μM | 0.0325 μM | |
| | methoxy-5-oxo-7,8- | | | (n = 4) | (n = 2) | |
| | dihydropyrido[4,3- | | | | | |
| | d]pyrimidin-6(5H)- | | | | | |
| | yl)phenyl]- | | | | | |
| | cyclohexyl}acetonitrile | | | | | |
| 7E | 4-amino-6-(3-fluoro-4- | — | — | — | 0.0387 μM | |
| | {trans-4-[(3-methyl-1,2,4- | | | | (n = 3) | |
| | oxadiazol-5- | | | | | |
| | yl)methyl]cyclohexyl}phenyl)- | | | | | |
| | 2-methoxy-7,8- | | | | | |
| | dihydropyrido[4,3- | | | | | |
| | d]pyrimidin-5(6H)-one | | | | | |
| 8A | N-{1-[4-(4-amino-2- | — | — | — | 1.57 μM | |
| | methoxy-5-oxo-7,8- | | | | (n = 1) | |
| | dihydropyrido[4,3- | | | | | |
| | d]pyrimidin-6(5H)- | | | | | |
| | yl)phenyl]-1- | | | | | |
| | methylethyl}acetamide, | | | | | |
| | Hydrochloride salt | | | | | |
| 9A | 4-amino-6-(3,4- | — | — | — | 0.0689 μM | |
| | dichlorophenyl)-2- | | | | (n = 5) | |
| | methoxy-7,8- | | | | | |
| | dihydropyrido[4,3- | | | | | |
| | d]pyrimidin-5(6H)-one | | | | | |
| 9B | 4-amino-6-(2,3-dihydro- | — | — | — | 0.104 μM | |
| | 1,4-benzodioxin-6-yl)-2- | | | | (n = 4) | |
| | methoxy-7,8- | | | | | |
| | dihydropyrido[4,3- | | | | | |
| | d]pyrimidin-5(6H)-one | | | | | |
| 9C | 4-amino-2-methoxy-6- | | | | 0.594 uM | |
| | phenyl-7,8- | | | | (n = 3) | |
| | dihydropyrido[4,3- | | | | | |
| | d]pyrimidin-5(6H)-one | | | | | |
| 9D | 4-amino-6-(4-chloro-3- | — | — | — | 0.101 μM | |
| | fluorophenyl)-2-methoxy- | | | | (n = 5) | |
| | 7,8-dihydropyrido[4,3- | | | | | |
| | d]pyrimidin-5(6H)-one | | | | | |
| 9E | 4-amino-6-(4-iodophenyl)- | — | — | 0.124 μM | — | |
| | 2-methoxy-7,8- | | | (n = 1) | | |
| | dihydropyrido[4,3- | | | | | |
| | d]pyrimidin-5(6H)-one | | | | | |
| 9F | 4-amino-6-(4- | — | — | — | 0.747 μM | |
| | chlorophenyl)-2-methoxy- | | | | (n = 5) | |
| | 7,8-dihydropyrido[4,3- | | | | | |
| | d]pyrimidin-5(6H)-one | | | | | |
| 9G | 4-amino-6-(4- | — | — | — | 0.102 μM | |
| | isopropoxyphenyl)-2- | | | | (n = 6) | |
| | methoxy-7,8- | | | | | |
| | dihydropyrido[4,3- | | | | | |
| | d]pyrimidin-5(6H)-one | | | | | |
| 9H | 4-amino-6-[4- | — | — | — | 0.115 μM | |
| | (cyclopropylmethoxy)phenyl]- | | | | (n = 2) | |
| | 2-methoxy-7,8- | | | | | |
| | dihydropyrido[4,3- | | | | | |
| | d]pyrimidin-5(6H)-one | | | | | |
| 9I | 4-amino-6-(4- | — | — | — | 0.0841 μM | |
| | isobutoxyphenyl)-2- | | | | (n = 4) | |
| | methoxy-7,8- | | | | | |
| | dihydropyrido[4,3- | | | | | |
| | d]pyrimidin-5(6H)-one | | | | | |
| 9J | 4-amino-6-(1H- | — | — | — | 1.40 μM | |
| | benzimidazol-6-yl)-2- | | | | (n = 2) | |
| | methoxy-7,8- | | | | | |
| | dihydropyrido-[4,3- | | | | | |
| | d]pyrimidin-5(6H)-one, | | | | | |
| | Hydrochloride salt | | | | | |
| 9K | 4-amino-2-methoxy-6-[4- | | | | 0.0632 uM | |
| | (2-methyltetrahydrofuran- | | | | (n = 3) | |
| | 2-yl)phenyl]-7,8- | | | | | |
| | dihydropyrido[4,3- | | | | | |
| | d]pyrimidin-5(6H)-one | | | | | |
| 9L | 4-amino-2-methoxy-6-[4- | — | — | — | 0.0774 μM | |
| | (3,3,3- | | | | (n = 2) | |
| | trifluoropropyl)phenyl]-7,8- | | | | | |
| | dihydropyrido[4,3- | | | | | |
| | d]pyrimidin-5(6H)-one | | | | | |
| 9M | 4-amino-6-(2,2-dimethyl- | | | | | 1.38 uM |
| | 2,3-dihydro-1-benzofuran- | | | | | (n = 4) |
| | 5-yl)-2-methoxy-7,8- | | | | | |
| | dihydropyrido[4,3- | | | | | |
| | d]pyrimidin-5(6H)-one | | | | | |
| 9N | 4-amino-6-[4-(tert- | — | — | — | 0.0239 μM | |
| | butylthio)-phenyl]-2- | | | | (n = 3) | |
| | methoxy-7,8- | | | | | |
| | dihydropyrido[4,3- | | | | | |
| | d]pyrimidin-5(6H)-one | | | | | |
| 9O | 4-amino-6-[4-(tert- | — | — | — | 0.648 μM | |
| | butylsulfinyl)-phenyl]-2- | | | | (n = 3) | |
| | methoxy-7,8- | | | | | |
| | dihydropyrido[4,3- | | | | | |
| | d]pyrimidin-5(6H)-one | | | | | |
| 9P | 4-amino-6-[4-(tert- | — | — | — | 0.784 μM | |
| | butylsulfonyl)-phenyl]-2- | | | | (n = 3) | |
| | methoxy-7,8- | | | | | |
| | dihydropyrido[4,3- | | | | | |
| | d]pyrimidin-5(6H)-one | | | | | |
| 9Q | 4-amino-6-(4-iodophenyl)- | 5.03 μM | — | 13.7 μM | — | |
| | 2-methyl-7,8- | (n = 1) | | (n = 1) | | |
| | dihydropyrido[4,3- | | | | | |
| | d]pyrimidin-5(6H)-one | | | | | |
| 9R | 4-amino-6-{4-[4-(2- | 1.03 μM | — | — | — | |
| | hydroxyethyl)- | (n = 1) | | | | |
| | cyclohexyl]phenyl}-2- | | | | | |
| | methyl-7,8- | | | | | |
| | dihydropyrido[4,3- | | | | | |
| | d]pyrimidin-5(6H)-one | | | | | |
| 9T | {4-[4-(4-amino-2-methyl-5- | 1.89 μM | — | — | — | |
| | oxo-7,8-dihydropyrido[4,3- | (n = 1) | | | | |
| | d]pyrimidin-6(5H)- | | | | | |
| | yl)phenyl]- | | | | | |
| | cyclohexyl}acetonitrile | | | | | |
| 10A | 4-amino-6-[4-(1-hydroxy- | — | — | — | 0.0588 μM | |
| | 2,2-dimethylpropyl)phenyl]- | | | | (n = 4) | |
| | 2-methoxy-7,8- | | | | | |
| | dihydropyrido[4,3- | | | | | |
| | d]pyrimidin-5(6H)-one | | | | | |
| 10D | 4-amino-2-methoxy-6-[4- | | | | 0.0279 uM | |
| | (1-methoxy-2,2- | | | | (n = 3) | |
| | dimethylpropyl)phenyl]- | | | | | |
| | 7,8-dihydropyrido[4,3- | | | | | |
| | d]pyrimidin-5(6H)-one | | | | | |
| 11A | 4-amino-6-(4-tert- | — | — | — | 0.117 μM | |
| | butylphenyl)-7,8- | | | | (n = 3) | |
| | dihydropyrido[4,3- | | | | | |
| | d]pyrimidin-5(6H)-one | | | | | |

### In Vivo Assay for Glucose Lowering

Oral glucose tolerance tests (“OGTT”) have been in use in humans since, at least, the 1930s, Pincus et al., Am J Med Sci 188, 782 (1934), and are routinely used in the diagnosis of human diabetes, though not to evaluate the efficacy of therapeutic agents in patients.

KK mice have been used to evaluate glitazones (Fujita, et al., Diabetes, 32, 804-810 (1983); Fujiwara, et al., Diabetes, 37, 1549-48 (1988); Izumi et al. Biopharm Durg Dispos, 18, 247-257 (1997), metformin (Reddi, et al., Diabet Metabl, 19, 44-51 (1993), glucosidase inhibitors (Hamada, et al., Jap Pharmacol Ther, 17, 17-28 (1988); Matsuo, et al., Am J Clin Nutr, 55, 314S-317S (1992)), and the extra-pancreatic effects of sulfonylureas (Kameda, et a., Arzenim Forsch./Drug Res, 32, 39044 (1982); and Muller, et al., Horm Metabl Res, 28, 469-487 (1990)).

KK mice are derived from an inbred line first established by Kondo et al. (Kondo, et al., Bull Exp Anim, 6, 107-112 (1957)). The mice spontaneously develop a hereditary form of polygenic diabetes that progresses to cause renal, retinal and neurological complications analogous to those seen in human diabetic subjects, but they do not require insulin or other medication for survival. Another aspect of the invention is directed to the use of KK mice to evaluate the effects of insulin secretagogue agents in the context of an oral glucose tolerance test.

### In Vivo Assay for Food Intake

The following screen may be used to evaluate the efficacy of test compounds for inhibiting food intake in Sprague-Dawley rats after an overnight fast.

Male Sprague-Dawley rats are individually housed and fed powdered chow. They are maintained on a 12 hour light/dark cycle and received food and water ad libitum. The animals are acclimated to the vivarium for a period of one week before testing is conducted. Testing is completed during the light portion of the cycle.

To conduct the food intake efficacy screen, rats are transferred to individual test cages without food the afternoon prior to testing, and the rats are fasted overnight. After the overnight fast, rats are dosed the following morning with vehicle or test compounds. A known antagonist is dosed (3 mg/kg) as a positive control, and a control group receives vehicle alone (no compound). The test compounds are dosed at ranges between 0.1 and 100 mg/kg depending upon the compound. The standard vehicle is 0.5% (w/v) methylcellulose in water and the standard route of administration is oral. However, different vehicles and routes of administration may be used to accommodate various compounds when required. Food is provided to the rats 30 minutes after dosing and an Oxymax automated food intake system (Columbus Instruments, Columbus, Ohio) is started. Individual rat food intake is recorded continuously at 10-minute intervals for a period of two hours. When required, food intake is recorded manually using an electronic scale; food is weighed every 30 minutes after food is provided up to four hours after food is provided. Compound efficacy is determined by comparing the food intake pattern of compound-treated rats to vehicle and the standard positive control.

Throughout this application, various publications are referenced. The disclosures of these publications in their entireties are hereby incorporated by reference into this application for all purposes.

It will be apparent to those skilled in the art that various modifications and variations can be made in the invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims and the application as a whole.

What is claimed is:

1. A compound of Formula (I) 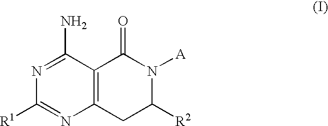
wherein
R¹ is hydrogen, (C₁-C₂)alkyl, or (C₁-C₂)alkoxy;
R² is hydrogen or (C₁-C₂)alkyl;
A is a group of formulae (1A), (1B), (1C) or (1D), 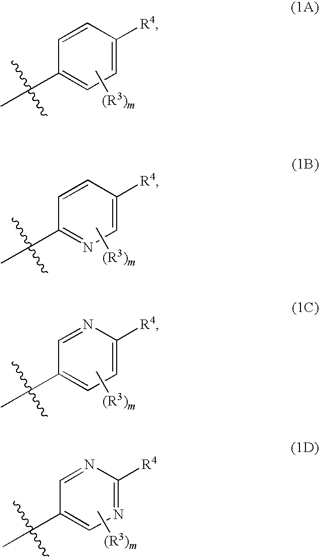
where each R³ is independently halogen, OH, (C₁-C₄)alkyl, cyano, (C₃-C₆)cycloalkyl or (C₁-C₄)alkoxy; and m is 0, 1, 2 or 3;
R⁴ is hydrogen, halogen, or a chemical moiety selected from the group consisting of:
(ii) taken together with R³ to form a 5- to 6-membered carbocyclic fused ring, a 5- to 6-membered heterocyclic fused ring containing 1 to 2 heteroatoms each independently selected from O, N or S, or a 5- to 6-membered heteroaryl fused ring containing 1 to 2 heteroatoms each independently selected form O, N or S wherein the carbocyclic, heterocyclic and heteroaryl fused rings are optionally substituted with one to four substituents selected from the group consisting of (C₁-C₄)alkyl, (C₁-C₄)alkoxy, (C₁-C₄)haloalkyl, (C₁-C₄)haloalkoxy, -hydroxyl, halogen, cyano, oxo, —NH₂, —NH((C₁-C₄)alkyl), —N((C₁-C₄)alkyl)₂, —C(O)—OH, —C(O)—(C₁-C₄)alkoxy, —C(O)—NH₂, —C(O)—NH((C₁-C₄)alkyl), and —C(O)—N((C₁-C₄)alkyl)₂;
(ii) (C₁-C₆)alkyl optionally substituted with one or more substitutents selected from the group consisting of hydroxy, cyano, (C₁-C₆)alkoxy, halo-substituted (C₁-C₆)alkoxy, halogen, —NH₂, NH, oxo —S(C₁-C₄)alkyl, —SO(C₁-C₄)alkyl, —SO₂(C₁-C₄)alkyl, —O—SO₂(C₁-C₄)alkyl, —(CH₂)ₚC(O)—N(R^{6a})(R^{6b}), —(CH₂)ₚNH—C(O)(C₁-C₄)alkyl), —(CH₂)ₚNH—C(O)(C₁-C₄)alkoxy, —(CH₂)ₚC(O)—O(R^{6c}), —(CH₂)ₚOH, a 3- to 6-membered carbocyclic group, an aryl group, and a 5- to 6-membered heteroaryl group containing 1 to 4 heteroatoms each independently selected from O, S, and N, wherein the carbocyclic, aryl and heteroaryl groups are optionally substituted with (C₁-C₄)alkyl, —(CH₂)ₚC(O)—N(R^{6a})(R^{6b}), —(CH₂)ₚNH—C(O)(C₁-C₄)alkyl), —(CH₂)ₚC(O)—O(R^{6c}), —(CH₂)ₚNH—C(O)(C₁-C₄)alkoxy, or —(CH₂)ₚOH;
(iii) (C₁-C₆)alkoxy optionally substituted with one or more substitutents selected from the group consisting of hydroxy, cyano, (C₁-C₄)alkyl, halo-substituted (C₁-C₄)alkyl, halo-substituted (C₁-C₄)alkoxy, halogen, —NH₂, oxo or—a 3- to 6-membered cycloalkyl group;
(iv) —S(C₁-C₄)alkyl, —SO(C₁-C₄)alkyl, or —SO₂(C₁-C₄)alkyl, or
(v) (CH₂)ₒ—C(O)—OR⁵, (CH₂)ₒ—C(O)—(C₁-C₄)alkoxy-R⁷, (CH₂)ₒ—C(O)—N(R⁵)(R⁶), or (CH₂)ₒ—C(O)—R⁵,
(vii) 3- to 6-membered carbocyclic ring or a 3- to 6-membered heterocyclic ring containing 1 to 2 heteroatoms each independently selected from O, N or S, wherein the carbocyclic and heterocyclic rings are optionally substituted with one to four substituents selected from the group consisting of —(CH₂)ₙC(O)—O(R⁵), —(CH₂)ₙOH, (C₁-C₄)alkoxy, —(CH₂)ₙC(O)—N(R⁵)(R⁶), —(CH₂)ₙOH, (C₁-C₄)alkyl, (C₁-C₄)haloalkyl, (C₁-C₄)haloalkoxy, hydroxyl, halogen, cyano, oxo, a 5- to 6-membered heteroaryl containing 1 to 3 heteroatoms each independently selected from O, N and S, and —N(R⁵)(R⁶), wherein said heteroaryl is optionally substituted with 1 to 3 substituents each independently selected from OH, halogen, or (C₁-C₄)alkyl;
n is 0 or 1;
o is 0, 1, or 2;
p is 0, 1, or 2;
R⁵, R⁶, R^{6a}, R^{6b}, and R^{6c} are each independently H or (C₁-C₄)alkyl; and
R⁷ is H, (C₁-C₄)alkyl, (C₃-C₆)cycloalkyl, or aryl;
or a pharmaceutically acceptable salt thereof.

2. A compound of Formula (I) 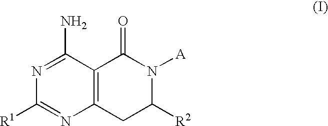
wherein
R¹ is hydrogen, (C₁-C₂)alkyl, or (C₁-C₂)alkoxy;
R² is hydrogen or (C₁-C₂)alkyl;
A is a group of formulae (1A), (1B), (1C) or (1D), 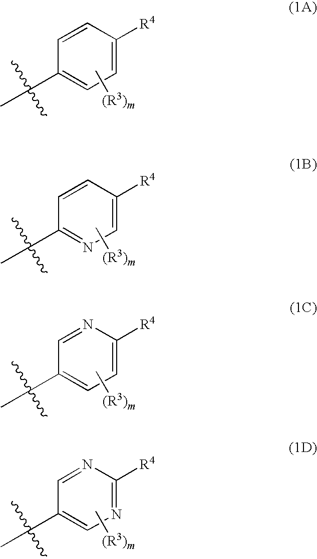
where R³ is (C₁-C₄)alkyl, (C₁-C₄)alkoxy, halo-substituted (C₁-C₄)alkyl, halo-substituted (C₁-C₄)alkoxy, halogen, or hydroxyl, or R³ is taken together with R⁴ to form a 5- to 6-membered carbocyclic fused ring or a 5- to 6-membered heterocyclic fused ring containing 1 to 2 heteroatoms selected from O, S or N;
m is 0 or 1;
R⁴ is hydrogen, halogen, or a chemical moiety selected from the group consisting of:
(ii) taken together with R³ to form a 5- to 6-membered carbocyclic fused ring, a 5- to 6-membered heterocyclic fused ring containing 1 to 2 heteroatoms each independently selected from O, N or S, or a 5- to 6-membered heteroaryl fused ring containing 1 to 2 heteroatoms each independently selected form O, N or S;
(ii) (C₁-C₆)alkyl optionally substituted with hydroxy, cyano, (C₁-C₄)alkoxy, halo-substituted (C₁-C₄)alkyl, halo-substituted (C₁-C₄)alkoxy, or a 3- to 6-membered cycloalkyl group;
(iii) (C₁-C₆)alkoxy optionally substituted with a 3- to 6-membered cycloalkyl group;
(iv) —S(C₁-C₄)alkyl, —SO(C₁-C₄)alkyl, or —SO₂(C₁-C₄)alkyl;
(v) halo-substituted (C₁-C₄)alkyl;
(vi) halo-substituted (C₁-C₄)alkoxy;
(vii) 3- to 5-membered carbocyclic ring optionally substituted with (CH₂)ₙC(O)—O(R⁵), —(CH₂)ₙOH, (C₁-C₄)alkoxy, cyano, or 1 to 2 halogens, where n is 0 or 1, and R⁵ is H or (C₁-C₄)alkyl,
(viii) —C(CH₃)₂—R⁶, where R⁶ is hydroxy, cyano, (C₁-C₆)alkoxy, halo-substituted (C₁-C₆)alkoxy, halogen, —NH₂, NH, oxo —S(C₁-C₄)alkyl, —SO(C₁-C₄)alkyl, —SO₂(C₁-C₄)alkyl, —O—SO₂(C₁-C₄)alkyl, —(CH₂)ₚC(O)—N(R^{6a})(R^{6b}), —(CH₂)ₚNH—C(O)(C₁-C₄)alkyl), —(CH₂)ₚNH—C(O)(C₁-C₄)alkoxy, —(CH₂)ₚC(O)—O(R^{6c}), —(CH₂)ₚOH, a 5- to 6-membered heteroaryl containing 1 to 4 heteroatoms each independently selected from oxygen, nitrogen or sulfur and optionally substituted with (C₁-C₄)alkyl, —(CH₂)ₚC(O)—N(R^{6a})(R^{6b}), —(CH₂)ₚNH—C(O)(C₁-C₄)alkyl), —(CH₂)ₚC(O)—O(R^{6c}), or —(CH₂)ₚOH, where R^{6a}, R^{6b}, and R^{6c} are each independently selected from hydrogen or (C₁-C₄)alkyl and p is 0, 1, or 2;
(ix) —(CH₂)_{q}—C(OH)(R⁷)(R⁸), where q is 0 or 1 and R⁷ and R⁸ are each independently hydrogen, (C₁-C₄)alkyl, or a halo-substituted (C₁-C₄)alkyl;
(x) —(CH₂)ᵣ—C(O)—R⁹, where R⁹ is —NR^{9a}R^{9b} or —OR^{9b}, where r is 0 or 1, R^{9a}, R^{9b} and R^{9c} are each independently selected form hydrogen or (C₁-C₄)alkyl; and
(xi) a group of formula (1E) 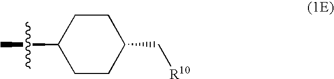
wherein R¹⁰ is
(a) cyano;
(b) —C(O)—N(R⁵)(R⁶);
(c) —C(O)O(R⁵);
(d) —(CH₂)ₙOH where n is 0, 1, or 2;
(e) a 5- to 6-membered heteroaryl containing 1 to 3 heteroatoms each independently selected from oxygen, nitrogen or sulfur, wherein said heteroaryl is optionally substituted with 1 to 3 substituents each independently selected from OH, halogen, or (C₁-C₄)alkyl;
or a pharmaceutically acceptable salt thereof.

3. The compound of claim 1 or 2 wherein:
R¹ is hydrogen, methoxy, or methyl; R² is hydrogen or methyl; R³ is halogen or (C₁-C₄)alkyl; and m is 0 or 1;
or a pharmaceutically acceptable salt thereof.

4. The compound of claim 3 wherein A is a group of formula (1A), 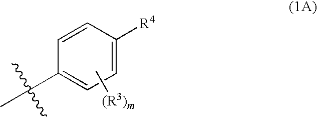
where R⁴ is (C₁-C₆)alkyl optionally substituted with one or more substitutents selected from the group consisting of hydroxy, cyano, (C₁-C₆)alkoxy, halo-substituted (C₁-C₆)alkoxy, halogen, —NH₂, NH, oxo —S(C₁-C₄)alkyl, —SO(C₁-C₄)alkyl, —SO₂(C₁-C₄)alkyl, —O—SO₂(C₁-C₄)alkyl, —(CH₂)ₚC(O)—N(R^{6a})(R^{6b}), —(CH₂)ₚNH—C(O)(C₁-C₄)alkyl), —(CH₂)ₚNH—C(O)(C₁-C₄)alkoxy, —(CH₂)ₚC(O)—O(R^{6c}), —(CH₂)ₚOH, a 3- to 6-membered carbocyclic group, an aryl group, and a 5- to 6-membered heteroaryl group containing 1 to 4 heteroatoms each independently selected from O, S, and N, wherein the carbocyclic, aryl and heteroaryl groups are optionally substituted with (C₁-C₄)alkyl, —(CH₂)ₚC(O)—N(R^{6a})(R^{6b}), —(CH₂)ₚNH—C(O)(C₁-C₄)alkyl), —(CH₂)ₚC(O)—O(R^{6c}), —(CH₂)ₚNH—C(O)(C₁-C₄)alkoxy, or —(CH₂)ₚOH;
or a pharmaceutically acceptable salt thereof.

5. The compound of claim 4 wherein R⁴ is (C₁-C₆)alkyl optionally substituted with one or more substitutents selected from the group consisting of hydroxy, cyano, (C₁-C₆)alkoxy, halo-substituted (C₁-C₆)alkoxy, halogen, —NH₂, NH, oxo, —(CH₂)ₚC(O)—N(R^{6a})(R^{6b}), and —(CH₂)ₚC(O)—O(R^{6c});
or a pharmaceutically acceptable salt thereof.

6. The compound of claim 3 wherein A is a group of formula (1A), 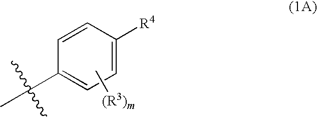
where R⁴ is a 3- to 6-membered carbocyclic ring or a 3- to 6-membered heterocyclic ring containing 1 to 2 heteroatoms each independently selected from O, N or S, wherein the carbocyclic and heterocyclic rings are optionally substituted with one to four substituents selected from the group consisting of —(CH₂)ₙC(O)—O(R⁵), —(CH₂)ₙOH, (C₁-C₄)alkoxy, —(CH₂)ₙC(O)—N(R⁵)(R⁶), —(CH₂)ₙOH, (C₁-C₄)alkyl, (C₁-C₄)haloalkyl, (C₁-C₄)haloalkoxy, hydroxyl, halogen, cyano, oxo, a 5- to 6-membered heteroaryl containing 1 to 3 heteroatoms each independently selected from O, N and S, and —N(R⁵)(R⁶), wherein said heteroaryl is optionally substituted with 1 to 3 substituents each independently selected from OH, halogen, or (C₁-C₄)alkyl;
or a pharmaceutically acceptable salt thereof.

7. The compound of claim 6 wherein R⁴ is a 3- to 6-membered carbocyclic ring optionally substituted with one or two substituents selected from the group consisting of —(CH₂)ₙC(O)—O(R⁵), —(CH₂)ₙOH, (C₁-C₄)alkoxy, —(CH₂)ₙC(O)—N(R⁵)(R⁶), (C₁-C₄)alkyl, (C₁-C₄)haloalkyl, (C₁-C₄)haloalkoxy, hydroxyl, halogen, cyano, and oxo;
or a pharmaceutically acceptable salt thereof.

8. A compound selected from the group consisting of:
4-amino-6-[4-(cyclopropylmethyl)phenyl]-2-methoxy-7,8-dihydropyrido[4,3-d]pyrimidin-5(6H)-one;
2-[4-(4-amino-2-methoxy-5-oxo-7,8-dihydropyrido[4,3-d]pyrimidin-6(5H)-yl)phenyl]-2-methylpropanoic acid;
4-amino-6-(3,4-dichlorophenyl)-2-methoxy-7,8-dihydropyrido[4,3-d]pyrimidin-5(6H)-one;
2-[4-(4-amino-2-methoxy-5-oxo-7,8-dihydropyrido[4,3-d]pyrimidin-6(5H)-yl)phenyl]-2-methylpropanamide;
2-[4-(4-amino-2-methoxy-5-oxo-7,8-dihydropyrido[4,3-d]pyrimidin-6(5H)-yl)phenyl]-2-methylpropanenitrile;
4-amino-6-[4-(2-hydroxy-1,1-dimethylethyl)phenyl]-2-methoxy-7,8-dihydropyrido[4,3-d]pyrimidin-5(6H)-one;
4-amino-6-[4-(1-hydroxy-1-methylethyl)phenyl]-2-methoxy-7,8-dihydropyrido[4,3-d]pyrimidin-5(6H)-one; and
4-amino-2-methoxy-6-{4-[2,2,2-trifluoro-1-hydroxy-1-(trifluoromethyl)ethyl]-phenyl}-7,8-dihydropyrido[4,3-d]pyrimidin-5(6H)-one;
or a pharmaceutically acceptable salt thereof.

9. A compound selected from the group consisting of:
1-[4-(4-amino-2-methoxy-5-oxo-7,8-dihydropyrido[4,3-d]pyrimidin-6(5H)-yl)phenyl]cyclobutanecarboxylic acid;
4-amino-6-[4-(1-ethyl-1-hydroxypropyl)phenyl]-2-methoxy-7,8-dihydropyrido[4,3-d]pyrimidin-5(6H)-one;
1-[4-(4-amino-2-methoxy-5-oxo-7,8-dihydropyrido[4,3-d]pyrimidin-6(5H)-yl)phenyl]cyclobutanecarbonitrile;
1-[4-(4-amino-2-methoxy-5-oxo-7,8-dihydropyrido[4,3-d]pyrimidin-6(5H)-yl)phenyl]cyclobutanecarboxamide; and
1-[4-(4-amino-2-methoxy-5-oxo-7,8-dihydropyrido[4,3-d]pyrimidin-6(5H)-yl)phenyl]cyclopentanecarboxamide;
or a pharmaceutically acceptable salt thereof.

10. A pharmaceutical composition comprising (i) a compound of any one of the preceding claims; and (ii) a pharmaceutically acceptable excipient, diluent, or carrier.

11. The composition of claim 10 wherein said compound or said therapeutically acceptable salt thereof is present in a pharmaceutically effective amount.

12. The composition of claim 11 further comprising at least one additional pharmaceutical agent selected from the group consisting of an anti-obesity agent and an anti-diabetic agent.

13. The composition of claim 12 wherein said anti-obesity agent is selected from the group consisting of dirlotapide, mitratapide, implitapide, R56918 (CAS No. 403987), CAS No. 913541-47-6, lorcaserin, cetilistat, PYY₃₋₃₆, naltrexone, oleoyl-estrone, obinepitide, pramlintide, tesofensine, leptin, liraglutide, bromocriptine, orlistat, exenatide, AOD-9604 (CAS No. 221231-10-3) and sibutramine; and
said anti-diabetic agent is selected from the group consisting of metformin, acetohexamide, chlorpropamide, diabinese, glibenclamide, glipizide, glyburide, glimepiride, gliclazide, glipentide, gliquidone, glisolamide, tolazamide, tolbutamide, tendamistat, trestatin, acarbose, adiposine, camiglibose, emiglitate, miglitol, voglibose, pradimicin-Q, salbostatin, balaglitazone, ciglitazone, darglitazone, englitazone, isaglitazone, pioglitazone, rosiglitazone, troglitazone, exendin-3, exendin-4, trodusquemine, reservatrol, hyrtiosal extract, sitagliptin, vildagliptin, alogliptin and saxagliptin.

14. A method for treating or delaying the progression or onset of Type 2 diabetes and diabetes-related disorders in animals comprising the step of administering to an animal in need of such treatment a therapeutically effective amount of a compound of any one of claims 1 through 9.

15. A method for treating or delaying the progression or onset of Type 2 diabetes and diabetes-related disorders in animals comprising the step of administering to an animal in need of such treatment a pharmaceutical composition of claim 10.

16. A method for treating a disease, condition or disorder modulated by the inhibition of DGAT-1 in animals comprising the step of administering to an animal in need of such treatment two separate pharmaceutical compositions comprising
(i) a first composition comprising a compound of claim 1 through 9, and a pharmaceutically acceptable excipient, diluent, or carrier; and
(ii) a second composition comprising at least one additional pharmaceutical agent selected from the group consisting of an anti-obesity agent and an anti-diabetic agent, and a pharmaceutically acceptable excipient, diluent, or carrier;
wherein said disease, condition or disorder modulated by the inhibition of DGAT-1 is selected from the group consisting of obesity, obesity-related disorders, Type 2 diabetes, and diabetes-related disorders.

17. The method of claim 16 wherein said anti-obesity agent is selected from the group consisting of dirlotapide, mitratapide, implitapide, R56918 (CAS No. 403987), CAS No. 913541-47-6, lorcaserin, cetilistat, PYY₃₋₃₆, naltrexone, oleoyl-estrone, obinepitide, pramlintide, tesofensine, leptin, liraglutide, bromocriptine, orlistat, exenatide, AOD-9604 (CAS No. 221231-10-3) and sibutramine; and
said anti-diabetic agent is selected form the group consisting of metformin, acetohexamide, chlorpropamide, diabinese, glibenclamide, glipizide, glyburide, glimepiride, gliclazide, glipentide, gliquidone, glisolamide, tolazamide, tolbutamide, tendamistat, trestatin, acarbose, adiposine, camiglibose, emiglitate, miglitol, voglibose, pradimicin-Q, salbostatin, balaglitazone, ciglitazone, darglitazone, englitazone, isaglitazone, pioglitazone, rosiglitazone, troglitazone, exendin-3, exendin-4, trodusquemine, reservatrol, hyrtiosal extract, sitagliptin, vildagliptin, alogliptin and saxagliptin.

18. The method of claim 16 or 17 wherein said first composition and said second composition are administered simultaneously.

19. The method of claim 16 or 17 wherein said first composition and said second composition are administered sequentially and in any order.

20. The use of a compound or a pharmaceutically acceptable salt thereof of claim 1 through 9 in the manufacture of a medicament for treating a disease, condition or disorder that is modulated by the inhibition of DGAT-1.
